SEQUENCE LISTING (95 sequences)

SEQ ID NO: 1 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 2 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 3 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-hydroxy nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 4 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 5 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 6 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-hydroxy nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 7 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-methyl locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 8 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-methyl locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 9 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-methyl locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-methyl nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 10 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-methyl locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-methyl nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide with at least one mesyl phosphoroamidate internucleoside linkage
  modified_base 11: mod_base = OTHER; note = 5-methyl nucleotide with at least one mesyl phosphoroamidate internucleoside linkage
  modified_base 12: mod_base = OTHER; note = Nucleotide with at least one mesyl phosphoroamidate internucleoside linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 11 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-hydroxy nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide with at least one mesyl phosphoroamidate internucleoside linkage
  modified_base 11: mod_base = OTHER; note = 5-methyl nucleotide with at least one mesyl phosphoroamidate internucleoside linkage
  modified_base 12: mod_base = OTHER; note = Nucleotide with at least one mesyl phosphoroamidate internucleoside linkage
  modified_base 13: mod_base = OTHER; note = 5-methyl nucleotide with at least one mesyl phosphoroamidate internucleoside linkage
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 12 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with at least one mesyl phosphoroamidate internucleoside linkage
  modified_base 8: mod_base = OTHER; note = 5-hydroxy nucleotide phosphorothioate
  modified_base 9: mod_base = OTHER; note = Nucleotide with at least one mesyl phosphoroamidate internucleoside linkage
  modified_base 10: mod_base = OTHER; note = 5-methyl nucleotide phosphorothioate
  modified_base 11: mod_base = OTHER; note = 5-methyl nucleotide with at least one mesyl phosphoroamidate internucleoside linkage
  modified_base 12: mod_base = OTHER; note = Nucleotide with phosphorothioate linkage
  modified_base 13: mod_base = OTHER; note = 5-methyl nucleotide with at least one mesyl phosphoroamidate internucleoside linkage
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 13 - DNA, 17 bp, synthetic construct
  source 1..17: mol_type = other DNA
  modified_base 1: mod_base = OTHER; note = Locked nucleotide with at least one mesyl phosphoroamidate internucleoside linkage
  modified_base 2: mod_base = OTHER; note = Locked nucleotide with at least one mesyl phosphoroamidate internucleoside linkage
  modified_base 3: mod_base = OTHER; note = Locked nucleotide with at least one mesyl phosphoroamidate internucleoside linkage
  modified_base 4: mod_base = OTHER; note = Locked nucleotide with at least one mesyl phosphoroamidate internucleoside linkage
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide with at least one mesyl phosphoroamidate internucleoside linkage
  modified_base 15: mod_base = OTHER; note = Locked nucleotide with at least one mesyl phosphoroamidate internucleoside linkage
  modified_base 16: mod_base = OTHER; note = Locked nucleotide with at least one mesyl phosphoroamidate internucleoside linkage
  modified_base 17: mod_base = OTHER; note = Cyclopropyl-5-methyl nucleotide
gataaaacgccgcagac

SEQ ID NO: 14 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with at least one mesyl phosphoroamidate internucleoside linkage
  modified_base 8: mod_base = OTHER; note = 5-hydroxy nucleotide with at least one mesyl phosphoroamidate internucleoside linkage
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 15 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide with at least one mesyl phosphoroamidate internucleoside linkage
  modified_base 11: mod_base = OTHER; note = 5-methyl nucleotide with at least one mesyl phosphoroamidate internucleoside linkage
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 16 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide with at least one mesyl phosphoroamidate internucleoside linkage
  modified_base 12: mod_base = OTHER; note = Nucleotide with at least one mesyl phosphoroamidate internucleoside linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 17 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with at least one mesyl phosphoroamidate internucleoside linkage
  modified_base 13: mod_base = OTHER; note = 5-methyl nucleotide with at least one mesyl phosphoroamidate internucleoside linkage
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 18 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = 2'-O,4'-C-spirocyclopropylene-bridged nucleotide with at least one mesyl phosphoroamidate internucleoside linkage
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Amino bridged nucleotide phosphorothioate
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 19 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = 2'-O,4'-C-spirocyclopropylene-bridged nucleotide with at least one mesyl phosphoroamidate internucleoside linkage
  modified_base 4: mod_base = OTHER; note = Nucleotide with at least one mesyl phosphoroamidate internucleoside linkage
  modified_base 5: mod_base = OTHER; note = 5-methyl nucleotide phosphorothioate
  modified_base 6: mod_base = OTHER; note = Amino bridged nucleotide phosphorothioate
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 20 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = 2'-O,4'-C-spirocyclopropylene-bridged nucleotide with at least one mesyl phosphoroamidate internucleoside linkage
  modified_base 4: mod_base = OTHER; note = Nucleotide with at least one mesyl phosphoroamidate internucleoside linkage
  modified_base 5: mod_base = OTHER; note = 5-methyl nucleotide with at least one mesyl phosphoroamidate internucleoside linkage
  modified_base 6: mod_base = OTHER; note = Amino bridged nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 21 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = 2'-O,4'-C-spirocyclopropylene-bridged nucleotide with at least one mesyl phosphoroamidate internucleoside linkage
  modified_base 4: mod_base = OTHER; note = Nucleotide with at least one mesyl phosphoroamidate internucleoside linkage
  modified_base 5: mod_base = OTHER; note = 5-methyl nucleotide with at least one mesyl phosphoroamidate internucleoside linkage
  modified_base 6: mod_base = OTHER; note = Amino bridged nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = 5-methyl nucleotide with at least one mesyl phosphoroamidate internucleoside linkage
  modified_base 9: mod_base = OTHER; note = Nucleotide with at least one mesyl phosphoroamidate internucleoside linkage
  modified_base 10: mod_base = OTHER; note = 5-methyl nucleotide with at least one mesyl phosphoroamidate internucleoside linkage
  modified_base 11: mod_base = OTHER; note = 5-methyl nucleotide with at least one mesyl phosphoroamidate internucleoside linkage
  modified_base 12: mod_base = OTHER; note = Nucleotide with at least one mesyl phosphoroamidate internucleoside linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 22 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = 2'-O,4'-C-spirocyclopropylene-bridged nucleotide with at least one mesyl phosphoroamidate internucleoside linkage
  modified_base 4: mod_base = OTHER; note = Nucleotide with phosphorothioate linkage
  modified_base 5: mod_base = OTHER; note = 5-methyl nucleotide with at least one mesyl phosphoroamidate internucleoside linkage
  modified_base 6: mod_base = OTHER; note = Amino bridged nucleotide phosphorothioate
  modified_base 7: mod_base = OTHER; note = Nucleotide with at least one mesyl phosphoroamidate internucleoside linkage
  modified_base 8: mod_base = OTHER; note = 5-methyl nucleotide phosphorothioate
  modified_base 9: mod_base = OTHER; note = Nucleotide with at least one mesyl phosphoroamidate internucleoside linkage
  modified_base 10: mod_base = OTHER; note = 5-methyl nucleotide phosphorothioate
  modified_base 11: mod_base = OTHER; note = 5-methyl nucleotide with at least one mesyl phosphoroamidate internucleoside linkage
  modified_base 12: mod_base = OTHER; note = Nucleotide with phosphorothioate linkage
  modified_base 13: mod_base = OTHER; note = Nucleotide with at least one mesyl phosphoroamidate internucleoside linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 23 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = Nucleotide with at least one mesyl phosphoroamidate internucleoside linkage
  modified_base 9: mod_base = OTHER; note = 5-hydroxy nucleotide with at least one mesyl phosphoroamidate internucleoside linkage
  modified_base 10: mod_base = OTHER; note = Nucleotide with at least one mesyl phosphoroamidate internucleoside linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Cyclopropyl-5-methyl nucleotide
agataaaacgccgcagac

SEQ ID NO: 24 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide with at least one mesyl phosphoroamidate internucleoside linkage
  modified_base 7: mod_base = OTHER; note = Nucleotide with at least one mesyl phosphoroamidate internucleoside linkage
  modified_base 8: mod_base = OTHER; note = Nucleotide with at least one mesyl phosphoroamidate internucleoside linkage
  modified_base 9: mod_base = OTHER; note = 5-hydroxy nucleotide with at least one mesyl phosphoroamidate internucleoside linkage
  modified_base 10: mod_base = OTHER; note = Nucleotide with at least one mesyl phosphoroamidate internucleoside linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 25 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = G-clamp nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 26 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = G-clamp nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 27 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = G-clamp nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 28 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = G-clamp nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 29 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = G-clamp nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 30 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = G-clamp nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = G-clamp nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 31 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = G-clamp nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 32 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = G-clamp nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 33 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = G-clamp nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 34 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = G-clamp nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 35 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = G-clamp nucleotide
gataaaacgccgcagac

SEQ ID NO: 36 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = G-clamp nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = G-clamp nucleotide
gataaaacgccgcagac

SEQ ID NO: 37 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methylpropyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 38 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methylpropyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 39 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methylpropyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 40 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methylpropyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 41 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methylpropyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 42 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methylpropyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 43 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methylpropyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 44 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methylpropyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 45 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = N-t-butyl nucleotide
  modified_base 3: mod_base = OTHER; note = Nucleotide with phosphorothioate linkage
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
ggataaaacgccgcagac

SEQ ID NO: 46 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
gagtaaaacgccgcagac

SEQ ID NO: 47 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-t-butyl nucleotide
  modified_base 5: mod_base = OTHER; note = Nucleotide with phosphorothioate linkage
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
gatgaaaacgccgcagac

SEQ ID NO: 48 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = N-t-butyl nucleotide
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
gatagaaacgccgcagac

SEQ ID NO: 49 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = N-t-butyl nucleotide
  modified_base 15: mod_base = OTHER; note = Nucleotide with phosphorothioate linkage
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
gataaaacgccgcgagac

SEQ ID NO: 50 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = N-t-butyl nucleotide
  modified_base 17: mod_base = OTHER; note = Nucleotide with phosphorothioate linkage
  modified_base 18: mod_base = OTHER; note = 5-methyl locked nucleotide
gataaaacgccgcaggac

SEQ ID NO: 51 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = N-t-butyl nucleotide
  modified_base 18: mod_base = OTHER; note = 5-methyl nucleotide
gataaaacgccgcagagc

SEQ ID NO: 52 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = N-t-butyl nucleotide
  modified_base 3: mod_base = OTHER; note = Nucleotide with phosphorothioate linkage
  modified_base 4: mod_base = OTHER; note = Locked nucleotide phosphorothioate
  modified_base 5: mod_base = OTHER; note = Nucleotide with phosphorothioate linkage
  modified_base 6: mod_base = OTHER; note = 5-methyl nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide
ggattcagcgccgacggg

SEQ ID NO: 53 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = 5-methyl nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = 5-methyl locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide
gagttcagcgccgacggg

SEQ ID NO: 54 - DNA, 18 bp, synthetic construct
  source 1..18: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = N-t-butyl nucleotide
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide
gattcagcgccgagcggg

SEQ ID NO: 55 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = 5-methyl locked nucleotide
aggataaaacgccgcagac

SEQ ID NO: 56 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-hydroxy nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = 5-methyl locked nucleotide
aggataaaacgccgcagac

SEQ ID NO: 57 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = Cyclopropyl-5-methyl nucleotide
aggataaaacgccgcagac

SEQ ID NO: 58 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = N-t-butyl nucleotide
  modified_base 4: mod_base = OTHER; note = Nucleotide with phosphorothioate linkage
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-hydroxy nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = Cyclopropyl-5-methyl nucleotide
aggataaaacgccgcagac

SEQ ID NO: 59 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 60 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-t-butyl nucleotide
  modified_base 5: mod_base = OTHER; note = Nucleotide with phosphorothioate linkage
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = 5-methyl locked nucleotide
agagtaaaacgccgcagac

SEQ ID NO: 61 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-t-butyl nucleotide
  modified_base 5: mod_base = OTHER; note = Nucleotide with phosphorothioate linkage
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-hydroxy nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = 5-methyl locked nucleotide
agagtaaaacgccgcagac

SEQ ID NO: 62 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-t-butyl nucleotide
  modified_base 5: mod_base = OTHER; note = Nucleotide with phosphorothioate linkage
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = Cyclopropyl-5-methyl nucleotide
agagtaaaacgccgcagac

SEQ ID NO: 63 - DNA, 19 bp, synthetic construct
  source 1..19: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-t-butyl nucleotide
  modified_base 5: mod_base = OTHER; note = Nucleotide with phosphorothioate linkage
  modified_base 6: mod_base = OTHER; note = Locked nucleotide phosphorothioate
  modified_base 7: mod_base = OTHER; note = Locked nucleotide phosphorothioate
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = 5-hydroxy nucleotide phosphorothioate
  modified_base 11: mod_base = OTHER; note = Nucleotide with phosphorothioate linkage
  modified_base 12: mod_base = OTHER; note = 5-methyl nucleotide phosphorothioate
  modified_base 13: mod_base = OTHER; note = 5-methyl nucleotide phosphorothioate
  modified_base 14: mod_base = OTHER; note = Nucleotide with phosphorothioate linkage
  modified_base 15: mod_base = OTHER; note = 5-methyl nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Locked nucleotide phosphorothioate
  modified_base 19: mod_base = OTHER; note = Cyclopropyl-5-methyl nucleotide
agagtaaaacgccgcagac

SEQ ID NO: 64 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Cyclopropyl-5-methyl nucleotide
gataaaacgccgcagac

SEQ ID NO: 65 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 66 - DNA, 18 bp, synthetic construct
  source 1..18: mol_type = other DNA
  modified_base 1: mod_base = OTHER; note = N-methyl locked nucleotide
  modified_base 2: mod_base = OTHER; note = Nucleotide with phosphorothioate linkage
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 67 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = N-methyl locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 68 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = N-methyl locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 69 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = N-methyl locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 70 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = N-methyl locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 71 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = N-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 72 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = N-methyl locked nucleotide phosphorothioate
  modified_base 16: mod_base = OTHER; note = Locked nucleotide phosphorothioate
  modified_base 17: mod_base = OTHER; note = 5-methyl locked nucleotide
gataaaacgccgcagac

SEQ ID NO: 73 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = N-methyl locked nucleotide phosphorothioate
  modified_base 17: mod_base = OTHER; note = 5-methyl locked nucleotide
gataaaacgccgcagac

SEQ ID NO: 74 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide phosphorothioate
  modified_base 14: mod_base = OTHER; note = Locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = N-methyl-5-methyl locked nucleotide
gataaaacgccgcagac

SEQ ID NO: 75 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Cyclopropyl nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = 8-amino nucleotide phosphorothioate
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 76 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 77 - DNA, 17 bp, synthetic construct
  source 1..17: mol_type = other DNA
  modified_base 1: mod_base = OTHER; note = N-methyl locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 78 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = N-methyl locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 79 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = N-methyl locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 80 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = N-methyl locked nucleotide
gattcagcgccgacggg

SEQ ID NO: 81 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = Locked nucleotide phosphorothioate
  modified_base 16: mod_base = OTHER; note = N-methyl locked nucleotide phosphorothioate
  modified_base 17: mod_base = OTHER; note = Locked nucleotide
gattcagcgccgacggg

SEQ ID NO: 82 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = 5-methyl locked nucleotide phosphorothioate
  modified_base 15: mod_base = OTHER; note = N-methyl locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 83 - DNA, 17 bp, synthetic construct
  source 1..17: mol_type = other DNA
  modified_base 1: mod_base = OTHER; note = Locked nucleotide phosphorothioate
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Nucleotide with phosphorothioate linkage
  modified_base 5: mod_base = OTHER; note = 5-methyl nucleotide phosphorothioate
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-methyl nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = N-methyl-5-methyl locked nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide
gattcagcgccgacggg

SEQ ID NO: 84 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = N-methyl locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 85 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = N-methyl locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-hydroxy nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 86 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = Locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-methyl nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = 5-methyl locked nucleotide
agataaaacgccgcagac

SEQ ID NO: 87 - DNA, 18 bp, synthetic construct
  source 1..18: mol_type = other DNA
  modified_base 1: mod_base = OTHER; note = 6-(5-(((2R,3R,4R,5R,6R)-3-acetamido-4,5-dihydroxy-6 -(hydroxymethyl)tetrahydro-2H-pyran-2-yl)oxy)pentanamido)- N-(6-(4-(hydroxymethyl)-4-(methoxymethyl)piperidin-1-yl)-6 -oxohexyl)hexanamide 2'-O-methoxy nucleotide
  modified_base 2: mod_base = OTHER; note = Locked nucleotide phosphorothioate
  modified_base 3: mod_base = OTHER; note = Locked nucleotide phosphorothioate
  modified_base 4: mod_base = OTHER; note = Locked nucleotide phosphorothioate
  modified_base 5: mod_base = OTHER; note = N-methyl locked nucleotide phosphorothioate
  modified_base 6: mod_base = OTHER; note = Locked nucleotide phosphorothioate
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = 5-hydroxy nucleotide phosphorothioate
  modified_base 10: mod_base = OTHER; note = Nucleotide with phosphorothioate linkage
  modified_base 11: mod_base = OTHER; note = 5-methyl nucleotide phosphorothioate
  modified_base 12: mod_base = OTHER; note = 5-methyl nucleotide phosphorothioate
  modified_base 13: mod_base = OTHER; note = Nucleotide with phosphorothioate linkage
  modified_base 14: mod_base = OTHER; note = 5-methyl nucleotide phosphorothioate
  modified_base 15: mod_base = OTHER; note = Locked nucleotide phosphorothioate
  modified_base 16: mod_base = OTHER; note = Locked nucleotide phosphorothioate
  modified_base 17: mod_base = OTHER; note = Locked nucleotide phosphorothioate
  modified_base 18: mod_base = OTHER; note = Cyclopropyl-5-methyl nucleotide
agataaaacgccgcagac

SEQ ID NO: 88 - DNA, 17 bp, synthetic construct
  source 1..17: mol_type = other DNA
  modified_base 1: mod_base = OTHER; note = Locked nucleotide with at least one mesyl phosphoroamidate internucleoside linkage
  modified_base 2: mod_base = OTHER; note = Locked nucleotide with at least one mesyl phosphoroamidate internucleoside linkage
  modified_base 3: mod_base = OTHER; note = Locked nucleotide with at least one mesyl phosphoroamidate internucleoside linkage
  modified_base 4: mod_base = OTHER; note = Locked nucleotide with at least one mesyl phosphoroamidate internucleoside linkage
  modified_base 5: mod_base = OTHER; note = Locked nucleotide with at least one mesyl phosphoroamidate internucleoside linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = 5-hydroxy nucleotide phosphorothioate
  modified_base 9: mod_base = OTHER; note = Nucleotide with phosphorothioate linkage
  modified_base 10: mod_base = OTHER; note = 5-methyl nucleotide phosphorothioate
  modified_base 11: mod_base = OTHER; note = 5-methyl nucleotide phosphorothioate
  modified_base 12: mod_base = OTHER; note = Nucleotide with phosphorothioate linkage
  modified_base 13: mod_base = OTHER; note = 5-methyl nucleotide with at least one mesyl phosphoroamidate internucleoside linkage
  modified_base 14: mod_base = OTHER; note = Locked nucleotide with at least one mesyl phosphoroamidate internucleoside linkage
  modified_base 15: mod_base = OTHER; note = Locked nucleotide with at least one mesyl phosphoroamidate internucleoside linkage
  modified_base 16: mod_base = OTHER; note = Locked nucleotide with at least one mesyl phosphoroamidate internucleoside linkage
  modified_base 17: mod_base = OTHER; note = Cyclopropyl-5-methyl nucleotide
gataaaacgccgcagac

SEQ ID NO: 89 - DNA, 3215 bp, Hepatitis B virus
  source 1..3215: mol_type = genomic DNA
ctccaccactttccaccaaactcttcaagatcccagagtcagggccctgtactttcctgctggtggctcaagttccggaacagtaaaccctgctccgactactgcctctcccatatcgtcaatcttctcgaggactggggaccctgtaccgaatatggagagcaccacatcaggattcctaggacccctgctcgtgttacaggcggggtttttcttgttgacaagaatcctcacaataccacagagtctagactcgtggtggacttctctcaattttctagggggagcacccacgtgtcctggccaaaatttgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactaccaaggtatgttgcccgtttgtcctctacttccaggaacatcaactaccagcaccggaccatgcaaaacctgcacaactactgctcaagggacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttataccgctgttaccaattttcttttatctttgggtatacatttaaaccctcacaaaacaaaaagatggggatattcccttaacttcatgggatatgtaattgggagttggggcactttgcctcaggaacatattgtacaaaaaatcaagcaatgttttaggaaacttcctgtaaacaggcctattgattggaaagtatgtcaacraattgtgggtcttttggggtttgccgcccctttcacgcaatgtggatatcctgctttaatgcctttatatgcatgtatacaagctaagcaggcttttactttctcgccaacttacaaggcctttctgtgtaaacaatatctgaacctttaccccgttgctcggcaacggtcaggtctttgccaagtgtttgctgacgcaacccccactggttggggcttggccataggccatcagcgcatgcgtggaacctttgtggctcctctgccgatccatactgcggaactcctagcagcttgttttgctcgcagccggtctggagcaaaacttatcggcaccgacaactctgttgtcctctctcggaaatacacctcctttccatggctgctaggatgtgctgccaactggatcctgcgcgggacgtcctttgtctacgtcccgtcggcgctgaatcccgcggacgacccatctcggggccgtttgggactctaccgtccccttctgcgtctgccgttccgcccgaccacggggcgcacctctctttacgcggtctccccgtctgtgccttctcatctgccggaccgtgtgcacttcgcttcacctctgcacgtcgcatggagaccaccgtgaacgcccacgggaacctgcccaaggtcttgcataagaggactcttggactttcagcaatgtcaacgaccgaccttgaggcatacttcaaagactgtgtgtttactgagtgggaggagttgggggaggaggttaggttaaaggtctttgtactaggaggctgtaggcataaattggtgtgttcaccagcaccatgcaactttttcacctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacggcactcaggcaagcaattctgtgttggggtgagttaatgaatctagccacctgggtgggaagtaatttggaagatccagcatccagggaattagtagtcagctatgtcaacgttaatatgggcctaaaaatcagacaactattgtggtttcacatttcctgtcttacttttgggagagaaactgttcttgaatatttggtgtcttttggagtgtggattcgcactcctcctgcatatagaccacaaaatgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagacttaaattaattatgcctgctaggttttatcccaatgttactaaatatttgcccttagataaagggatcaaaccgtattatccagagtatgtagttaatcattacttccagacgcgacattatttacacactctttggaaggcggggatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccacacaaggacaactggccggacgccaacaaggtgggagtgggagcattcgggccagggttcacccctcctcatgggggactgttggggtggagccctcaggctcagggcatattcacaacagtgccagcagctcctcctcctgcctccaccaatcggcagtcaggaaggcagcctactcccttctctccacctctaagagacactcatcctcaggccatgcagtggaa

SEQ ID NO: 90 - DNA, 3182 bp, Hepatitis B virus
  source 1..3182: mol_type = genomic DNA
aattccacaacctttcaccaaactctgcaagatcccagagtgagaggcctgtatttccctgctggtggctccagttcaggagcagtaaaccctgttccgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaaccaccagcacgggaccatgccgaacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctgaattttatgggttatgtcattggaagttatgggtccttgccacaagaacacatcatacaaaaaatcaaagaatgttttagaaaacttcctattaacaggcctattgattggaaagtatgtcaacgaattgtgggtcttttgggttttgctgccccatttacacaatgtggttatcctgcgttaatgcccttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctctcccgcaaatatacatcgtatccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccgaatgttgcccaaggtcttacataagaggactcttggactctctgcaatgtcaacgaccgaccttgaggcatacttcaaagactgtttgtttaaagactgggaggagttgggggaggagattagattaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcatctagagacctagtagtcagttatgtcaacactaatatgggcctaaagttcaggcaactcttgtggtttcacatttcttgtctcacttttggaagagaaaccgttatagagtatttggtgtctttcggagtgtggattcgcactcctccagcttatagaccaccaaatgcccctatcctatcaacacttccggaaactactgttgttagacgacgaggcaggtcccctagaagaagaactccctcgcctcgcagacgaaggtctcaatcgccgcgtcgcagaagatctcaatctcgggaacctcaatgttagtattccttggactcataaggtggggaactttactggtctttattcttctactgtacctgtctttaatcctcattggaaaacaccatcttttcctaatatacatttacaccaagacattatcaaaaaatgtgaacagtttgtaggcccacttacagttaatgagaaaagaagattgcaattgattatgcctgctaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacacagcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagacaggaaggcagcctaccccgctgtctccacctttgagaaacactcatcctcaggccatgcagtgg

SEQ ID NO: 91 - DNA, 903 bp, Homo sapiens
  source 1..903: mol_type = genomic DNA
atgaacatcatcctagaaatccttctgcttctgatcaccatcatctactcctacttggagtcgttggtgaagtttttcattcctcagaggagaaaatctgtggctggggagattgttctcattactggagctgggcatggaataggcaggcagactacttatgaatttgcaaaacgacagagcatattggttctgtgggatattaataagcgcggtgtggaggaaactgcagctgagtgccgaaaactaggcgtcactgcgcatgcgtatgtggtagactgcagcaacagagaagagatctatcgctctctaaatcaggtgaagaaagaagtgggtgatgtaacaatcgtggtgaataatgctgggacagtatatccagccgatcttctcagcaccaaggatgaagagattaccaagacatttgaggtcaacatcctaggacatttttggatcacaaaagcacttcttccatcgatgatggagagaaatcatggccacatcgtcacagtggcttcagtgtgcggccacgaagggattccttacctcatcccatattgttccagcaaatttgccgctgttggctttcacagaggtctgacatcagaacttcaggccttgggaaaaactggtatcaaaacctcatgtctctgcccagtttttgtgaatactgggttcaccaaaaatccaagcacaagattatggcctgtattggagacagatgaagtcgtaagaagtctgatagatggaatacttaccaataagaaaatgatttttgttccatcgtatatcaatatctttctgagactacagaagtttcttcctgaacgcgcctcagcgattttaaatcgtatgcagaatattcaatttgaagcagtggttggccacaaaatcaaaatgaaatga

SEQ ID NO: 92 - DNA, 7459 bp, Homo sapiens
  source 1..7459: mol_type = genomic DNA
agtcactgccgcggcgccttgagtctccgggccgccttgccatggctgcccgtggtgtcatcgctccagttggcgagagtttgcgctacgctgagtacttgcagccctcggccaaacggccagacgccgacgtcgaccagcagagactggtaagaagtttgatagctgtaggactgggtgttgcagctcttgcatttgcaggtcgctacgcatttcggatctggaaacctctagaacaagttatcacagaaactgcaaagaagatttcaactcctagcttttcatcctactataaaggaggatttgaacagaaaatgagtaggcgagaagctggtcttattttaggtgtaagcccatctgctggcaaggctaagattagaacagctcataggagagtcatgattttgaatcacccagataaaggtggatctccttacgtagcagccaaaataaatgaagcaaaagacttgctagaaacaaccaccaaacattgatgcttaaggaccacactgaaggaaaaaaaaagaggggacttcgaaaaaaaaaaaagccctgcaaaatattctaaaacatggtcttcttaattttctatatggattgaccacagtcttatcttccaccattaagctgtataacaataaaatgttaatagtcttgctttttattatcttttaaagatctccttaaattctataactgatcttttttcttattttgtttgtgacattcatacatttttaagatttttgttatgttctgaattcccccctacacacacacacacacacacacacacacacacgtgcaaaaaatatgatcaagaatgcaattgggatttgtgagcaatgagtagacctcttattgtttatatttgtaccctcattgtcaatttttttttagggaatttgggactctgcctatataaggtgttttaaatgtcttgagaacaagcactggctgatacctcttggagatatgatctgaaatgtaatggaatttattaaatggtgtttagtaaagtaggggttaaggacttgttaaagaaccccactatctctgagaccctatagccaaagcatgaggacttggagagctactaaaatgattcaggtttacaaaatgagccctgtgaggaaaggttgagagaagtctgaggagtttgtatttaattatagtcttccagtactgtatattcattcattactcattctacaaatatttattgaccccttttgatgtgcaaggcactatcgtgcgtcccctgagagttgcaagtatgaagcagtcatggatcatgaaccaaaggaacttatatgtagaggaaggataaatcacaaatagtgaatactgttagatacagatgatatattttaaaagttcaaaggaagaaaagaatgtgttaaacactgcatgagaggaggaataagtggcatagagctaggctttagaaaagaaaaatattccgataccatatgattggtgaggtaagtgttattctgagatgagaattagcagaaatagatatatcaatcggagtgattagagtgcagggtttctggaaagcaaggtttggacagagtggtcatcaaaggccagccctgtgacttacactgcattaaattaatttcttagaacatagtccctgatcattatcactttactattccaaaggtgagagaacagattcagatagagtgccagcattgtttcccagtattcctttacaaatcttgggttcattccaggtaaactgaactactgcattgtttctatcttaaaatactttttagatatcctagatgcatctttcaacttctaacattctgtagtttaggagttctcaaccttggcattattgacatgttaggccaaataattttttttgtgggaggtctcttgtgcgttttagatgattagcaataatccctgacctgttatctactaaagactagtcgtttctcatcagttgtgacaacaaaaatggttccagatattgccaaatgccctttagaggacagtaatcgcccccagttgagaaccatttcagtaaaactttaattactattttttcttttggtttataaaataatgatcctgaattaaattgatggaaccttgaagtcgataaaatatatttcttgctttaaagtccccatacgtgtcctactaattttctcatgctttagtgttttcacttttctcctgttatccttgtacctaagaatgccatcccaatccccagatgtccacctgcccaaagtctaggcatagctgaaggccaagctaaaatgtatccctctttttctggtacatgcagcaaaagtaatatgaattatcagctttctgagagcaggcattgtatctgtcttgtttggtgttacattggcacccaataaatatttgttgagtgaatgaataaattcccatagcactttattcttcacatggtacataactataggggctatagcttggtaccttgtgaagcaactcttggtgtaacataccttatttctcatactaaaatgcaagaacctttagagcaaggatcttgccattcatctttgtaacctctttactctggagcacttgcatttagcaggcatcataaagttttacgtaccaagaaaatgttgctgttttctgaatactatgcatcaaaaaatgttaccactaatttttaaagctctgctaaggaatattggggcaccctcagatgcaccttttaattgatgtcatattttcctaatccatactttattcatgagaatttgagtcaccccagcattagcttggaatttccttatttcccatttgctttgcaggtgccttggagtcagatctggttttgaatactatcttcctgttatgtgatcttgggcagttacttaattttctagtcaataacccgtatctataaaatagagaaaataatcctacacaccggggcctgttgtggggcggggagaggggggagggatcgcatttggagatatactaatgtaaatgacaagttaattggtgcagcacaccaacatggctcatgtctacatatgtaacaaacctgcacgttgtgcacatgtgccctagaacttaaagtataataaaaagaaattttaaaaaatcctgtcaaataaggttatagtagagaataaggatgtgtaaagcatttagtcacgtaaatgcttaaaaaaatgtaatttttacttctttcactgcctcatttaattagttttatctttaataataccttggattcagggtaaagtttcagttatgtcccagtaatcatttattttaccctcgaatctgcaatttggatagaacatggtggggacagctcgtctctattccttgcagcattaacaggctggaggcaccacttctctggccagcaagttgggcctggttgttggctgagagcctcagttcctttctgcacaggttcctctttacataggcttctcaacagggctactagagcatcgtcaccatagcagctgtcttataacagagagtggtcggtctgagagacaaaaaatggaagctgccaaattgttctgggtctggaaactgtcagggcatcacttgtgccatattcagttggcctaagaattacagagcctgcctcgattcaaagggagaggatagagaggactgaaggaatcagtgctcatctttaatatgcagcaggacaggtttgggattttttttcccccttgagtctgtgaaggcattacttaagaacaaagtcaggcatgtataattgaactacagttacttgaaatataagcccagaaagtttcagataataaatacaactatttttctgctgttacccttgtacctaaagatgccatcctaatccccagatctccacaactatacctacatagtagaaggttaaaatgtatccctctttttctggtgcatccagcaaaagtaatatcatgaattatgagctctctgagagcaaggatcatatcagtcttgtttattgttgcagtgaacaagtacagttgcagatattcaggagtaattatctaaatggcagtaggcttataaaactgaattttcaccagccacaccctccccccaactccttatctgtaaaaagcttatttgagtggttacctgtcttcagtaaagattgcgcttgcatatttgctgtcattgcatattctgcttaattaagctctgttgatattgcagtttctgtgcatacttacatcttagatgcaatctgagggcctaggaaggccttttaaaaataaaacaattccgattgcagagaaagtgtaagtcaaggacagttaattcaaggggaacatagaaagctatttagattttagttgatggtgccagtcttcagcgtaaagtcaaaagtggagggaagtttagtaaggaaaaaatgttgggcttggaatacattgtttagtcttcaaagcactttactttttatgaaatatattttagacattcagcaaatattgaatacttactatatcaggcagtaaagatataaattcattcttaaaatgtgcaacatgttcaaactgaaaaaaatacattcttaaacaggaaactttttccttcatactttttaattaacaagacatataagagttgcattaatgggcgtgcttatgattgatcacccagcagcatcattagaaataatatattttattcatgtgcagaaatcttttggttgtcctggggaaccttgaacacagaaaagagcttttattgataaggtaattgaacacacttgacaattagcttaatatggtttaataccatttgtgggagaagatgaatcagccaggctctttacgtcaagaatatgaagtttctcttgagtcaaccaacttaagatgagctacggagactgcagtgaaaagttaaatatccaagtacaccagccaatttcacacagtggaaccatgctgtcctcgggcaccctgcacctcgcccaacagtcatcaactagatggaggctcctggctgcaaggaggatttgatgggaatgagtaaatgtgtcagcatagtccgtcccttctaatggaaaagcaacccaaagagcaaatcctattaatggctggatcagtatcatctacttgtcaaaaacattccatgaattatgagtcaaaattttatttatggtggcattacacacattaagagatgaggacttctgttagcataatttattagctggaaaagttgagaaggttctctggactcatttttataggtggaacctaagtgatctggataattgcccaccagcaaaattgctgggcatggtggacaaagaaaatgttccttctaatgattttttatgagctgagtagctattgttcccagctgagtgctcttttcctctttttattgttgctgagcaaaagaatttataaaaagctctttcttttgtattaaaaaccctgctcaattgaaatgcaagttcattaagtaatcttcatttctcttcctgccataataaccctttccctctctgttcgattcaacagtatctagcagcactgctccaaattttaagtctgaacagactatattacatagatgtagagaaatactcaatcttcagcattaagagggagcttaatttcacacgggtggaatatgatcactcaggctagatgttggccataaatttcaaattagtatctcaacttagcaggggggatcaacagtggcaaacttcaattatgacaggataaaaatcacatagagatattggttcaatatggacatctaaactataatgctaaaagccaataattagaataagttcattttaagaaaagcattaataatattagctaacgtttagtacctgtgccaaacattctacctatgttaccttgattttcatagccagcctaagaggtactattatgtatccccattttacaggttaagaaacaggctcagaggagtttaggatcttttccaagattacatagccagtaagtggtggcactaggaaccaaattcagactctgaatcgcatgctgtttatattatattgcactcattctaaatatgtgggaatcagaatgaaggggcttgtatgacttttggctcattttttgatgcatgtgacctgggattataaatgtgaaattaggtttacgaaaggatccagtgtcattgtgcatcatgggcaaggagtacctaatctctttaattcttccctggaagcttacgatgtccatccaagtgcacatagcaaaagttctgttgtaaagtttagcagagtgactttctttgactcagagtgatgacggaggaagctttgataagattttatctgaaatgttcatggacaagagctttcaaggagaacatccagagcaaggttctgaagacagctcatgaaggtgaagcagcagacctggcacaagaaatgaagagagagctcagtgtattaaagatgaaaacaagaaaaccgaatatattgaaaggagcagagaggcaatgaaaacaagacaactgaaatgaggtaacttgcagcaattgaaagggaatttcagtacttttatagaattcttaaaaattgtttcctgctgtttattttcaattttgaacagggttatttgtccatgccatactttttttgccaaattccaaaattgtgtatagttctatagttgtctggtggagtcaatggaactttagttaccagtctaagaatgtgtctttgagattgtccagttaattctctatttccagtagctgtaataaatggtgaaaaggtttctgactcctggagaaagtttctaactccttatgactaatattcataacagacttgtgagttccttgaacatggatacacctatatgcaagagtgtattccaaagctaactcagtgatctttccatttatctattcttggattagtggtgcctttgctctttccttctgtaaatgtgaatagttaagagttgactgcagaagtgtttacactttggcttccatgcctctggaatgtttgtgctttggtggtgagatgtgagactatatttgtatagtctgcatctctcaggctgccccagaatgttgtacagtgcagtgctgaagaaagcagcaggtacacacagaaatgcagcctttcctggttaaccctgcttggatctgagttacactttgtttcctgacttcttgggacttaggtaatcagtttgccttctactctatctcattttgtactcgcttacatactacattcttgtttgggctttcgtttcttcttgtaagcagagattttttaaaatccaatatgtgaaaatacggatgcactacaattaaataaataaaatgctgttgtgtttgttttgctttaaaattgtaaaggataaacaataagatagttttatctatgtggttttcccgatgcagttaaaataaaacctaatctgctaaaattgaa

SEQ ID NO: 93 - DNA, 1906 bp, Homo sapiens
  source 1..1906: mol_type = genomic DNA
gctttccggcggttgcaccgggccggggtgccagcgcccgccttcccgtttcctcccgttccgcagcgcgcccacggcctgtgaccccggcgaccgctccccagtgacgagagagcggggccgggcgctgctccggcctgacctgcgaagggacctcggtccagtcccctgttgcgccgcgcccccgtccgtccgtgcgcgggccagtcaggggccagtgtctcgagcggtcgaggtcgcagacctagaggcgccccacaggccggcccggggcgctgggagcgccggccgcgggccgggtggggatgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagcagaatttttccaagcagagaatgaggggaaaggtgttctagacacaggcaggcacatgccaggtgctggaaaaagaagagagaaaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagataggccttgcttgctgccttctggattcttggcccgcacagagctggggctgagggatggactgatgcttttagctcaaacgtggcttttagacagatttgttcatagaccctctcaagtgccctctccgagctggtaggcattccagctcctccgtgcttcctcagttacacaaaggacctcagctgcttctcccacttggccaagcagggaggaagaagcttaggcagggctctctttccttcttgccttcagatgttctctcccaggggctggcttcaggagggagcatagaaggcaggtgagcaaccagttggctaggggagcagggggcccaccagagctgtggagaggggaccctaagactcctcggcctggctcctacccaccgcccttgccgaaccaggagctgctcactacctcctcagggatggccgttggccacgtcttccttctgcctgagcttcccccccaccacaggccctttcctcaggcaaggtctggcctcaggtgggccgcaggcgggaaaagcagcccttggccagaagtcaagcccagccacgtggagcctagagtgagggcctgaggtctggctgcttgcccccatgctggcgccaacaacttctccatcctttctgcctctcaacatcacttgaatcctagggcctgggttttcatgtttttgaaacagaaccataaagcatatgtgttggcttgttgtaaaa

SEQ ID NO: 94 - DNA, 2926 bp, Homo sapiens
  source 1..2926: mol_type = genomic DNA
agaagaaaacagttccacgttgcttgaaattgaaaatcaagataaaaatgttcacaattaagctccttctttttattgttcctctagttatttcctccagaattgatcaagacaattcatcatttgattctctatctccagagccaaaatcaagatttgctatgttagacgatgtaaaaattttagccaatggcctccttcagttgggacatggtcttaaagactttgtccataagacgaagggccaaattaatgacatatttcaaaaactcaacatatttgatcagtctttttatgatctatcgctgcaaaccagtgaaatcaaagaagaagaaaaggaactgagaagaactacatataaactacaagtcaaaaatgaagaggtaaagaatatgtcacttgaactcaactcaaaacttgaaagcctcctagaagaaaaaattctacttcaacaaaaagtgaaatatttagaagagcaactaactaacttaattcaaaatcaacctgaaactccagaacacccagaagtaacttcacttaaaacttttgtagaaaaacaagataatagcatcaaagaccttctccagaccgtggaagaccaatataaacaattaaaccaacagcatagtcaaataaaagaaatagaaaatcagctcagaaggactagtattcaagaacccacagaaatttctctatcttccaagccaagagcaccaagaactactccctttcttcagttgaatgaaataagaaatgtaaaacatgatggcattcctgctgaatgtaccaccatttataacagaggtgaacatacaagtggcatgtatgccatcagacccagcaactctcaagtttttcatgtctactgtgatgttatatcaggtagtccatggacattaattcaacatcgaatagatggatcacaaaacttcaatgaaacgtgggagaactacaaatatggttttgggaggcttgatggagaattttggttgggcctagagaagatatactccatagtgaagcaatctaattatgttttacgaattgagttggaagactggaaagacaacaaacattatattgaatattctttttacttgggaaatcacgaaaccaactatacgctacatctagttgcgattactggcaatgtccccaatgcaatcccggaaaacaaagatttggtgttttctacttgggatcacaaagcaaaaggacacttcaactgtccagagggttattcaggaggctggtggtggcatgatgagtgtggagaaaacaacctaaatggtaaatataacaaaccaagagcaaaatctaagccagagaggagaagaggattatcttggaagtctcaaaatggaaggttatactctataaaatcaaccaaaatgttgatccatccaacagattcagaaagctttgaatgaactgaggcaaatttaaaaggcaataatttaaacattaacctcattccaagttaatgtggtctaataatctggtattaaatccttaagagaaagcttgagaaatagattttttttatcttaaagtcactgtctatttaagattaaacatacaatcacataaccttaaagaataccgtttacatttctcaatcaaaattcttataatactatttgttttaaattttgtgatgtgggaatcaattttagatggtcacaatctagattataatcaataggtgaacttattaaataacttttctaaataaaaaatttagagacttttattttaaaaggcatcatatgagctaatatcacaactttcccagtttaaaaaactagtactcttgttaaaactctaaacttgactaaatacagaggactggtaattgtacagttcttaaatgttgtagtattaatttcaaaactaaaaatcgtcagcacagagtatgtgtaaaaatctgtaatacaaatttttaaactgatgcttcattttgctacaaaataatttggagtaaatgtttgatatgatttatttatgaaacctaatgaagcagaattaaatactgtattaaaataagttcgctgtctttaaacaaatggagatgactactaagtcacattgactttaacatgaggtatcactataccttatttgttaaaatatatactgtatacattttatatattttaacacttaatactatgaaaacaaataattgtaaaggaatcttgtcagattacagtaagaatgaacatatttgtggcatcgagttaaagtttatatttcccctaaatatgctgtgattctaatacattcgtgtaggttttcaagtagaaataaacctcgtaacaagttactgaacgtttaaacagcctgacaagcatgtatatatgtttaaaattcaataaacaaagacccagtccctaaattatagaaatttaaattattcttgcatgtttatcgacatcacaacagatccctaaatccctaaatccctaaagattagatacaaattttttaccacagtatcacttgtcagaatttatttttaaatatgattttttaaaactgccagtaagaaattttaaattaaacccatttgttaaaggatatagtgcccaagttatatggtgacctacctttgtcaatacttagcattatgtatttcaaattatccaatatacatgtcatatatatttttatatgtcacatatataaaagatatgtatgatctatgtgaatcctaagtaaatattttgttccagaaaagtacaaaataataaaggtaaaaataatctataattttcaggaccacagactaagctgtcgaaattaacgctgatttttttagggccagaataccaaaatggctcctctcttcccccaaaattggacaatttcaaatgcaaaataattcattatttaatatatgagttgcttcctctatttggtttcc

SEQ ID NO: 95 - DNA, 2336 bp, Homo sapiens
  source 1..2336: mol_type = genomic DNA
tgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactgagccagccttcggggccaattccctggaggaaccagctgcaaatcacttttttgctctgtaaatttggaagtgtcatgggtgtctgtgggttatttaaaagaaattataacaattttgctaaaccattacaatgttaggtcttttttaagaaggaaaaagtcagtatttcaagttctttcacttccagcttgccctgttctaggtggtggctaaatctgggcctaatctgggtggctcagctaacctctcttcttcccttcctgaagtgacaaaggaaactcagtcttcttggggaagaaggattgccattagtgacttggaccagttagatgattcactttttgcccctagggatgagaggcgaaagccacttctcatacaagcccctttattgccactaccccacgctcgtctagtcctgaaactgcaggaccagtttctctgccaaggggaggagttggagagcacagttgccccgttgtgtgagggcagtagtaggcatctggaatgctccagtttgatctcccttctgccacccctacctcacccctagtcactcatatcggagcctggactggcctccaggatgaggatgggggtggcaatgacaccctgcaggggaaaggactgccccccatgcaccattgcagggaggatgccgccaccatgagctaggtggagtaactggtttttcttgggtggctgatgacatggatgcagcacagactcagccttggcctggagcacatgcttactggtggcctcagtttaccttccccagatcctagattctggatgtgaggaagagatccctcttcagaaggggcctggccttctgagcagcagattagttccaaagcaggtggcccccgaacccaagcctcacttttctgtgccttcctgagggggttgggccggggaggaaacccaaccctctcctgtgtgttctgttatctcttgatgagatcattgcaccatgtcagacttttgtatatgccttgaaaataaatgaaagtgagaatcctctaaaaaaaaaaaa

(19) United States
(12) Patent Application Publication
(10) Pub. No.: US 2023/0383296 A1
(43) Pub. Date: Nov. 30, 2023

(54) MODIFIED GAPMER OLIGOMERS AND METHODS OF USE THEREOF

(71) Applicant: ALIGOS THERAPEUTICS, INC., South San Francisco, CA (US)

(72) Inventors: Leonid Beigelman, San Mateo, CA (US); Vivek Kumar Rajwanshi, Cupertino, CA (US); Laxman Eltepu, San Ramon, CA (US); Saul Martinez Montero, San Bruno, CA (US); Jin Hong, Pacifica, CA (US)

(73) Assignee: ALIGOS THERAPEUTICS, INC., South San Francisco, CA (US)

(21) Appl. No.: 18/122,433

(22) Filed: Mar. 16, 2023

Related U.S. Application Data
(60) Provisional application No. 63/321,019, filed on Mar. 17, 2022.

(51) Int. Cl. C12N 15/113; A61P 31/20

(52) U.S. Cl. CPC C12N 15/1131; A61P 31/20; C12N 15/1137; C12N 15/1136; C12N 2310/11; C12N 2310/315; C12N 2310/314; C12N 2310/3231; C12N 2310/341; C12N 2320/32; C12N 2320/51; C12N 2320/53; C12N 2310/351

(57) ABSTRACT

The disclosure includes antisense oligonucleotides (ASOs), including gapmer ASOs, and methods of making and using the same.

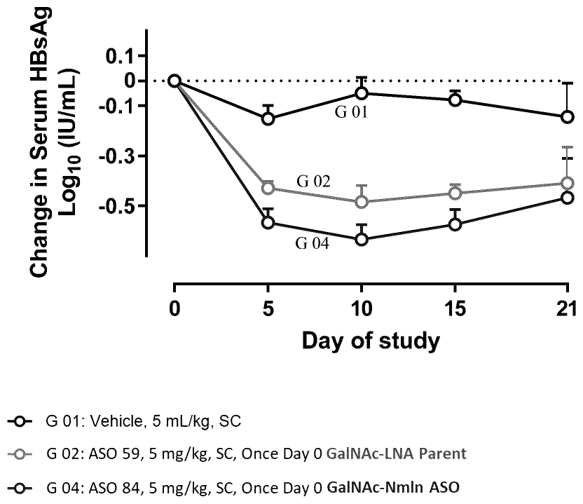

### CROSS-REFERENCE TO RELATED APPLICATION

This application claims priority under 35 U.S.C. § 119 to Provisional Application Ser. No. 63/321,019 filed Mar. 17, 2022, the disclosure of which is incorporated herein by reference.

### SEQUENCE LISTING

The instant application contains a Sequence Listing which has been submitted electronically in XML format and is hereby incorporated by reference in its entirety. Said XML copy, created on Apr. 26, 2023, is named 122400-0355_SL.xml and is 972,753 bytes in size.

### BACKGROUND

The following discussion is provided to aid the reader in understanding the disclosure and is not admitted to describe or constitute prior art thereto.

About 300 million people are chronically infected with HBV worldwide. HBsAg loss, a key aspect of “functional cure” is the goal of many new therapies. Antisense oligonucleotides have been demonstrated to be an effective modality in reducing HBsAg in animal models and clinical studies with these molecules are ongoing.

However, the treatments of HBV with antisense oligonucleotides still suffer from, e.g., nuclease degradation and liver toxicity. Thus, there is a need in the art to discover antisense oligonucleotides having greater resistance to nuclease degradation and improved liver safety profiles.

### SUMMARY

The present disclosure relates to compounds and compositions containing oligonucleotides and their use in preventing or treating diseases and conditions, e.g., hepatitis B (HBV).

In one aspect, the present disclosure provides An antisense oligonucleotide (ASO), comprising 14-22 nucleotide units and:
(a) a central region (B′) comprising 6 or more contiguous DNA nucleotides;
(b) a 5′-wing region (A′) comprising 2 to 6 locked nucleotides or 2′ substituted nucleosides; and
(c) a 3′-wing region (C′) comprising 2 to 6 locked nucleotides or 2′ substituted nucleosides; wherein the central region of the ASO is at least 80% complementary or hybridizes to a target RNA sequence; and wherein the ASO comprises at least one modified nucleotide selected from:

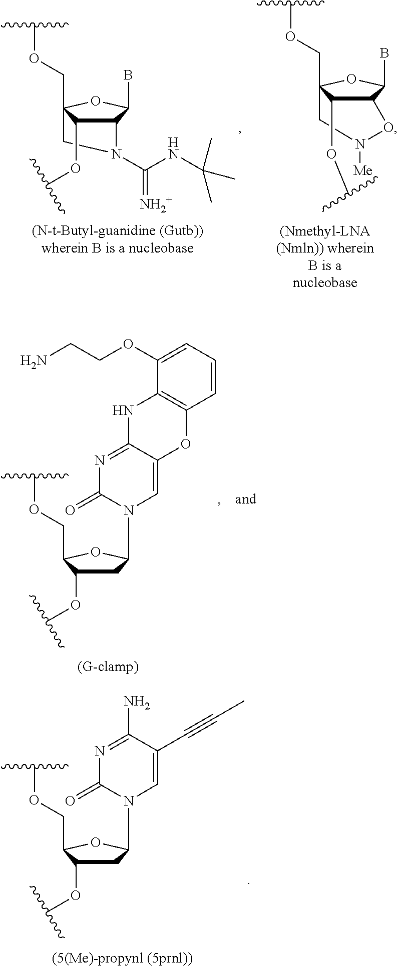

In another aspect, the present disclosure provides antisense oligonucleotides (ASOs), comprising 14-22 nucleotide units and: (a) a central region (B′) comprising 6 or more contiguous DNA nucleotides, wherein at least one of the contiguous DNA nucleotides is a modified nucleotide selected from Gutb, Nmln, G-clamp, and 5prnl;
(b) a 5′-wing region (A′) comprising 2 to 6 locked nucleotides or 2′ substituted nucleosides; and
(c) a 3′-wing region (C′) comprising 2 to 6 locked nucleotides or 2′ substituted nucleosides; wherein the central region of the ASO is at least 80% complementary or hybridizes to a target RNA sequence.

In another aspect, the present disclosure provides antisense oligonucleotides (ASOs), comprising 14-22 nucleotide units and: (a) a central region (B′) comprising 6 or more contiguous DNA nucleotides; (b) a 5′-wing region (A′) comprising 2 to 6 locked nucleotides or 2′ substituted nucleosides; and (c) a 3′-wing region (C′) comprising 2 to 6 locked nucleotides or 2′ substituted nucleosides; wherein the central region of the ASO is at least 80% complementary or hybridizes to a target RNA sequence; and wherein (i) the central region (B′) comprises a modified nucleotide selected from G-clamp and 5prnl, (ii) the 5′-wing region (A′) comprises a modified nucleotide selected from Gutb and Nmln, (iii) the 3′-wing region (C′) comprises a modified nucleotide selected from Gutb and Nmln, or (iv) any combination thereof.

In some embodiments, the central region (B′) comprises 2, 3, 4, 5, or 6 or more modified nucleotides.

In some embodiments, the 5′-wing region (A′), the 3′-wing region (C′), or both comprise a modified nucleotide selected from Gutb, Nmln, G-clamp, and 5prnl.

In some embodiments, the ASO molecule further comprises 1 or more phosphorothioate (ps) internucleoside linkages, mesyl phosphoroamidate (yp) internucleoside linkages, or a combination thereof.

In some embodiments, the ASO molecule further comprises at least 1, 2, 3, 4, 5, 6, 7, 8, 9, 10, 11, 12, 13, 14, 15, 16, 17, 18, 19, 20, 21, or 22 phosphorothioate (ps) internucleoside linkages, mesyl phosphoroamidate (yp) internucleoside linkages, or a combination thereof.

In another aspect, the present disclosure provides, antisense oligonucleotides (ASOs) comprising 14-22 nucleotide units, wherein the ASO comprises:
(a) a central region (B′) comprising 6 or more contiguous DNA nucleotides, wherein at least one of the contiguous DNA nucleotides is a modified nucleotide,
(b) a 5′-wing region (A′) comprising 2 to 6 locked nucleotides or 2′ substituted nucleosides, and
(c) a 3′-wing region (C′) comprising 2 to 6 locked nucleotides or 2′ substituted nucleosides, wherein the central region of the ASO is at least 80% complementary or hybridizes to a target RNA sequence, and wherein the ASO comprises 1, 2, 3, 4, 5, 6, 7, 8, 9, 10, 11, 12, 13, 14, 15, or more mesyl phosphoroamidate (yp) internucleoside linkages.

In some embodiments, the ASO comprises at least 1, at least 2, at least 3, at least 4, or at least 5 or more nucleotide(s) selected from:

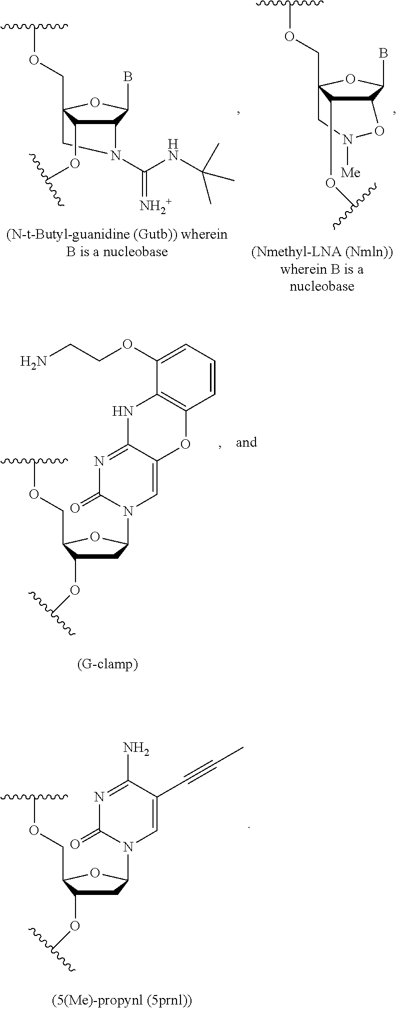

In some embodiments, the ASO molecule further comprises 1 or more phosphorothioate (ps) internucleoside linkages

In some embodiments of any of the foregoing aspects, (i) at least one mesyl phosphoroamidate (yp) internucleotide linkage is between nucleoside positions 3 and 4 from the 5′ end of the ASO molecule; (ii) at least one mesyl phosphoroamidate (yp) internucleotide linkage is between nucleoside positions 5 and 6 from the 5′ end of the ASO molecule; (iii) at least one mesyl phosphoroamidate (yp) internucleotide linkage is between nucleoside positions 6 and 7 from the 5′ end of the ASO molecule; (iv) at least one mesyl phosphoroamidate (yp) internucleotide linkage is between nucleoside positions 7 and 8 from the 5′ end of the ASO molecule; (v) at least one mesyl phosphoroamidate (yp) internucleotide linkage is between nucleoside positions 8 and 9 from the 5′ end of the ASO molecule; (vi) at least one mesyl phosphoroamidate (yp) internucleotide linkage is between nucleoside positions 9 and 10 from the 5′ end of the ASO molecule; or (vii) a combination thereof.

In some embodiments of any of the foregoing aspects, the 5′-wing region (A′), the 3′-wing region (C′), or both comprise at least one mesyl phosphoroamidate (yp) internucleotide linkage.

In some embodiments of any of the foregoing aspects, the ASO molecule further comprises a galactosamine. In some embodiments, the galactosamine is N-acetylgalactosamine (GalNAc) of Formula (VI):

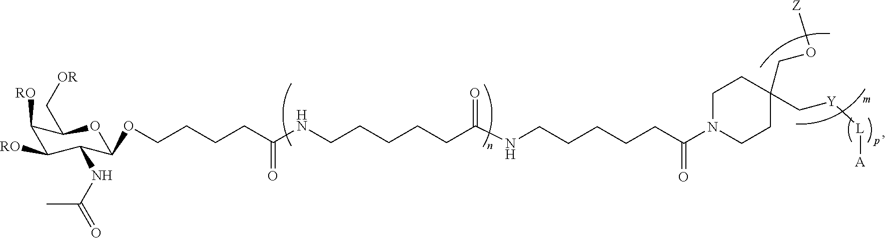

wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide.

In some embodiments, the galactosamine is N-acetylgalactosamine (GalNAc) of Formula (VII):

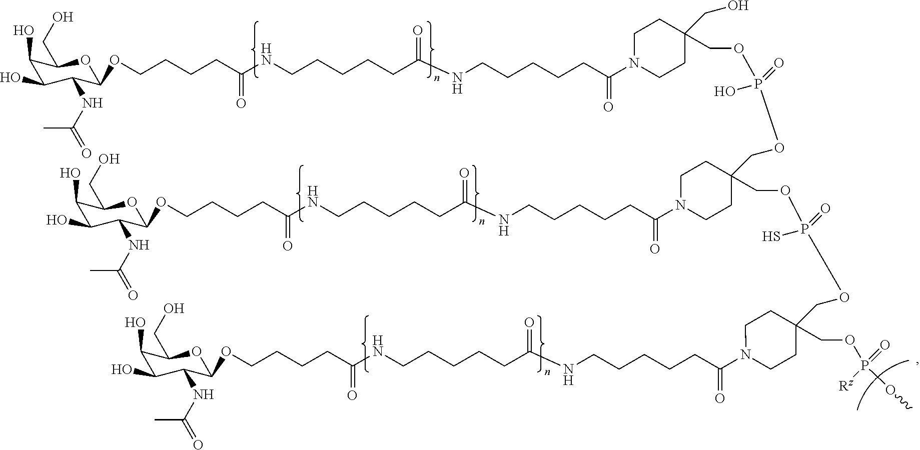

wherein R^{z} is OH or SH; and each n is independently 1 or 2.

In some embodiments of any of the foregoing aspects, (i) the target RNA sequence is a viral gene; (ii) the target RNA sequence is a gene is from a DNA virus; (iii) the target RNA sequence is a gene from a double-stranded DNA (dsDNA) virus; (iv) the target RNA sequence is a gene from a hepadnavirus; (v) the target RNA sequence is a gene from a hepatitis B virus (HBV); (vi) the target RNA sequence is a gene from a HBV of any one of genotypes A-J; or (vii) the target RNA sequence is selected from the S gene or X gene of a HBV.

In some embodiments of any of the foregoing aspects, the target RNA sequence is selected from a gene encoding a Methylation-Controlled J protein (MCJ protein), a gene encoding TAZ, a gene encoding angiopoietin like 3 (ANGPTL3), a gene encoding diacylglycerol acyltransferase 2 (DGAT2), and a gene encoding hydroxysteroid 17-beta dehydrogenase 13 (HSD17B13).

In some embodiments of any of the foregoing aspects, (i) the 5′-wing region of the ASO comprises 2 to 6 phosphorothioate-linked locked nucleosides, (ii) the 3′-wing region of the ASO comprises 2 to 6 phosphorothioate-linked locked nucleosides, or (iii) a combination thereof. In some embodiments, the locked nucleosides are selected from LNA, ScpBNA, AmNA, AmNA (N-Me), GuNA, GuNA (N—R¹¹) where R¹¹ is selected from Me, Et, i-Pr, t-Bu and combinations thereof.

In some embodiments of any of the foregoing aspects, the central region of the ASO comprises at least 5 contiguous phosphorothioate-linked DNA nucleotides, at least 5 contiguous mesyl phosphoroamidate-linked DNA nucleotides, or at least 5 contiguous DNA nucleotides linked by one or more phosphorothioate internucleoside linkages and one or more mesyl phosphoroamidate internucleoside linkages.

In some embodiments of any of the foregoing aspects, the central region of the ASO comprises 8 to 10 contiguous phosphorothioate-linked DNA nucleotides, 8 to 10 contiguous mesyl phosphoroamidate-linked DNA nucleotides, or 8 to 10 DNA nucleotides linked by one or more phosphorothioate internucleoside linkages and one or more mesyl phosphoroamidate internucleoside linkages.

In some embodiments of any of the foregoing aspects, the ASO comprises at least one modified nucleotide having the following structure

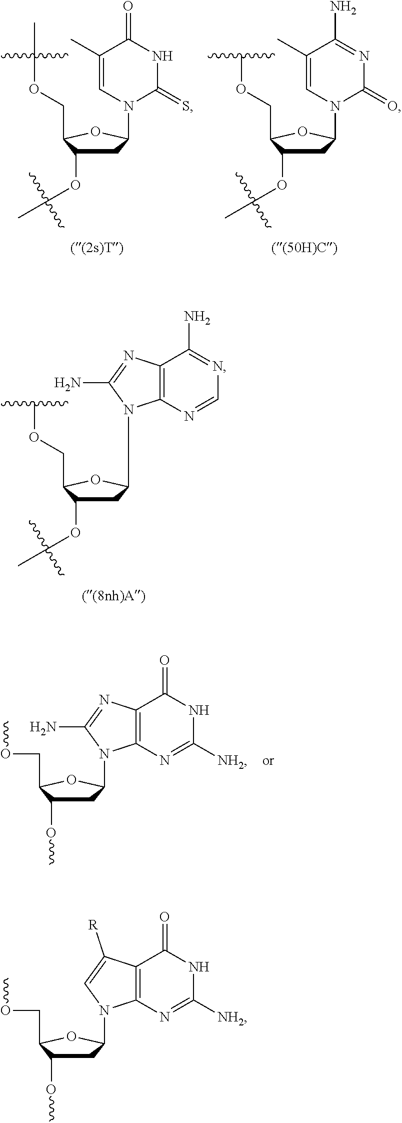

wherein:
R is a halogen or R′—C≡C—; and
R′ is C₆₋₁₂ aryl, 5- to 12-membered heteroaryl, hydroxy-C₁₋₆ alkyl, or C₁₋₇ alkanoyloxy.

In some embodiments of any of the foregoing aspects, the ASO comprises at least one modified nucleotide having the structure of:

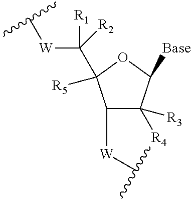

wherein:
W is independently O, N, or S;
R₁, R₂, and R₅ are independently H or D;
R₃ is H or F;
R₄ is F or OCH₃; and
Base is

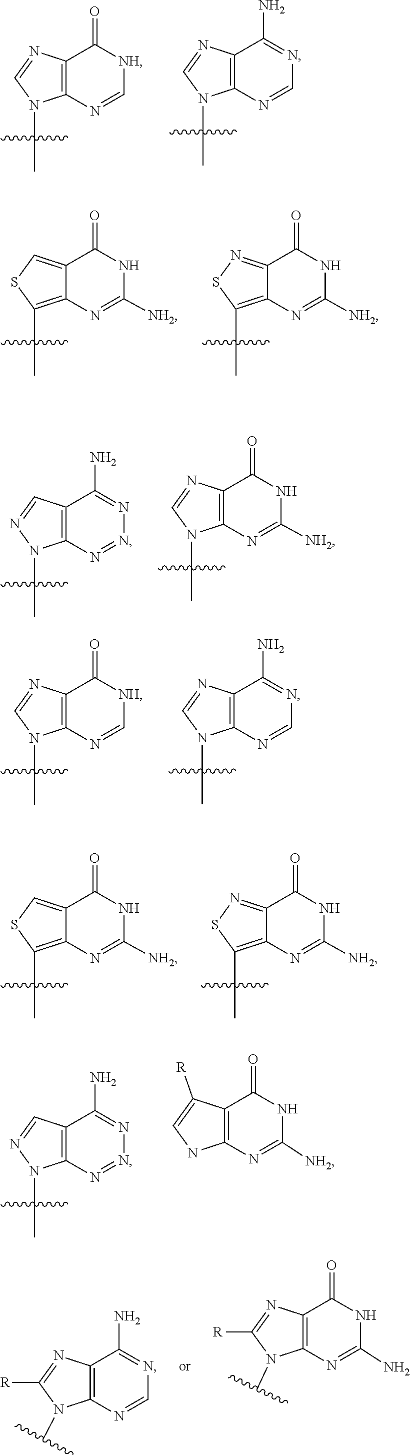

wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy.

In another aspect, the present disclosure provided an ASO molecule as shown in Table 1.

In another aspect, the present disclosure provides a pharmaceutical composition comprising the ASO molecule as disclosed here (e.g., any of the foregoing aspects or embodiments); and a pharmaceutically acceptable excipient.

In some embodiments, the pharmaceutical composition may further comprise 2, 3, 4, 5, 6, 7, 8, 9, 10 or more ASO molecules as disclosed herein.

In some embodiments, the pharmaceutical composition may further comprise an additional treatment agent. In some embodiments, the additional treatment agent is selected from a nucleotide analog, nucleoside analog, a capsid assembly modulator (CAM), a recombinant interferon, an entry inhibitor, a small molecule immunomodulatory, and oligonucleotide therapy, wherein the oligonucleotide therapy is optionally selected from an additional antisense oligonucleotide (ASO), a short interfering nucleic acid (siNA), NAPs, or STOPS™.

In another aspect, the present disclosure provides methods of treating a subject having a Hepatitis B virus (HBV) infection, comprising administering to the subject with HBV an ASO or a pharmaceutical composition as disclosed here (e.g., any of the foregoing aspects or embodiments).

In some embodiments, the methods may further comprise administering an additional therapeutic agent. In some embodiments, the additional treatment agent is selected from a nucleotide analog, nucleoside analog, a capsid assembly modulator (CAM), a recombinant interferon, an entry inhibitor, a small molecule immunomodulatory, and oligonucleotide therapy, wherein the oligonucleotide therapy is optionally selected from an additional antisense oligonucleotide (ASO), a short interfering nucleic acid (siNA), NAPs, or STOPS™. In some embodiments, the additional therapeutic agent is selected from the group consisting of include ALG-010133, ALG-000184, recombinant interferon alpha 2b, IFN-α, PEG-IFN-α-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, DCR-HBVS, JNJ-6379, GLS4, ABI-HO731, JNJ-440, NZ-4, RG7907, EDP-514, AB-423, AB-506, ABI-H03733 and ABI-H2158. In some embodiments, the ASO and the additional therapeutic agent are administered concurrently or consecutively.

In some embodiments, the treatment comprises reducing a viral load of HBV in the subject, reducing a level of a virus antigen in the subject, or a combination thereof.

In another aspect, the present disclosure provides methods of decreasing expression of a target gene in a subject, comprising administering to the an ASO or a pharmaceutical composition as disclosed here. In some embodiments, the target gene is a gene that is endogenous to the subject or the target gene is not endogenous to the subject. In some embodiments, the subject has a disease selected from hepatitis B virus (HBV), a coronavirus infection, and a liver disease, wherein the liver disease is, optionally, selected from nonalcoholic fatty liver disease (NAFLD), nonalcoholic steatohepatitis (NASH), and hepatocellular carcinoma (HCC).

In some embodiments of the disclosed methods, the subject is a mammal, optionally an adult human.

In some embodiments of the disclosed methods, the ASO is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg 14 mg/kg, or 15 mg/kg.

In some embodiments of the disclosed methods, the ASO is administered at a dose of between 0.5 mg/kg to 50 mg/kg, 0.5 mg/kg to 40 mg/kg 0.5 mg/kg to 30 mg/kg, 1 mg/kg to 50 mg/kg, 1 mg/kg to 40 mg/kg, 1 mg/kg to 30 mg/kg, 1 mg/kg to 20 mg/kg, 3 mg/kg to 50 mg/kg, 3 mg/kg to 40 mg/kg, 3 mg/kg to 30 mg/kg, 3 mg/kg to 20 mg/kg, 3 mg/kg to 15 mg/kg, 3 mg/kg to 10 mg/kg, 4 mg/kg to 50 mg/kg, 4 mg/kg to 40 mg/kg, 4 mg/kg to 30 mg/kg, 4 mg/kg to 20 mg/kg, 4 mg/kg to 15 mg/kg, 4 mg/kg to 10 mg/kg, 5 mg/kg to 50 mg/kg, 5 mg/kg to 40 mg/kg, 5 mg/kg to 30 mg/kg, 5 mg/kg to 20 mg/kg, 5 mg/kg to 15 mg/kg, or 5 mg/kg to 10 mg/kg.

In some embodiments of the disclosed methods, the ASO is administered at least 1, 2, 3, 4, 5, 6, 7, 8, 9, or 10 times.

In some embodiments of the disclosed methods, the ASO is administered at least 1, 2, 3, 4, 5, 6, 7, 8, 9, or 10 times a day, at least 1, 2, 3, 4, 5, 6, 7, 8, 9, or 10 times a week, or at least 1, 2, 3, 4, 5, 6, 7, 8, 9, or 10 times a month.

In some embodiments of the disclosed methods, the ASO is administered at least once every 1, 2, 3, 4, 5, 6, 7, 8, 9, 10, 11, 12, 13, 14, 15, 16, 17, 18, 19, 20, or 21 days.

In some embodiments of the disclosed methods, the ASO is administered for a period of at least 1, 2, 3, 4, 5, 6, 7, 8, 9, 10, 11, 12, 13, 14, 15, 16, 17, 18, 19, 20, or 21 days, or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 51, 52, 53, 54, or 55 weeks.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

The FIGURE shows the improvement of in vivo potency of select ASOs over LNA-DNA-LNA parent. G01 is vehicle dosed at 5 mL/kg, SC; G02 is ASO 59, dosed once at 5 mg/kg, SC on Day 0; G-04 is ASO 84, dosed once at 5 mg/kg, SC on Day 0.

### DETAILED DESCRIPTION

The present disclosure is directed to modified antisense oligonucleotides and pharmaceutical compositions of modified antisense oligonucleotides. The present disclosure is also directed to methods of using and preparing the antisense oligonucleotides and pharmaceutical compositions.

Antisense Oligonucleotides (ASOs)

Compounds of the present disclosure include modified antisense oligonucleotides (ASO). In some embodiments, the ASO comprises 14-22 nucleotide units, e.g., 14, 15, 16, 17, 18, 19, 20, 21, or 22 nucleotide units. In some embodiments, the ASO is a gapmer that comprises three regions: a 5′-wing region (A′) comprising modified nucleotides; a central region (B′) comprising nucleotides of a different type from the wings, e.g., nucleotides capable of inducing RNase H cleavage; and a 3′-wing region (C′) comprising modified nucleotides.

The disclosed ASOs comprise (i) a modified nucleotide such as Gutb, Nmln, 5prnl, G-clamp, or a combination thereof, (ii) at least one mesyl phosphoroamidate internucleoside linkage (referred to in sequences as “yp” when R^{a} is a methyl group); or (iii) a combination thereof. The structures of Gutb, Nmln, 5prnl, and G-clamp are shown below, and the structure of the mesyl phosphoroamidate linker is:

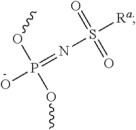

wherein R^{a} is C₁-C₆ alkyl, C₆₋₁₂ aryl, or a 5- to 12-membered heteroaryl.
In some embodiments, the structure of the mesyl phosphoroamidate linker is:

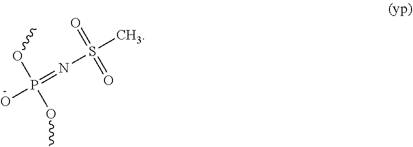

For example, the 5′-wing region and the 3′-wing regions can each independently comprise 2-6 nucleotides, e.g., 2, 3, 4, 5, or 6 nucleotides. One or more of these nucleotides can be modified (e.g., 1, 2, 3, 4, 5, or 6 of the nucleotides is modified). At least one of the modified nucleotides may comprise a structure of:

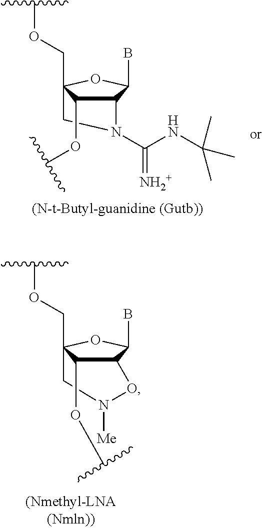

wherein B is a nucleobase. Additionally or alternatively, at least one of the modified nucleotides may comprise a structure of:

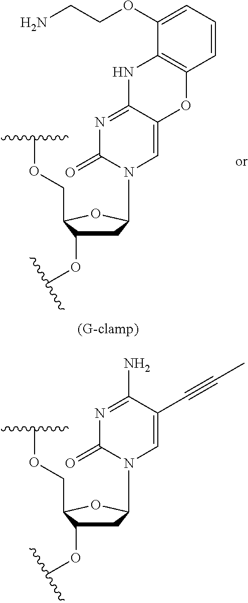

(5(Me)-propynl (5prnl)). Thus, the 5′-wing region and the 3′-wing regions can each independently comprise one or more of Gutb, Nmln, or both. Similarly, the 5′-wing region and the 3′-wing regions can each independently comprise one or more of G-clamp, 5prnl, or both; however, G-clamp and 5prnl are suitable for inclusion in the central region as well. For example, in some embodiments, (i) the central region (B′) comprises a modified nucleotide selected from G-clamp and 5prnl, (ii) the 5′-wing region (A′) comprises a modified nucleotide selected from Gutb and Nmln, (iii) the 3′-wing region (C′) comprises a modified nucleotide selected from Gutb and Nmln, or (iv) any combination thereof.

Additionally or alternatively, the central region may comprise 1, 2, 3, 4, 5 or more contiguous DNA nucleosides, linked by phosphodiester internucleoside linkages or thiophosphate (“ps”) internucleoside linkages. In other embodiments, the central region includes one or more modified nucleotide, mesyl phosphoroamidate (yp) internucleoside linkages, or a combination thereof. Further, the central region may include one or more modified nucleotide where the central region is capable of inducing RNase H cleavage. In some embodiments, the central region includes one or more modified nucleotide having a modified nucleobase. In some embodiments, the central region comprises 6, 7, 8, 9, 10, or 11 contiguous DNA nucleosides. In some embodiments, 1, 2, 3, 4, 5, 6, 7, 8, 9, 10, or 11 of the DNA nucleosides in the central region are modified. At least one of the modified nucleotides may comprise a structure of:

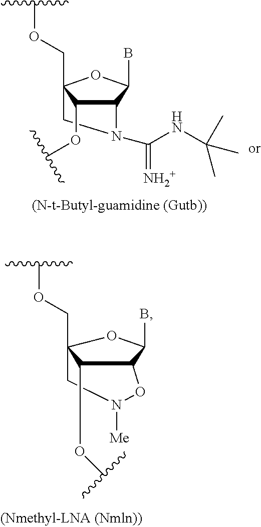

wherein B is a nucleobase. Additionally or alternatively, at least one of the modified nucleotides may comprise a structure of:

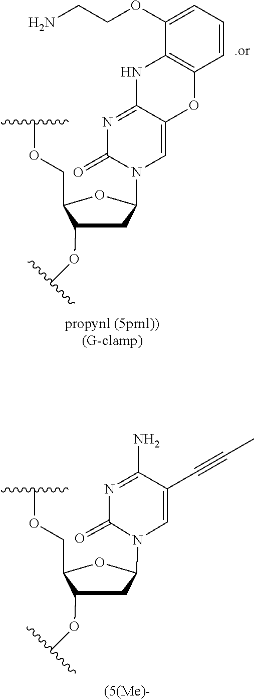

For the purposes of the present disclosure, the disclosed ASOs may comprise at least 1, at least 2, at least 3, at least 4, or at least 5 or more of Gutb, Nmln, 5prnl, G-clamp, or a combination thereof. Gutb, Nmln, 5prnl, G-clamp, or a combination thereof may be incorporated into the central region, the wing regions, or both. In general the modified locked nucleotides (Gutb and Nmln) are suitable for inclusion in the wing regions, whereas 5prnl and G-clamp are suitable for inclusion throughout the ASO or specifically in the central region. Additionally or alternatively, the disclosed ASOs may comprise 1, 2, 3, 4, 5, 6, 7, 8, 9, 10, 11, 12, 13, 14, 15, or more mesyl phosphoroamidate (yp) internucleoside linkages.

In some aspects, the gapmer ASO compounds of the disclosure include compounds of formula (I):

A′-B′-C′,

wherein A′ and C′ each independently comprise 2-6 nucleotides, with one or more being a modified nucleotide, B′ comprises 6 or more contiguous DNA nucleosides linked by phosphodiester or thiophosphate internucleoside linkages. In some embodiments, B′ comprises one or more modified DNA nucleosides. In some embodiments, the modified nucleotide is selected from locked nucleosides or 2′-substituted nucleosides. In some embodiments, the modified DNA nucleoside is selected from locked nucleosides or 2′-substituted nucleosides.

The number of nucleotides and/or nucleosides in A′, B′, and C′ can be selected from the following group (A′:B′:C′): (2:10:2), (2:10:3), (2:10:4), (2:10:5), (3:10:2), (3:10:3), (3:10:4), (3:10:5), (4:10:2), (4:10:3), (4:10:4), (4:10:5), (5:10:2), (5:10:3), (5:10:4), (5:10:5), (2:9:2), (2:9:3), (2:9:4), (2:9:5), (3:9:2), (3:9:3), (3:9:4), (3:9:5), (4:9:2), (4:9:3), (4:9:4), (4:9:5), (5:9:2), (5:9:3), (5:9:4), (5:9:5), (2:8:2), (2:8:3), (2:8:4), (2:8:5), (3:8:2), (3:8:3), (3:8:4), (3:8:5), (4:8:2), (4:8:3), (4:8:4), (4:8:5), (5:8:2), (5:8:3), (5:8:4), (5:8:5), (2:7:2), (2:7:3), (2:7:4), (2:7:5), (3:7:2), (3:7:3), (3:7:4), (3:7:5), (4:7:2), (4:7:3), (4:7:4), (4:7:5), (5:7:2), (5:7:3), (5:7:4), (5:7:5), (2:6:2), (2:6:3), (2:6:4), (2:6:5), (3:6:2), (3:6:3), (3:6:4), (3:6:5), (4:6:2), (4:6:3), (4:6:4), (4:6:5), (5:6:2), (5:6:3), (5:6:4), and (5:6:5).

In some embodiments, the 5′-wing region comprises one or more locked nucleosides or 2′-substituted nucleosides. In some embodiments, the 3′-wing region comprises one or more locked nucleosides or 2′-substituted nucleosides. In some embodiments, the central region comprises one or more locked nucleosides or 2′-substituted nucleosides. In some embodiments, the 5′-wing region, the 3′-wing region, the central region, or a combination thereof comprises one or more (e.g., 1, 2, 3, 4, 5, 6, 7, 8, 9, 10, 11, 12, 13, 14, 15) locked nucleosides or 2′-substituted nucleosides. The locked nucleoside can contain a bridge between the 4′ and the 2′ position of the sugar wherein the bridges comprises 2 to 4 optionally substituted atoms. For example, LNA nucleoside is:

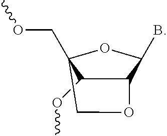

Other exemplary locked nucleosides include the following:

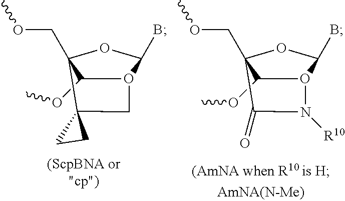

when R¹⁰ is CH₃, AmNA(N-alkyl) when R¹⁰ is C₁-C₆ alkyl);

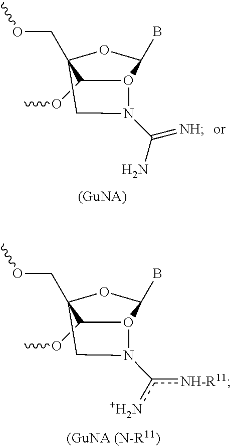

wherein B is a nucleobase; R¹⁰ is H or C₁-C₆ alkyl; and R¹¹ is C₁-C₆ alkyl. In certain embodiments, all nucleosides in the 5′-wing region are locked nucleosides. In some embodiments, all nucleosides in the 3′-wing region are locked nucleosides. In some embodiments, the 3′-wing region comprises LNA and one or two nucleosides selected from ScpBNA, AmNA, and GuNA. In some embodiments, 5′-wing region are all LNA and the 3′-wing region contains LNA and one or two nucleosides selected from ScpBNA, AmNA, and GuNA. Other nucleotides are included in PCT/JP2010/068409, PCT/JP2013/075370, PCT/JP2015/054308, PCT/JP2018/006061, and/or PCT/JP2018/006062, which are incorporated by reference in their entirety. Gutb and Nmln are additional examples of locked nucleotides that may be included in the 5′-wing region or the 3′-wing region or both.

In some embodiments, the 5′-wing region of an ASO comprises 2 to 6 phosphorothioate-linked locked nucleosides, mesyl phosphoroamidate-linked locked nucleosides, or a combination thereof. In some embodiments, the 5′-wing region comprises 2 to 6 phosphorothioate-linked 2′ substituted nucleosides, mesyl phosphoroamidate-linked 2′ substituted nucleosides, or a combination thereof. In some embodiments, the 5′-wing region comprises at least one locked nucleoside and at least one 2′ substituted nucleoside, wherein the locked nucleoside and the 2′ substituted nucleoside are linked by a phosphorothioate linker or a mesyl phosphoroamidate linker. In some embodiments, the 5′-wing region further comprises a RNA nucleoside or DNA nucleoside, wherein the RNA nucleoside and DNA nucleoside are not locked nucleosides or 2′-substituted nucleosides. In some embodiments, at least two nucleosides of the 5′-wing region are linked by a phosphorothioate linker or a mesyl phosphoroamidate linker. In some embodiments, at least 2, 3, 4, 5, or 6 nucleosides of the 5′-wing region are linked by a phosphorothioate linker, a mesyl phosphoroamidate linker, or a combination thereof.

In some embodiments, the 3′-wing region of an ASO comprises 2 to 6 phosphorothioate-linked locked nucleosides, mesyl phosphoroamidate-linked locked nucleosides, or a combination thereof. In some embodiments, the 3′-wing region comprises 2 to 6 phosphorothioate-linked substituted nucleosides, mesyl phosphoroamidate-linked substituted nucleosides, or a combination thereof. In some embodiments, the 3′-wing region comprises at least one locked nucleoside and at least one 2′ substituted nucleoside, wherein the locked nucleoside and the 2′ substituted nucleoside are linked by a phosphorothioate linker or a mesyl phosphoroamidate linker. In some embodiments, the 3′-wing region further comprises a RNA nucleoside or DNA nucleoside, wherein the RNA nucleoside and DNA nucleoside are not locked nucleosides or 2′-substituted nucleosides. In some embodiments, at least two nucleosides of the 3′-wing region are linked by a phosphorothioate linker or a mesyl phosphoroamidate linker. In some embodiments, at least 2, 3, 4, 5, or 6 nucleosides of the 3′-wing region are linked by a phosphorothioate linker, a mesyl phosphoroamidate linker, or a combination thereof.

In some embodiments, one or more of the nucleotides in the 5′-wing region and/or the 3′-wing region comprises a thiophosphate internucleoside linkage or a mesyl phosphoroamidate internucleoside linkage. In some embodiments, all nucleotides in the 5′-wing region comprises a thiophosphate internucleoside linkage. In some embodiments, all nucleotides in the 3′-wing region comprises a thiophosphate internucleoside linkage. In some embodiments, all nucleotides in the 5′-wing region comprises a mesyl phosphoroamidate internucleoside linkage. In some embodiments, all nucleotides in the 3′-wing region comprises a mesyl phosphoroamidate internucleoside linkage.

In some embodiments, the central region includes one or more modified nucleotide having a modified nucleobase. For example, the central region can include at least 1, at least 2, at least 3, at least 4, or at least 5 or more of Gutb, Nmln, 5prnl, G-clamp, or a combination thereof. In some embodiments, the central region comprises at least 1, at least 2, at least 3, at least 4, or at least 5 or more of 5prnl, G-clamp, or a combination thereof. Additionally or alternatively, the central region can include one or more modified nucleotide having the following structure:

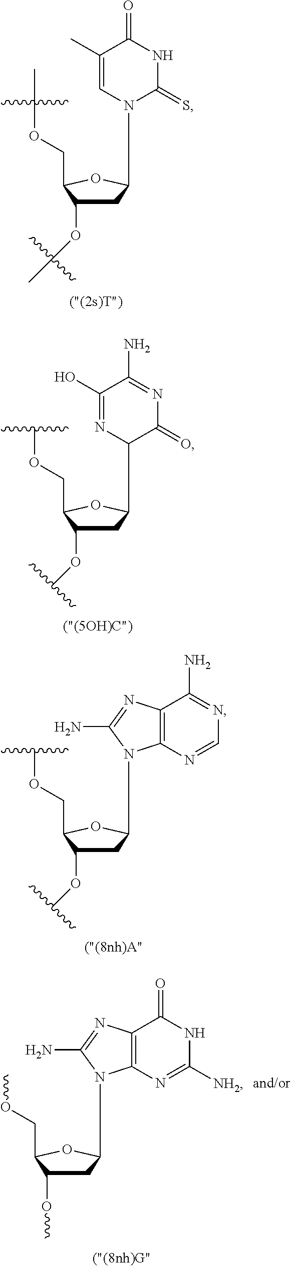

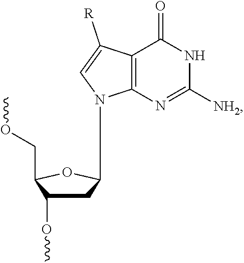

where R is a halogen or R′—C≡C—; and R′ is C₆₋₁₂ aryl, 5- to 12-membered heteroaryl, hydroxy-C₁₋₆ alkyl, or C₁₋₇ alkanoyloxy. In some embodiments, the central region includes one modified nucleotide (e.g., (2s)T or (5OH)C) at the 1^{st}, 2^{nd}, 3^{rd} or 4^{th} gap nucleoside position (from the 5′ end). In some embodiments, the modified nucleotide is at the 3^{rd} gap nucleoside position (from the 5′ end). In some embodiments, the modified nucleotide is a nucleotide having the structure of:

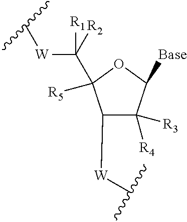

wherein:
W is independently O, N, or S;
R₁, R₂, and R₅ are independently H or D;
R₃ is H or F;
R₄ is F or OCH₃; and
Base is

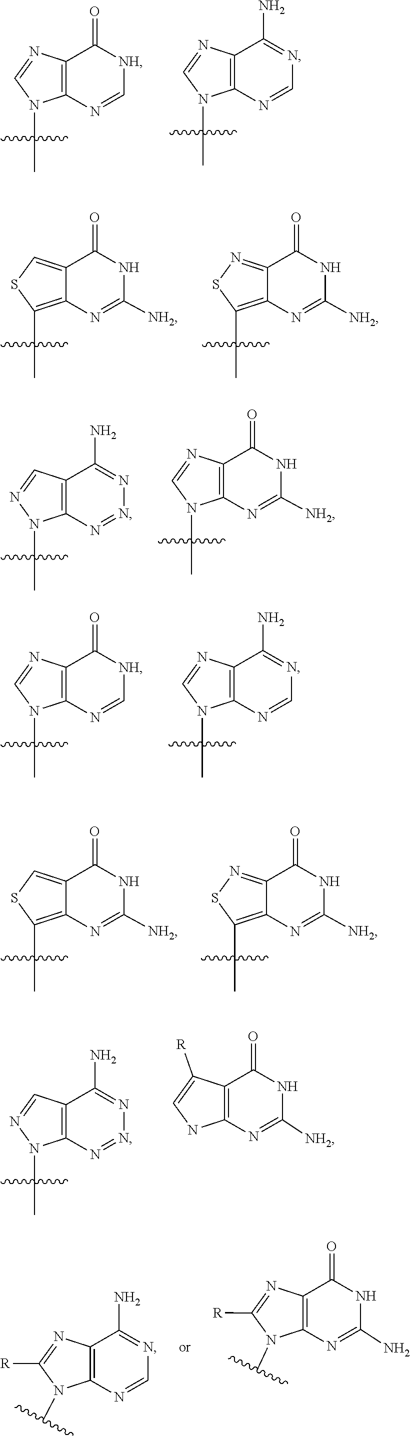

wherein:
R is a halogen or R′—C≡C—; and
R′ represents C₆₋₁₂ aryl, 5- to 12-membered heteroaryl, hydroxy-C₁₋₆ alkyl, or C₁₋₇ alkanoyloxy. In some embodiments, C₁₋₇ alkanoyl includes, but is not limited to. formyl, acetyl, ethyl carbonyl, n-propyl carbonyl, isopropyl carbonyl, n-butyl carbonyl, isobutyl carbonyl, t-butyl carbonyl, n-pentyl carbonyl, and n-hexyl carbonyl. Other modified nucleotides include those in PCT/JP2018/006061, which is incorporated by reference in its entirety.

As used herein, unless otherwise indicated, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Examples of aryl groups include, without limitation, the radicals of benzene, naphthalene, and azulene.

As used herein, unless otherwise indicated, “heteroaryl” refers to a ring that has a fully delocalized pi-electron system and contains one or more heteroatoms (e.g., one to three heteroatoms, or one to four heteroatoms, or one to five heteroatoms) independently selected from the group consisting of nitrogen, oxygen, and sulfur in the ring. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, pyrrole, oxazole, thiazole, imidazole, pyrazole, isoxazole, isothiazole, triazole, thiadiazole, pyridine, pyridazine, pyrimidine, pyrazine, and triazine.

In some embodiments, the central region of an ASO comprises at least 5 contiguous phosphorothioate-linked DNA nucleosides, at least 5 contiguous mesyl phosphoroamidate-linked DNA nucleosides, or a combination thereof. In some embodiments, at least 2, 3, 4, 5, or 6 nucleosides of the central region are linked by a phosphorothioate linker, a mesyl phosphoroamidate linker, or a combination thereof. In some embodiments, a DNA nucleoside of central region is linked to a nucleoside of a 5′-wing region by a phosphorothioate linker or a mesyl phosphoroamidate linker. In some embodiments, a DNA nucleoside of central region is linked to a nucleoside of a 3′-wing region by a phosphorothioate linker or a mesyl phosphoroamidate linker. In some embodiments, the central region comprises 8 to 10 contiguous phosphorothioate-linked DNA nucleosides, 8-10 contiguous mesyl phosphoroamidate-linked DNA nucleosides, or a combination thereof.

In some embodiments, the ASO is complementary or hybridizes to a viral target RNA sequence that begins in an X region of HBV or in an S region of HBV. The vital target may, e.g., begin at the 5′-end of target-site in acc. KC315400.1 (genotype B, “gt B”), or in any one of genotypes A, C, or D. The skilled person would understand the HBV position, e.g., as described in Wing-Kin Sung, et al., Nature Genetics 44:765 (2012). In some embodiments, the S region is defined as from the beginning of small S protein (in genotype B KC315400.1 isolate, position #155) to before beginning of X protein (in genotype B KC315400.1 isolate, position #1373). In some embodiments, the X region is defined as from the beginning X protein (in genotype B KC315400.1 isolate, position #1374) to end of DR2 site (in genotype B KC315400.1 isolate, position #1603).

In some embodiments, the ASO is complementary or hybridizes to a viral target RNA sequence that comprises, consists of, or consists essentially of at least 5, 6, 7, 8, 9, 10, 11, 12, 13, 14, or 15 contiguous nucleotides within positions 100-800 or 1050-1700 of SEQ ID NO: 89. In some embodiments, the ASO is complementary or hybridizes to a viral target RNA sequence that comprises, consists of, or consists essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within positions 100-800 or 1050-1700 of SEQ ID NO: 89. In some embodiments, the ASO is complementary or hybridizes to a viral target RNA sequence that comprises, consists of, or consists essentially of at least 5, 6, 7, 8, 9, 10, 11, 12, 13, 14, or 15 contiguous nucleotides within positions 180-280, 300 to 450, 650 to 775, 1125 to 1300, or 1400 to 1650 of SEQ ID NO: 89. In some embodiments, the ASO is complementary or hybridizes to a viral target RNA sequence that comprises, consists of, or consists essentially of at least 5, 6, 7, 8, 9, 10, 11, 12, 13, 14, or 15 contiguous nucleotides within positions 180 to 215, 230 to 270, 350 to 420, 675 to 730, 1165 to 1210, 1245 to 1290, 1400 to 1480, or 1500 to 1630 of SEQ ID NO: 89. In some embodiments, the ASO is complementary or hybridizes to a viral target RNA sequence that comprises, consists of, or consists essentially of at least 5, 6, 7, 8, 9, 10, 11, 12, 13, 14, or 15 contiguous starting at position 191, 245, 246, 276, 376, 377, 381, 383, 694, 700, 1182, 1261, 1262, 1408, 1410, 1426, 1431, 1432, 1433, 1435, 1438, 1441, 1443, 1513, 1516, 1517, 1518, 1519, 1520, 1521, 1522, 1527, 1559, 1575, 1576, 1577, 1580, 1581, 1582, or 1589 of SEQ ID NO: 89. In some embodiments, the ASO is perfectly complementary to the viral target RNA sequence. In some embodiments, there is less than or equal to 5, 4, 3, 2, or 1 mismatches between the ASO and the viral target sequence. In some embodiments, there is less than or equal to 2 mismatches between the ASO and the viral target sequence. In some embodiments, there is less than or equal to 1 mismatch between the ASO and the viral target sequence. In some embodiments, the mismatch is in the wing region of the ASO. In some embodiments, the mismatch is in the 5′ wing region of the ASO. In some embodiments, the mismatch is in the 3′ wing region of the ASO. In some embodiments, the mismatch is in the central region of the ASO.

In some embodiments, the central region is complementary or hybridizes to a viral target RNA sequence that comprises, consists of, or consists essentially of at least 5, 6, 7, 8, 9, 10, 11, 12, 13, 14, or 15 contiguous nucleotides within positions 100-800 or 1050-1700 of SEQ ID NO: 89. In some embodiments, the central region is complementary or hybridizes to a viral target RNA sequence that comprises, consists of, or consists essentially of 5 to 15, 5 to 14, 5 to 13, 5 to 12, 5 to 11, 5 to 10, 5 to 9, 5 to 8, 6 to 15, 6 to 14, 6 to 13, 6 to 12, 6 to 11, 6 to 10, 7 to 15, 7 to 14, 7 to 13, 7 to 12, or 7 to 11 contiguous nucleotides within positions 100-800 or 1050-1700 of SEQ ID NO: 89. In some embodiments, the central region is complementary or hybridizes to a viral target RNA sequence that comprises, consists of, or consists essentially of at least 5, 6, 7, 8, 9, 10, 11, 12, 13, 14, or 15 contiguous nucleotides within positions 180-280, 300 to 450, 650 to 775, 1125 to 1300, or 1400 to 1650 of SEQ ID NO: 89. In some embodiments, the central region is complementary or hybridizes to a viral target RNA sequence that comprises, consists of, or consists essentially of at least 5, 6, 7, 8, 9, 10, 11, 12, 13, 14, or 15 contiguous nucleotides within positions 180 to 215, 230 to 270, 350 to 420, 675 to 730, 1165 to 1210, 1245 to 1290, 1400 to 1480, or 1500 to 1630 of SEQ ID NO: 89. In some embodiments, the central region is complementary or hybridizes to a viral target RNA sequence that comprises, consists of, or consists essentially of at least 5, 6, 7, 8, 9, 10, 11, 12, 13, 14, or 15 contiguous starting at position 191, 245, 246, 276, 376, 377, 381, 383, 694, 700, 1182, 1261, 1262, 1408, 1410, 1426, 1431, 1432, 1433, 1435, 1438, 1441, 1443, 1513, 1516, 1517, 1518, 1519, 1520, 1521, 1522, 1527, 1559, 1575, 1576, 1577, 1580, 1581, 1582, or 1589 of SEQ ID NO: 89. In some embodiments, the central region is perfectly complementary to the viral target RNA sequence. In some embodiments, there is less than or equal to 5, 4, 3, 2, or 1 mismatches between the central region and the viral target sequence. In some embodiments, there is less than or equal to 2 mismatches between the central region and the viral target sequence. In some embodiments, there is less than or equal to 1 mismatch between the central region and the viral target sequence.

In some embodiments, the ASO comprises a nucleotide sequence that is at least 90%, at least 91%, at least 92%, at least 93%, at least 94%, at least 95%, at least 96%, at least 97%, at least 98%, or 100% identical to a nucleotide sequence selected from the sequences listed in Table 1.

In some embodiments, the ASOs of the disclosure may have a sequence that differs from an ASO of Table 1 by one nucleoside. In some embodiments, the ASOs of the disclosure may have a sequence that differs from an ASO of Table 1 by two nucleosides. In some embodiments, the ASOs of the disclosure may have a sequence that differs from an ASO of Table 1 by three nucleosides. In some embodiments, the ASOs of the disclosure may have a sequence that differs from an ASO of Table 1 by four nucleosides.

In some embodiments, the ASOs of the disclosure may have a sequence of Table 1, but one T in the central region is replaced by (2s)T, one C in the central region is replaced by (5OH)C, and/or one A is replaced by (8nh)A in the central region. In some embodiments, the ASOs of the disclosure may have a sequence of Table 1, but with one or two ScpBNA, AmNA, or GuNA in the 5′ wing portion. In some embodiments, the ASOs of the disclosure may have a sequence of Table 1, but with one or two ScpBNA, AmNA, or GuNA in the 3′ wing portion. In some embodiments, the ASOs of the disclosure may have a sequence of Table 1, but with a mA or mU appended to the 5′ end of the sequence. In some embodiments, the ASOs of the disclosure may have a sequence of Table 1, but with a mA or mU appended to the 5′ end of the sequence, the 3′ end of the sequence, or both that links to a GalNAc derivative (e.g., GalNAc4, such as GalNAc4-(PS)2-p-, or GalNAc6, such as GalNAc6-(PS)2-p-), as detailed herein.

In some embodiments, the ASO comprises a nucleotide sequence that is at least 90%, at least 91%, at least 92%, at least 93%, at least 94%, at least 95%, at least 96%, at least 97%, at least 98%, or 100% identical to a nucleotide sequence of any one of SEQ ID NOs: 1-88.

In some embodiments, the ASOs of the disclosure have a sequence that differs from any of the nucleotides of SEQ ID NOs: 1-88 by one nucleoside. In other embodiments, the ASO has a sequence that differs from any of the nucleotides of SEQ ID NOs: 1-88 by 1, 2, 3 or 4 nucleosides. In some embodiments, the ASOs of the disclosure have a sequence of any one of SEQ ID NOs: 1-88, but one T in the central region is replaced by (2s)T, one C in the central region is replaced by (5OH)C, and/or one A is replaced by (8nh)A in the central region. In some embodiments, the ASOs of the disclosure have a sequence of any one of SEQ ID NOs: 1-88, but with one or two ScpBNA, AmNA, or GuNA in the 5′ wing portion. In some embodiments, the ASOs of the disclosure have a sequence of any one of SEQ ID NOs: 1-88, but with one or two ScpBNA, AmNA, or GuNA in the 3′ wing portion. In some embodiments, the ASOs of the disclosure have a sequence of any one of SEQ ID NOs: 1-88, but with a mA or mU appended to the 5′ end of the sequence. In some embodiments, the ASOs of the disclosure have a sequence of any one of SEQ ID NOs: 1-88, but with a mA or mU appended to the 5′ end of the sequence that links to a GalNAc derivative (e.g., GalNAc4, such as GalNAc4-(PS)2-p-, or GalNAc6, such as GalNAc6-(PS)2-p-), as detailed herein.

Target RNA Sequence

The disclosed ASO can decrease expression of a target RNA sequence (e.g., a target gene) by recruiting RNAse H to cleave and degrade the RNA transcript of the target RNA sequence, lowering RNA levels and thereby lowering levels of the protein encoded by the target RNA sequence.

For the purposes of the present disclosure, the target RNA sequence may be any gene in a cell. In some embodiments, the target gene is a viral gene. In some embodiments, the viral gene is from a DNA virus. In some embodiments, the DNA virus is a double-stranded DNA (dsDNA) virus. In some embodiments, the dsDNA virus is a hepadnavirus. In some embodiments, the hepadnavirus is a hepatitis B virus (HBV). In some embodiments, the HBV is selected from HBV genotypes A-J. In some embodiments, the viral disease is caused by an RNA virus. In some embodiments, the RNA virus is a single-stranded RNA virus (ssRNA virus). In some embodiments, the ssRNA virus is a positive-sense single-stranded RNA virus ((+)ssRNA virus). In some embodiments, the (+)ssRNA virus is a coronavirus. In some embodiments, the coronavirus is a β-coronaviruses. In some embodiments, the β-coronaviruses is selected from the group consisting of severe acute respiratory syndrome coronavirus 2 (SARS-CoV-2) (also known by the provisional name 2019 novel coronavirus, or 2019-nCoV), human coronavirus OC43 (hCoV-OC43), Middle East respiratory syndrome-related coronavirus (MERS-CoV, also known by the provisional name 2012 novel coronavirus, or 2012-nCoV), and severe acute respiratory syndrome-related coronavirus (SARS-CoV, also known as SARS-CoV-1). In some embodiments, the β-coronaviruses is SARS-CoV-2, the causative agent of COVID-19. Some exemplary target genes are shown in Table 6 at the end of the specification.

In some embodiments, the target RNA sequence is selected from the S gene or X gene of the HBV. In some embodiments, the HBV has a genome sequence shown in the nucleotide sequence of SEQ ID NO: 90 which corresponds to the nucleotide sequence of GenBank Accession No. U95551.1, which is incorporated by reference in its entirety.

An exemplary HBV genome sequence is shown in SEQ ID NO: 89, corresponding to Genbank Accession No. KC315400.1, which is incorporated by reference in its entirety. Nucleotides 2307 . . . 3215, 1 . . . 1623 of SEQ ID NO: 89 correspond to the polymerase/RT gene sequence, which encodes for the polymerase protein. Nucleotides 2848 . . . 3215, 1 . . . 835 of SEQ ID NO: 89 correspond to the PreS1/S2/S gene sequence, which encodes for the large S protein. Nucleotides 3205 . . . 3215, 1 . . . 835 of SEQ ID NO: 89 correspond to the PreS2/S gene sequence, which encodes for the middle S protein. Nucleotides 155 . . . 835 of SEQ ID NO: 89 correspond to the S gene sequence, which encodes the small S protein. Nucleotides 1374 . . . 1838 of SEQ ID NO: 89 correspond to the X gene sequence, which encodes the X protein. Nucleotides 1814 . . . 2452 of SEQ ID NO: 89 correspond to the PreC/C gene sequence, which encodes the precore/core protein. Nucleotides 1901 . . . 2452 of SEQ ID NO: 89 correspond to the C gene sequence, which encodes the core protein. The HBV genome further comprises viral regulatory elements, such as viral promoters (preS2, preS1, Core, and X) and enhancer elements (ENH1 and ENH2). Nucleotides 1624 . . . 1771 of SEQ ID NO: 89 correspond to ENH2. Nucleotides 1742 . . . 1849 of SEQ ID NO: 60 correspond to the Core promoter. Nucleotides 1818 . . . 3215, 1 . . . 1930 of SEQ ID NO: 89 correspond to the pregenomic RNA (pgRNA), which encodes the core and polymerase proteins.

In some embodiments, the target RNA sequence is selected from genome of SARS-CoV. In some embodiments, SARS-CoV has a genome corresponding to the nucleotide sequence of GenBank Accession No. NC_004718.3, which is incorporated by reference in its entirety.

In some embodiments, the target RNA sequence is selected from the genome of MERS-CoV. In some embodiments, MERS-CoV has a genome corresponding to the nucleotide sequence of GenBank Accession No. NC_019843.3, which is incorporated by reference in its entirety.

In some embodiments, the target RNA sequence is selected from the genome of hCoV-OC43. In some embodiments, hCoV-OC43 has a genome corresponding to the nucleotide sequence of GenBank Accession No. NC_006213.1, which is incorporated by reference in its entirety.

In some embodiments, the target RNA sequence is selected from genome of SARS-CoV-2. In some embodiments, SARS-CoV-2 has a genome sequence corresponding to the nucleotide sequence of GenBank Accession No. NC_045512.2, which is incorporated by reference in its entirety.

In some embodiments, the target RNA sequence may be any hydroxysteroid dehydrogenase gene. In any embodiment, the gene is hydroxysteroid 17-beta dehydrogenase 13 (HSD17B13). The HSD17B13 has a sequence shown in the nucleotide sequence of SEQ ID NO: 91, which corresponds to the nucleotide sequence of the coding sequence of GenBank Accession No. NM_178135.5 (nucleotides 42 to 944), which is incorporated by reference in its entirety.

In some embodiments, the target RNA sequence is at least about 60%, 65%, 70%, 75%, 80%, 85%, 90%, 95%, or 100% identical to a nucleotide region within SEQ ID NO: 91, with the exception that the thymines (Ts) in SEQ ID NO: 91 are replaced with uracil (U). In some embodiments, the first nucleotide sequence is at least about 60%, 65%, 70%, 75%, 80%, 85%, 90%, 95%, or 100% identical to 15 to 30, 15 to 25, 15 to 23, 15 to 22, 15 to 21, 17 to 25, 17 to 23, 17 to 22, 17 to 21, or 19 to 21 nucleotides within SEQ ID NO: 91.

In some embodiments, the target RNA sequence is involved in liver metabolism. In some embodiments, the target RNA sequence is an inhibitor of the electron transport chain. In some embodiments, the target gene encodes the MCJ protein (MCJ/DnaJC15 or Methylation-Controlled J protein). In some embodiments, the MCJ protein is encoded by the mRNA sequence of SEQ ID NO: 92, which corresponds to the nucleotide sequence of GenBank Accession No. NM_013238.3, which is incorporated by reference in its entirety.

In some embodiments, the target RNA sequence is TAZ. In some embodiments, TAZ comprises the nucleotide sequence of SEQ ID NO: 93, which corresponds to the nucleotide sequence of GenBank Accession No. NM_000116.5, which is incorporated by reference in its entirety.

In some embodiments, the target RNA sequence is angiopoietin like 3 (ANGPTL3). In some embodiments, ANGPTL3 comprises the nucleotide sequence of SEQ ID NO: 94, which corresponds to the nucleotide sequence of GenBank Accession No. NM_014495.4, which is incorporated by reference in its entirety.

In some embodiments, the target RNA sequence is diacylglycerol acyltransferase 2 (DGAT2). In some embodiments, DGAT2 comprises the nucleotide sequence of SEQ ID NO: 95, which corresponds to the nucleotide sequence of GenBank Accession No. NM_001253891.1, which is incorporated by reference in its entirety.

Conjugated Moiety

The present disclosure is also directed to additional components conjugated to the ASO such as targeting moieties and oligonucleotides modified at one or more end. In some embodiments, the conjugated moiety is selected from galactosamine, peptides, proteins, sterols, lipids, phospholipids, biotin, phenoxazines, active drug substance, cholesterols, phenanthridine, anthraquinone, acridine, fluoresceins, rhodamines, coumarins, folate, and dyes.

In some embodiments, the targeting moiety may comprise a carbohydrate, such as a monosaccharide, for example N-acetylgalactosamine (GalNAc), disaccharides, trisaccharides, tetrasaccharides, oligosaccharides, and polysaccharides. In some embodiments, the targeting moiety one or more GalNAc derivatives, such as two or three GalNAc derivatives attached to the ASO through one or more linkers, optionally in a consecutive structure. In certain embodiments, the targeting moiety comprises three consecutive GalNAc moieties attached through linkers, such as:

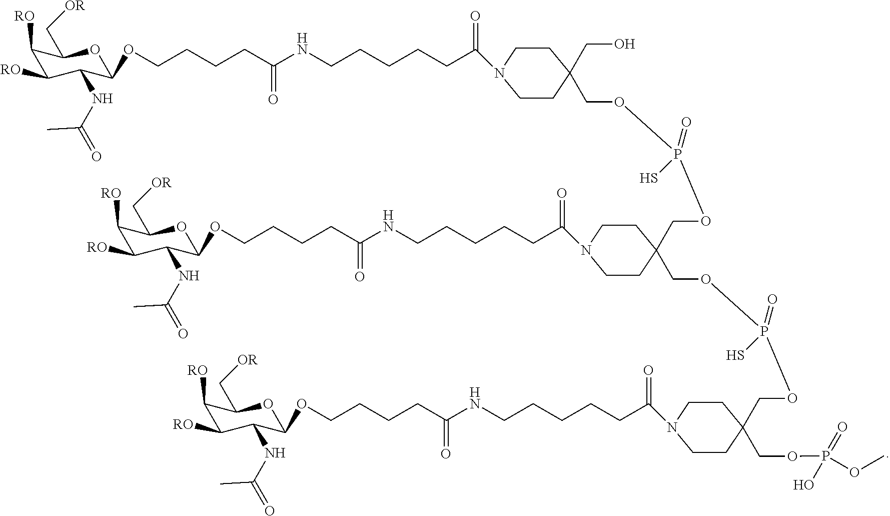

In some embodiments, the conjugated moiety is galactosamine. In some embodiments, any of the ASOs disclosed herein are attached to a conjugated moiety that is galactosamine. In some embodiments, the galactosamine is N-acetylgalactosamine (GalNAc). In some embodiments, any of the ASOs disclosed herein comprise GalNAc. In some embodiments, the GalNAc is of Formula (VI):

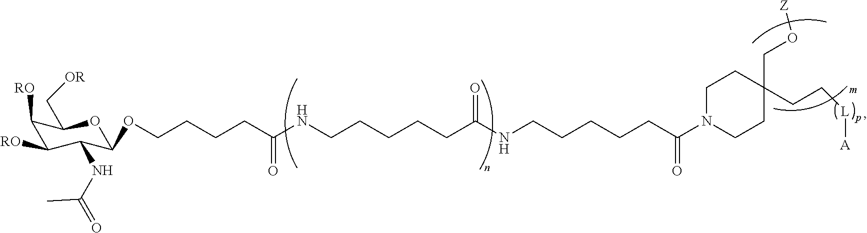

wherein m is 1, 2, 3, 4, or 5; each n is independently 1 or 2; p is 0 or 1; each R is independently H or a first protecting group; each Y is independently selected from —O—P(═O)(SH)—, —O—P(═O)(O)—, —O—P(═O)(OH)—, —O—P(S)S—, and —O—; Z is H or a second protecting group; either L is a linker or L and Y in combination are a linker; and A is H, OH, a third protecting group, an activated group, or an oligonucleotide. In some embodiments, the first protecting group is acetyl. In some embodiments, the second protecting group is trimethoxytrityl (TMT). In some embodiments, the activated group is a phosphoramidite group. In some embodiments, the phosphoramidite group is a cyanoethoxy N,N-diisopropylphosphoramidite group. In some embodiments, the linker is a C6-NH₂ group. In some embodiments, A is an ASO. In some embodiments, R is H, Z is H, and n is 1. In some embodiments, R is H, Z is H, and n is 2.

In some embodiments, the GalNAc is Formula (VII):

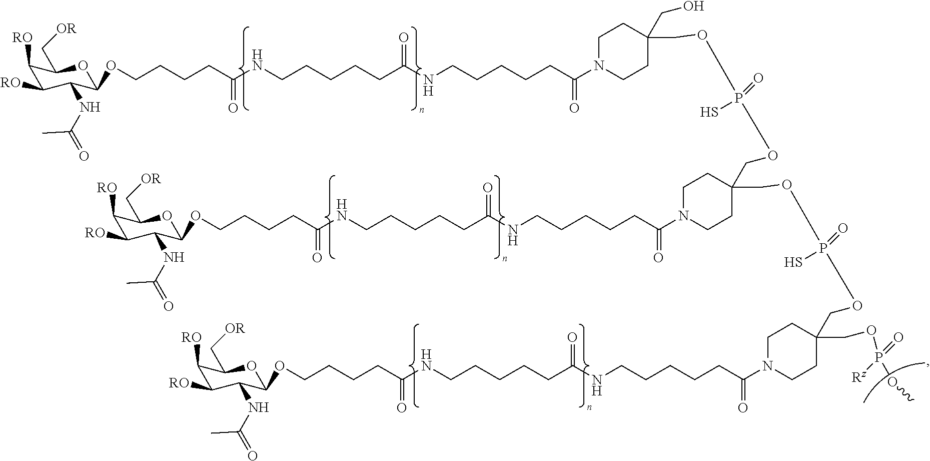

wherein R^{z} is OH or SH; and each n is independently 1 or 2. In some embodiments, the targeting ligand may be a GalNAc targeting ligand may comprise 1, 2, 3, 4, 5 or 6 GalNAc units. In some embodiments, the targeting ligand may be a GalNAc selected from GalNAc2, GalNAc3, GalNAc4, GalNAc5, and GalNAc6.

In some embodiments, the GalNAc may be GalNAc amidite, GalNAc 4 CPG, GalNAc phophoramidite, or GalNAc4-ps-GalNAc4-ps-GalNAc4. These GalNAc moieties are shown below:

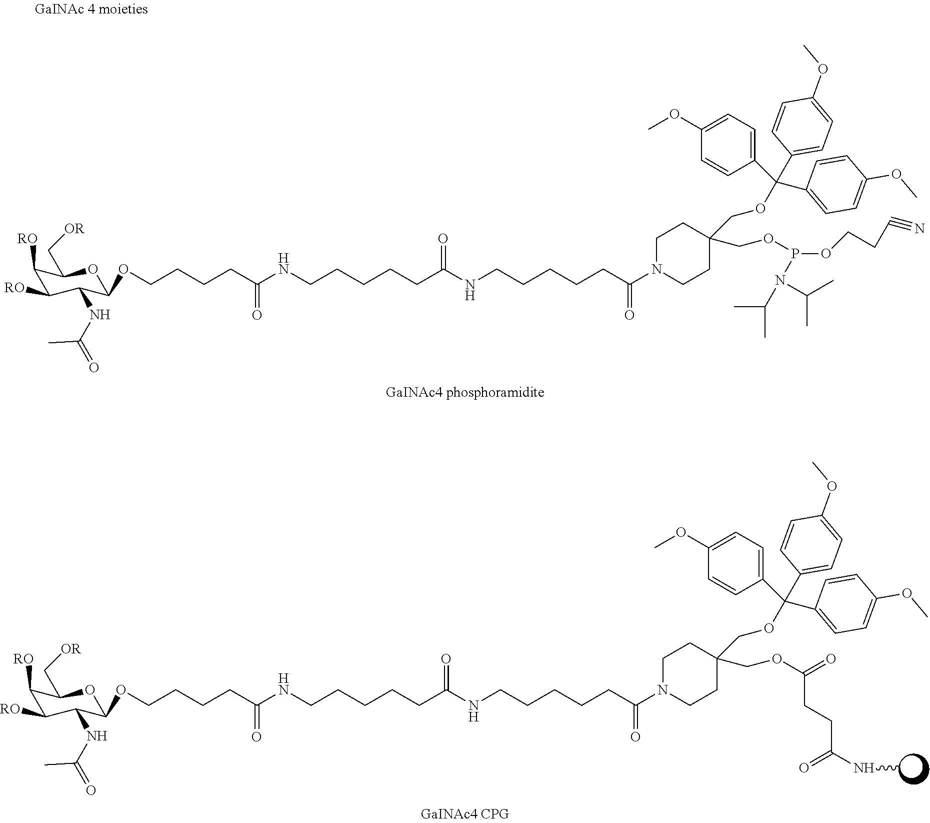

GalNAc3, GalNAc4, GalNAc5 and GalNAc6 may be conjugated to an ASO disclosed herein during synthesis with 1 2, or 3 moieties. Further GalNAc moieties, such as GalNAc1 and GalNAc2, can be used to form 5′ and 3′-GalNAc using post synthesis conjugation.

GalNAc Phosphoramidites

| GalNAc building blocks | After Attachment to Oligos (Nomenclature) |
|---|---|
| GalNAc-3 phosphoramidite 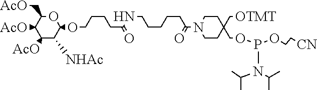 | 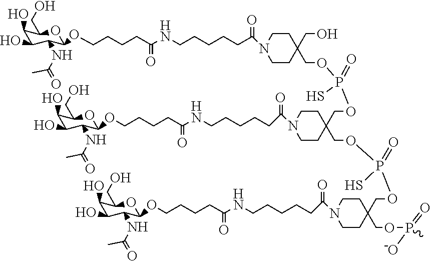 |
| | (GalNAc3-(PS)2-p) |
| GalNAc-4 phosphoramidite 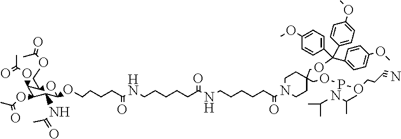 | 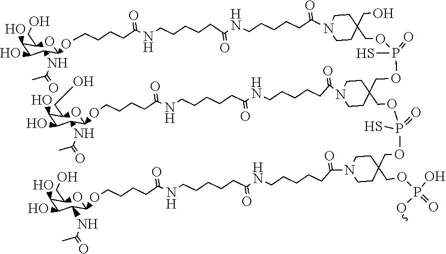 |
| | (GalNAc4-(PS)2-p) |
| GalNAc-5 phosphoramidite 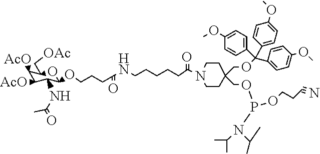 | 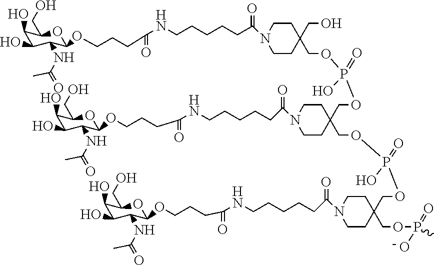 |
| | (GalNAc5-(PS)2-p) |
| GalNAc-6 phosphoramidite 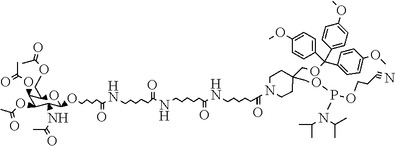 | 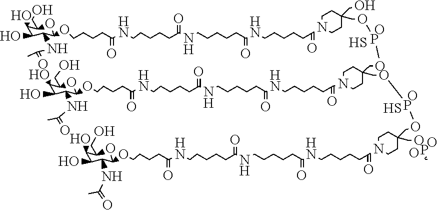 |
| | (GalNAc6-(PS)2-p) |

In some embodiments, the ASO contains a targeting moiety at the 5′-end, the 3′-end, or both ends of the ASO. The conjugated moiety may be attached to the ASO via 1, 2, 3, 4, or 5 or more linkers. In some embodiments, the one or more linkers are independently selected from the group consisting of a phosphodiester (p or po) linker, phosphorothioate (ps) linker, mesyl phosphoramidate linker (yp), phosphoramidite (HEG) linker, triethylene glycol (TEG) linker, and/or phosphorodithioate linker. In some embodiments, the one or more linkers are independently selected from the group consisting of p-(PS)2, (PS)2-p-TEG-p, (PS)2-p-HEG-p, and (PS)2-p-(HEG-p)2.

In some embodiments, the conjugated moiety is a lipid moiety. In some embodiments, any of the ASOs disclosed herein are attached to a conjugated moiety that is a lipid moiety. Examples of lipid moieties include, but are not limited to, a cholesterol moiety, a thioether, e.g., hexyl-S-tritylthiol, a thiocholesterol, an aliphatic chain, e.g., dodecandiol or undecyl residues a phospholipid, e.g., di-hexadecyl-rac-glycerol or triethylammonium 1-di-O-hexadecyl-rac-glycero-S—H-phosphonate, a polyamine or a polyethylene glycol chain, adamantane acetic acid, a palmityl moiety, or an octadecylamine or hexylamino-carbonyl-oxycholesterol moiety.

In some embodiments, the conjugated moiety is an active drug substance. In some embodiments, any of the ASOs disclosed herein are attached to a conjugated moiety that is an active drug substance. Examples of active drug substances include, but are not limited to, aspirin, warfarin, phenylbutazone, ibuprofen, suprofen, fenbufen, ketoprofen, (5)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Exemplary ASOs

As described above, the ASO disclosed herein may comprise a modified nucleotide such as Gutb, Nmln, 5prnl, G-clamp, or a combination thereof. Additionally or alternatively, the ASO disclosed herein may comprise at least one mesyl phosphoroamidate internucleoside linkage. Table 1 provides some exemplary ASO comprising either a modified nucleotide such as Gutb, Nmln, 5prnl, G-clamp, or a combination thereof, at least one mesyl phosphoroamidate internucleoside linkage (referred to in sequences as “yp”); or a combination thereof.

TABLE 1
Exemplary ASO Sequences

| SEQ ID | | Sequence |
|---|---|---|
| NO. | ASO # | (5′ to 3′) |
| 1 | 1 | lnGpslnApslnTpslnApslnAypApsAps(5oh)CpsGps(5m)Cps(5m)CpsGps(5m)Cpsln |
| | | ApslnGpslnApscp(5m)C |
| 2 | 2 | lnGpslnApslnTpslnApslnAypAypAps(5oh)CpsGps(5m)Cps(5m)CpsGps(5m)Cpsln |
| | | ApslnGpslnApscp(5m)C |
| 3 | 3 | lnGpslnApslnTpslnApslnAypAypAyp(5oh)CypGyp(5m)Cps(5m)CpsGps(5m)Cpsl |
| | | nApslnGpslnApscp(5m)C |
| 4 | 4 | lnGpslnApslnTpslnApslnAypApsAps(5oh)CpsGps(5m)Cps(5m)CpsGps(5m)Cpsln |
| | | ApslnGpslnApsln(5m)C |
| 5 | 5 | lnGpslnApslnTpslnApslnAypAypAps(5oh)CpsGps(5m)Cps(5m)CpsGps(5m)Cpsln |
| | | ApslnGpslnApsln(5m)C |
| 6 | 6 | lnGpslnApslnTpslnApslnAypAypAyp(5oh)CypGyp(5m)Cps(5m)CpsGps(5m)Cpsl |
| | | nApslnGpslnApsln(5m)C |
| 7 | 7 | lnGpslnApslnTps(Nmln)ApslnAypApsAps(5m)CpsGps(5m)Cps(5m)CpsGps(5m) |
| | | CpslnApslnGpslnApsln(5m)C |
| 8 | 8 | lnGpslnApslnTps(Nmln)ApslnAypAypAps(5m)CpsGps(5m)Cps(5m)CpsGps(5m) |
| | | CpslnApslnGpslnApsln(5m)C |
| 9 | 9 | lnGpslnApslnTps(Nmln)ApslnAypAypAyp(5m)CypGyp(5m)Cps(5m)CpsGps(5m) |
| | | CpslnApslnGpslnApsln(5m)C |
| 10 | 10 | lnGpslnApslnTps(Nmln)ApslnAypAypAyp(5m)CypGyp(5m)Cyp(5m)CypGyp(5m |
| | | )CpslnApslnGpslnApsln(5m)C |
| 11 | 11 | lnGpslnApslnTpslnApslnAypAypAyp(5oh)CypGyp(5m)Cyp(5m)CypGyp(5m)Cyp |
| | | lnApslnGpslnApscp(5m)C |
| 12 | 12 | lnGpslnApslnTpslnApslnAypApsAyp(5oh)CpsGyp(5m)Cps(5m)CypGps(5m)Cypl |
| | | nApslnGpslnApscp(5m)C |
| 13 | 13 | lnGyplnAyplnTyplnAyplnAypApsAps(5oh)CpsGps(5m)Cps(5m)CpsGps(5m)Cpsl |
| | | nAyplnGyplnAypcp(5m)C |
| 14 | 14 | lnGpslnApslnTpslnApslnApsApsAyp(5oh)CypGps(5m)Cps(5m)CpsGps(5m)Cpsln |
| | | ApslnGpslnApscp(5m)C |
| 15 | 15 | lnGpslnApslnTpslnApslnApsApsAps(5oh)CpsGps(5m)Cyp(5m)CypGps(5m)Cpsln |
| | | ApslnGpslnApscp(5m)C |
| 16 | 16 | lnGpslnApslnTpslnApslnApsApsAps(5oh)CpsGps(5m)Cps(5m)CypGyp(5m)Cpsln |
| | | ApslnGpslnApscp(5m)C |
| 17 | 17 | lnGpslnApslnTpslnApslnApsApsAps(5oh)CpsGps(5m)Cps(5m)CpsGyp(5m)Cypln |
| | | ApslnGpslnApscp(5m)C |
| 18 | 18 | lnGpslnApsScTypdTps(5m)Cps(8Am)ApsGps(5m)CpsGps(5m)Cps(5m)CpsGpsA |
| | | psln(5m)CpslnGpslnGpslnG |
| 19 | 19 | lnGpslnApsScTypdTyp(5m)Cps(8Am)ApsGps(5m)CpsGps(5m)Cps(5m)CpsGpsA |
| | | psln(5m)CpslnGpslnGpslnG |
| 20 | 20 | lnGpslnApsScTypdTyp(5m)Cyp(8Am)AypGyp(5m)CpsGps(5m)Cps(5m)CpsGpsA |
| | | psln(5m)CpslnGpslnGpslnG |
| 21 | 21 | lnGpslnApsScTypdTyp(5m)Cyp(8Am)AypGyp(5m)CypGyp(5m)Cyp(5m)CypGyp |
| | | Apsln(5m)CpslnGpslnGpslnG |
| 22 | 22 | lnGpslnApsScTypdTps(5m)Cyp(8Am)ApsGyp(5m)CpsGyp(5m)Cps(5m)CypGpsA |
| | | ypln(5m) CpslnGpslnGpslnG |
| 23 | 23 | GalNac4-ps2-p-mA- |
| | | lnGpslnApslnTpslnApslnAypAypAyp(5oh)CypGyp(5m)Cps(5m)CpsGps(5m)Cpsl |
| | | nApslnGpslnApscp(5m)C |
| 24 | 24 | GalNac4-ps2-p-mA- |
| | | lnGpslnApslnTpslnApslnAypAypAyp(5oh)CypGyp(5m)Cps(5m)CpsGps(5m)Cpsl |
| | | nApslnGpslnApsln(5m)C |
| 25 | 25 | lnGpslnApslnTpsTps(gcl)CpsApsGps(5m)CpsGps(5m)Cps(5m)CpsGpsApsln(5m) |
| | | CpslnGpslnGpslnG |
| 26 | 26 | lnGpslnApslnTpsTps(5m)CpsApsGps(gcl)CpsGps(5m)Cps(5m)CpsGpsApsln(5m) |
| | | CpslnGpslnGpslnG |
| 27 | 27 | lnGpslnApslnTpsTps(5m)CpsApsGps(5m)CpsGps(gcl)Cps(5m)CpsGpsApsln(5m) |
| | | CpslnGpslnGpslnG |
| 28 | 28 | lnGpslnApslnTpsTps(5m)CpsApsGps(5m)CpsGps(5m)Cps(gcl)CpsGpsApsln(5m) |
| | | CpslnGpslnGpslnG |
| 29 | 29 | lnGpslnApslnTpsTps(5m)CpsApsGps(5m)CpsGps(5m)Cps(5m)CpsGpsAps(gcl)C |
| | | pslnGpslnGpslnG |
| 30 | 30 | lnGpslnApslnTpsTps(5m)CpsApsGps(5m)CpsGps(5m)Cps(gcl)CpsGpsAps(gcl)C |
| | | pslnGpslnGpslnG |
| 31 | 31 | lnGpslnApslnTpslnApslnApsApsAps(gcl)CpsGps(5m)Cps(5m)CpsGps(5m)Cpsln |
| | | ApslnGpslnApsln(5m)C |
| 32 | 32 | lnGpslnApslnTpslnApslnApsApsAps(5m)CpsGps(gcl)Cps(5m)CpsGps(5m)Cpsln |
| | | ApslnGpslnApsln(5m)C |
| 33 | 33 | lnGpslnApslnTpslnApslnApsApsAps(5m)CpsGps(5m)Cps(gcl)CpsGps(5m)Cpsln |
| | | ApslnGpslnApsln(5m)C |
| 34 | 34 | lnGpslnApslnTpslnApslnApsApsAps(5m)CpsGps(5m)Cps(5m)CpsGps(gcl)Cpsln |
| | | ApslnGpslnApsln(5m)C |
| 35 | 35 | lnGpslnApslnTpslnApslnApsApsAps(5m)CpsGps(5m)Cps(5m)CpsGps(5m)Cpsln |
| | | ApslnGpslnAps(gcl)C |
| 36 | 36 | lnGpslnApslnTpslnApslnApsApsAps(5m)CpsGps(5m)Cps(5m)CpsGps(gcl)Cpsln |
| | | ApslnGpslnAps(gcl)C |
| 37 | 37 | lnGpslnApslnTpsTps(5prnl)CpsApsGps(5m)CpsGps(5m)Cps(5m)CpsGpsApsln(5 |
| | | m)CpslnGpslnGpslnG |
| 38 | 38 | lnGpslnApslnTpsTps(5m)CpsApsGps(5prnl)CpsGps(5m)Cps(5m)CpsGpsApsln(5 |
| | | m)CpslnGpslnGpslnG |
| 39 | 39 | lnGpslnApslnTpsTps(5m)CpsApsGps(5m)CpsGps(5prnl)Cps(5m)CpsGpsApsln(5 |
| | | m)CpslnGpslnGpslnG |
| 40 | 40 | lnGpslnApslnTpsTps(5m)CpsApsGps(5m)CpsGps(5m)Cps(5prnl)CpsGpsApsln(5 |
| | | m)CpslnGpslnGpslnG |
| 41 | 41 | lnGpslnApslnTpslnApslnApsApsAps(5prnl)CpsGps(5m)Cps(5m)CpsGps(5m)Cpsl |
| | | nApslnGpslnApsln(5m)C |
| 42 | 42 | lnGpslnApslnTpslnApslnApsApsAps(5m)CpsGps(5prnl)Cps(5m)CpsGps(5m)Cpsl |
| | | nApslnGpslnApsln(5m)C |
| 43 | 43 | lnGpslnApslnTpslnApslnApsApsAps(5m)CpsGps(5m)Cps(5prnl)CpsGps(5m)Cpsl |
| | | nApslnGpslnApsln(5m)C |
| 44 | 44 | lnGpslnApslnTpslnApslnApsApsAps(5m)CpsGps(5m)Cps(5m)CpsGps(5prnl)Cpsl |
| | | nApslnGpslnApsln(5m)C |
| 45 | 45 | lnGps(gutb)ApslnTpslnApslnApsApsAps(5m)CpsGps(5m)Cps(5m)CpsGps(5m)C |
| | | pslnApslnGpslnApsln(5m)C |
| 46 | 46 | lnGpslnAps(gutb)TpslnApslnApsApsAps(5m)CpsGps(5m)Cps(5m)CpsGps(5m)C |
| | | pslnApslnGpslnApsln(5m)C |
| 47 | 47 | lnGpslnApslnTps(gutb)ApslnApsApsAps(5m)CpsGps(5m)Cps(5m)CpsGps(5m)C |
| | | pslnApslnGpslnApsln(5m)C |
| 48 | 48 | lnGpslnApslnTpslnAps(gutb)ApsApsAps(5m)CpsGps(5m)Cps(5m)CpsGps(5m)C |
| | | pslnApslnGpslnApsln(5m)C |
| 49 | 49 | lnGpslnApslnTpslnApslnApsApsAps(5m)CpsGps(5m)Cps(5m)CpsGps(5m)Cps(gu |
| | | tb)ApslnGpslnApsln(5m)C |
| 50 | 50 | lnGpslnApslnTpslnApslnApsApsAps(5m)CpsGps(5m)Cps(5m)CpsGps(5m)Cpsln |
| | | ApslnGps(gutb)Apsln(5m)C |
| 51 | 51 | lnGpslnApslnTpslnApslnApsApsAps(5m)CpsGps(5m)Cps(5m)CpsGps(5m)Cpsln |
| | | ApslnGpslnAps(gutb)(5m)C |
| 52 | 52 | lnGps(gutb)ApslnTpsTps(5m)CpsApsGps(5m)CpsGps(5m)Cps(5m)CpsGpsApsln |
| | | (5m)CpslnGpslnGpslnG |
| 53 | 53 | lnGpslnAps(gutb)TpsTps(5m)CpsApsGps(5m)CpsGps(5m)Cps(5m)CpsGpsApsln |
| | | (5m)CpslnGpslnGpslnG |
| 54 | 54 | lnGpslnApslnTpsTps(5m)CpsApsGps(5m)CpsGps(5m)Cps(5m)CpsGpsAps(gutb) |
| | | (5m)CpslnGpslnGpslnG |
| 55 | 55 | GalNac4-ps2-p-mA- |
| | | lnGps(gutb)ApslnTpslnApslnApsApsAps(5m)CpsGps(5m)Cps(5m)CpsGps(5m)C |
| | | pslnApslnGpslnApsln(5m)C |
| 56 | 56 | GalNac4-ps2-p-mA- |
| | | lnGps(gutb)ApslnTpslnApslnApsApsAps(5oh)CpsGps(5m)Cps(5m)CpsGps(5m)C |
| | | pslnApslnGpslnApsln(5m)C |
| 57 | 57 | GalNac4-ps2-p-mA- |
| | | lnGps(gutb)ApslnTpslnApslnApsApsAps(5m)CpsGps(5m)Cps(5m)CpsGps(5m)C |
| | | pslnApslnGpslnApscp(5m)C |
| 58 | 58 | GalNac4-ps2-p-mA- |
| | | lnGps(gutb)ApslnTpslnApslnApsApsAps(5oh)CpsGps(5m)Cps(5m)CpsGps(5m)C |
| | | pslnApslnGpslnApscp(5m)C |
| 59 | 59 | GalNac4-ps2-p-mA- |
| | | lnGpslnApslnTpslnApslnApsApsAps(5m)CpsGps(5m)Cps(5m)CpsGps(5m)Cpsln |
| | | ApslnGpslnApsln(5m)C |
| 60 | 60 | GalNac4-ps2-p-mA- |
| | | lnGpslnAps(gutb)TpslnApslnApsApsAps(5m)CpsGps(5m)Cps(5m)CpsGps(5m)C |
| | | pslnApslnGpslnApsln(5m)C |
| 61 | 61 | GalNac4-ps2-p-mA- |
| | | lnGpslnAps(gutb)TpslnApslnApsApsAps(5oh)CpsGps(5m)Cps(5m)CpsGps(5m)C |
| | | pslnApslnGpslnApsln(5m)C |
| 62 | 62 | GalNac4-ps2-p-mA- |
| | | lnGpslnAps(gutb)TpslnApslnApsApsAps(5m)CpsGps(5m)Cps(5m)CpsGps(5m)C |
| | | pslnApslnGpslnApscp(5m)C |
| 63 | 63 | GalNac4-ps2-p-mA- |
| | | lnGpslnAps(gutb)TpslnApslnApsApsAps(5oh)CpsGps(5m)Cps(5m)CpsGps(5m)C |
| | | pslnApslnGpslnApscp(5m)C |
| 64 | 64 | lnGpslnApslnTpslnApslnApsApsAps(5oh)CpsGps(5m)Cps(5m)CpsGps(5m)Cpsln |
| | | ApslnGpslnApscp(5m)C |
| 65 | 65 | lnGpslnApslnTpslnApslnApsApsAps(5m)CpsGps(5m)Cps(5m)CpsGps(5m)Cpsln |
| | | ApslnGpslnApsln(5m)C |
| 66 | 66 | (Nmln)AGpslnApslnTpslnApslnApsApsAps(5m)CpsGps(5m)Cps(5m)CpsGps(5m) |
| | | CpslnApslnGpslnApsln(5m)C |
| 67 | 67 | lnGps(Nmln)ApslnTpslnApslnApsApsAps(5m)CpsGps(5m)Cps(5m)CpsGps(5m)C |
| | | pslnApslnGpslnApsln(5m)C |
| 68 | 68 | lnGpslnAps(Nmln)TpslnApslnApsApsAps(5m)CpsGps(5m)Cps(5m)CpsGps(5m) |
| | | CpslnApslnGpslnApsln(5m)C |
| 69 | 69 | lnGpslnApslnTps(Nmln)ApslnApsApsAps(5m)CpsGps(5m)Cps(5m)CpsGps(5m)C |
| | | pslnApslnGpslnApsln(5m)C |
| 70 | 70 | lnGpslnApslnTpslnAps(Nmln)ApsApsAps(5m)CpsGps(5m)Cps(5m)CpsGps(5m)C |
| | | pslnApslnGpslnApsln(5m)C |
| 71 | 71 | lnGpslnApslnTpslnApslnApsApsAps(5m)CpsGps(5m)Cps(5m)CpsGps(5m)Cps(N |
| | | mln)ApslnGpslnApsln(5m)C |
| 72 | 72 | lnGpslnApslnTpslnApslnApsApsAps(5m)CpsGps(5m)Cps(5m)CpsGps(5m)Cpsln |
| | | Aps(Nmln)GpslnApsln(5m)C |
| 73 | 73 | lnGpslnApslnTpslnApslnApsApsAps(5m)CpsGps(5m)Cps(5m)CpsGps(5m)Cpsln |
| | | ApslnGps(Nmln)Apsln(5m)C |
| 74 | 74 | lnGpslnApslnTpslnApslnApsApsAps(5m)CpsGps(5m)Cps(5m)CpsGps(5m)Cpsln |
| | | ApslnGpslnAps(Nmln)(5m)C |
| 75 | 75 | lnGpslnApscpTpsTps(5m)Cps(8nh)ApsGps(5m)CpsGps(5m)Cps(5m)CpsGpsApsl |
| | | n(5m)CpslnGpslnGpslnG |
| 76 | 76 | lnGpslnApslnTpsTps(5m)CpsApsGps(5m)CpsGps(5m)Cps(5m)CpsGpsApsln(5m) |
| | | CpslnGpslnGpslnG |
| 77 | 77 | (Nmln)GpslnApslnTpsTps(5m)CpsApsGps(5m)CpsGps(5m)Cps(5m)CpsGpsApsl |
| | | n(5m)CpslnGpslnGpslnG |
| 78 | 78 | lnGps(Nmln)ApslnTpsTps(5m)CpsApsGps(5m)CpsGps(5m)Cps(5m)CpsGpsApsln |
| | | (5m)CpslnGpslnGpslnG |
| 79 | 79 | lnGpslnAps(Nmln)TpsTps(5m)CpsApsGps(5m)CpsGps(5m)Cps(5m)CpsGpsApsl |
| | | n(5m)CpslnGpslnGpslnG |
| 80 | 80 | lnGpslnApslnTpsTps(5m)CpsApsGps(5m)CpsGps(5m)Cps(5m)CpsGpsApsln(5m) |
| | | CpslnGpslnGps(Nmln)G |
| 81 | 81 | lnGpslnApslnTpsTps(5m)CpsApsGps(5m)CpsGps(5m)Cps(5m)CpsGpsApsln(5m) |
| | | CpslnGps(Nmln)GpslnG |
| 82 | 82 | lnGpslnApslnTpsTps(5m)CpsApsGps(5m)CpsGps(5m)Cps(5m)CpsGpsApsln(5m) |
| | | Cps(Nmln)GpslnGpslnG |
| 83 | 83 | lnGpslnApslnTpsTps(5m)CpsApsGps(5m)CpsGps(5m)Cps(5m)CpsGpsAps(Nmln) |
| | | (5m)CpslnGpslnGpslnG |
| 84 | 84 | GalNAc4-ps2-p-mA- |
| | | lnGpslnApslnTps(Nmln)ApslnApsApsAps(5m)CpsGps(5m)Cps(5m)CpsGps(5m)C |
| | | pslnApslnGpslnApsln(5m)C |
| 85 | 85 | GalNAc4-ps2-p-mA- |
| | | lnGpslnApslnTps(Nmln)ApslnApsApsAps(5OH)CpsGps(5m)Cps(5m)CpsGps(5m) |
| | | CpslnApslnGpslnApsln(5m)C |
| 86 | 86 | GalNAc4-ps2-p-mA- |
| | | lnGpslnApslnTpslnApslnApsApsAps(5m)CpsGps(5m)Cps(5m)CpsGps(5m)Cpsln |
| | | ApslnGpslnApsln(5m)C |
| 87 | 87 | GalNAc4-ps2-p-mA- |
| | | lnGpslnApslnTps(Nmln)ApslnApsApsAps(5OH)CpsGps(5m)Cps(5m)CpsGps(5m) |
| | | CpslnApslnGpslnApscp(5m)C-3′ |
| 88 | 88 | lnGyplnAyplnTyplnAyplnAypApsAps(5oh)CpsGps(5m)Cps(5m)CpsGps(5m)Cypl |
| | | nAyplnGyplnAypcp(5m)C |

In Table 1, the bold nucleosides contain one of the following modifications:

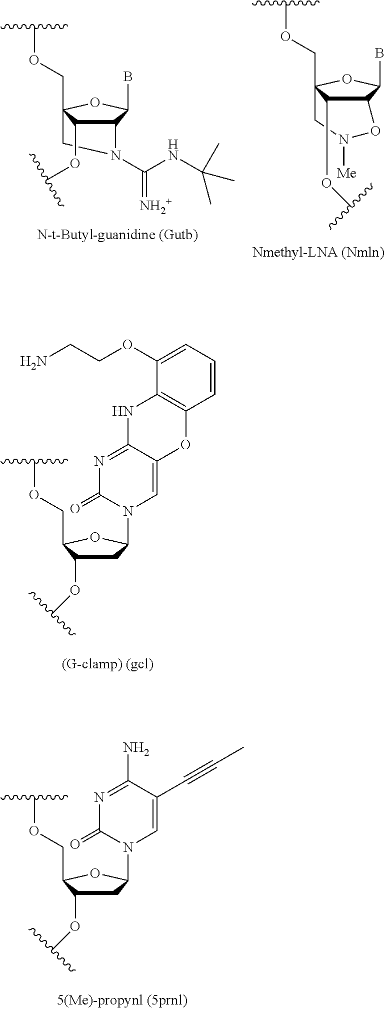

Pharmaceutical Compositions

The present disclosure also encompasses pharmaceutical compositions comprising ASOs of the present disclosure. One embodiment is a pharmaceutical composition comprising one or more ASO of the present disclosure, and a pharmaceutically acceptable diluent or carrier.

In some embodiments, the pharmaceutical composition containing the ASO of the present disclosure is formulated for systemic administration via parenteral delivery. Parenteral administration includes intravenous, intra-arterial, subcutaneous, intraperitoneal or intramuscular injection or infusion; also subdermal administration, e.g., via an implanted device. In a preferred embodiment, the pharmaceutical composition containing the ASO of the present disclosure is formulated for subcutaneous (SC) or intravenous (IV) delivery. Formulations for parenteral administration may include sterile aqueous solutions, which may also contain buffers, diluents and other pharmaceutically acceptable additives as understood by the skilled artisan. For intravenous use, the total concentration of solutes may be controlled to render the preparation isotonic.

The pharmaceutical compositions containing the ASO of the present disclosure are useful for treating a disease or disorder, e.g., associated with the expression or activity of an HBV gene.

In some embodiments, the pharmaceutical composition comprises a first ASO of the present disclosure that is complementary or hybridizes to a viral target RNA sequence in a first X region of HBV, and a second ASO of the present disclosure that is complementary or hybridizes to a viral target RNA sequence in a second X region or an S region of HBV, and a pharmaceutically acceptable diluent or carrier. When the pharmaceutical composition comprises two or more ASOs, the ASOs may be present in varying amounts. For example, in some embodiments, the weight ratio of first ASO to second ASO is 1:4 to 4:1, e.g., 1:4, 1:3, 1:2, 1:1, 2:1, 3:1, or 4:1. In some embodiments, the molar ratio of first ASO to second ASO is 1:4 to 4:1, e.g., 1:4, 1:3, 1:2, 1:1, 2:1, 3:1, or 4:1.

Treatments

The siNA molecules and compositions described herein may be administered to a subject to treat a disease. Further disclosed herein are uses of any of the siNA molecules or compositions disclosed herein in the manufacture of a medicament for treating a disease. In particular, the present disclosure provides ASO for the treatment of various disease, such as infectious diseases, including but not limited viral diseases and liver diseases.

One aspect of the present disclosure includes methods for treating a subject diagnosed as having, suspected as having, or at risk of having an HBV infection and/or an HBV-associated disorder. In therapeutic applications, compositions comprising at least one disclosed ASO are administered to a subject suspected of, or already suffering from such a disease (such as, e.g., persistence of HBV cccDNA, presence of an HBV antigen (e.g., HBsAg and/or HBeAg) in the serum and/or liver of the subject, or elevated HBV viral load levels), in an amount sufficient to cure, or at least partially arrest, the disease, including its complications and intermediate pathological phenotypes in development of the disease.

Subjects suffering from an HBV infection and/or an HBV-associated disorder can be identified by any or a combination of diagnostic or prognostic assays known in the art. For example, typical symptoms of HBV infection and/or an HBV-associated disorder include, but are not limited to the presence of liver HBV cccDNA, the presence of serum and/or liver HBV antigen (e.g., HBsAg and/or HBeAg), elevated ALT, elevated AST, the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), high levels of alanine aminotransferase (ALT) and aspartate aminotransferase (AST) (within a range of 1000-2000 IU/mL), ALT levels higher than AST levels, elevated gamma-glutamyl transpeptidase (GGT) and/or alkaline phosphatase (ALP) levels, decreased albumin levels, elevated serum iron levels, leukopenia (i.e., granulocytopenia), lymphocytosis, increased erythrocyte sedimentation rate (ESR), shortened red blood cell survival, hemolysis, thrombocytopenia, a prolongation of the international normalized ratio (INR), the presence of serum HBV DNA, elevation of the aminotransferases (<5 times the ULN), increased bilirubin levels, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, elevated levels of rheumatoid factor (RF), hyperbilirubinemia, low platelet and white blood cell counts, AST levels higher than ALT levels, lobular inflammation accompanied by degenerative and regenerative hepatocellular changes, and predominantly centrilobular necrosis.

In some embodiments, subjects treated with a disclosed ASO will show amelioration or elimination of one or more of the following conditions or symptoms: presence of liver HBV cccDNA, the presence of serum and/or liver HBV antigen (e.g., HBsAg and/or HBeAg), the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), ALT levels higher than AST levels, leukopenia (i.e., granulocytopenia), decreased albumin levels, elevated serum iron levels, lymphocytosis, increased erythrocyte sedimentation rate (ESR), shortened red blood cell survival, hemolysis, thrombocytopenia, a prolongation of the international normalized ratio (INR), the presence of serum HBV DNA, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, hyperbilirubinemia, low platelet and white blood cell counts, AST levels higher than ALT levels, lobular inflammation accompanied by degenerative and regenerative hepatocellular changes, and predominantly centrilobular necrosis.

The present disclosure provides a method for treating a subject diagnosed as having, or suspected as having an HBV infection and/or an HBV-associated disorder comprising administering to the subject an effective amount of an ASO composition of the present disclosure. In some embodiments, the method comprises administering to the subject a first ASO of the present disclosure and a second ASO of the present disclosure, wherein the first ASO is complementary or hybridizes to a viral target RNA sequence in a first X region of HBV, and the second ASO is complementary or hybridizes to a viral target RNA sequence in a second X region or an S region of HBV. In some embodiments, the second ASO is complementary or hybridizes to the viral target RNA sequence in the second X region of HBV. In other embodiments, the second ASO is complementary or hybridizes to the viral target RNA sequence in the S region of HBV.

In some embodiments of the disclosed methods and uses, the disease is a respiratory disease. In some embodiments, the respiratory disease is a viral infection. In some embodiments, the respiratory disease is viral pneumonia. In some embodiments, the respiratory disease is an acute respiratory infection. In some embodiments, the respiratory disease is a cold. In some embodiments, the respiratory disease is severe acute respiratory syndrome (SARS). In some embodiments, the respiratory disease is Middle East respiratory syndrome (MERS). In some embodiments, the disease is coronavirus disease 2019 (e.g., COVID-19). In some embodiments, the respiratory disease can include one or more symptoms selected from coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, haemoptysis, conjunctival congestion, sputum production, chest tightness, and palpitations. In some embodiments, the respiratory disease can include complications selected from sinusitis, otitis media, pneumonia, acute respiratory distress syndrome, disseminated intravascular coagulation, pericarditis, and kidney failure. In some embodiments, the respiratory disease is idiopathic.

In some embodiments, the present disclosure provides methods of treating or preventing a coronavirus infection, comprising administering to a subject in need thereof a therapeutically effective amount of one or more of the ASOs or a pharmaceutical composition as disclosed herein. In some embodiments, the coronavirus infection is selected from the group consisting of Middle East Respiratory Syndrome (MERS), Severe Acute Respiratory Syndrome (SARS), and COVID-19. In some embodiments, the subject has been treated with one or more additional coronavirus treatment agents. In some embodiments, the subject is concurrently treated with one or more additional coronavirus treatment agents.

In some embodiments, the disease is a liver disease. In some embodiments, the liver disease is nonalcoholic fatty liver disease (NAFLD). In some embodiments, the NAFLD is nonalcoholic steatohepatitis (NASH). In some embodiments, the liver disease is hepatocellular carcinoma (HCC).

The ASOs of the present disclosure may be used to treat a disease in a subject in need thereof. In some embodiments, a method of treating a disease in a subject in need thereof comprises administering to the subject any of the ASOs disclosed herein. In some embodiments, a method of treating a disease in a subject in need thereof comprises administering to the subject any of the compositions disclosed herein.

Administration of the ASO may be conducted by methods known in the art. In some embodiments, the ASO is administered by subcutaneous (SC) or intravenous (IV) delivery. The preparations (e.g., ASOs or compositions) of the present disclosure may be given orally, parenterally, topically, or rectally. They are of course given in forms suitable for each administration route. For example, they are administered in tablets or capsule form, administration by injection, infusion or inhalation; topical by lotion or ointment; and rectal by suppositories. In some embodiments, subcutaneous administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally, and sublingually.

Regardless of the route of administration selected, the compounds (e.g., ASOs) of the present disclosure, which may be used in a suitable hydrated form, and/or the pharmaceutical compositions of the present disclosure, are formulated into pharmaceutically-acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound (e.g., ASO) of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds (e.g., ASOs) of the disclosure employed in the pharmaceutical composition at levels lower than that required in order to achieve the desired therapeutic effect and gradually increase the dosage until the desired effect is achieved.

In general, a suitable daily dose of a compound (e.g., ASO) of the disclosure is the amount of the compound that is the lowest dose effective to produce a therapeutic effect. Such an effective dose generally depends upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In some embodiments, the compound is administered at about 1 mg/kg to about 40 mg/kg, about 1 mg/kg to about 30 mg/kg, about 1 mg/kg to about 20 mg/kg, about 1 mg/kg to about 15 mg/kg, or 1 mg/kg to about 10 mg/kg. In some embodiments, the compound is administered at a dose equal to or greater than 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.7, 0.75, 0.8, 0.85, 0.9, 0.95, or 1 mg/kg. In some embodiments, the compound is administered at a dose equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg. In some embodiments, the compound is administered at a dose equal to or less than 200, 190, 180, 170, 160, 150, 140, 130, 120, 110, 100, 95, 90, 85, 80, 75, 70, 65, 60, 55, 50, 45, 40, 35, 30, 25, 20, or 15 mg/kg. In some embodiments, the total daily dose of the compound is equal to or greater than 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 100 mg.

If desired, the effective daily dose of the active compound (e.g., ASO) may be administered as two, three, four, five, six, seven, eight, nine, ten or more doses or sub-doses administered separately at appropriate intervals throughout the day, optionally, in unit dosage forms. In some embodiments, the compound is administered at least 1, 2, 3, 4, 5, 6, 7, 8, 9, 10, 11, 12, 13, 14, or 15 times. Preferred dosing is one administration per day. In some embodiments, the compound is administered at least 1, 2, 3, 4, 5, 6, 7, 8, 9, 10, 11, 12, 13, 14, 15, 16, 17, 18, 19, 20, or 21 times a week. In some embodiments, the compound is administered at least 1, 2, 3, 4, 5, 6, 7, 8, 9, 10, 11, 12, 13, 14, 15, 16, 17, 18, 19, 20, or 21 times a month. In some embodiments, the compound is administered once every 1, 2, 3, 4, 5, 6, 7, 8, 9, 10, 11, 12, 13, 14, 15, 16, 17, 18, 19, 20, or 21 days. In some embodiments, the compound is administered every 3 days. In some embodiments, the compound is administered once every 1, 2, 3, 4, 5, 6, 7, 8, 9, 10, 11, 12, 13, 14, or 15 weeks. In some embodiments, the compound is administered every month. In some embodiments, the compound is administered once every 1, 2, 3, 4, 5, 6, 7, 8, 9, 10, 11, 12, 13, 14, or 15 months. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 weeks. In some embodiments, the compound is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 times over a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 months. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least twice a week for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every two weeks for a period of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weeks. In some embodiments, the compound is administered at least once every four weeks for a period of at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 months.

The subject of the described methods may be a mammal, and it includes humans and non-human mammals. In some embodiments, the subject is a human, such as an adult human.

Some embodiments include a method for treating an HBV virus in a subject infected with the virus comprising administering a therapeutically effective amount of one or more ASO of the present disclosure or a composition of the present disclosure to the subject in need thereof thereby reducing the viral load of the virus in the subject and/or reducing a level of a virus antigen in the subject. The ASO may be complementary or hybridize to a portion of the target RNA in the virus, e.g., a second X region and/or an S region of HBV.

In some embodiments, a modified oligonucleotide as described herein can be used in combination with one or more additional agent(s) for treating and/or inhibiting replication HBV and/or HDV. When the compounds (e.g., ASOs) described herein are co-administered with an additional agent, the effective amount may be less than when the compound is used alone. Additional agents include, but are not limited to, an interferon, nucleoside/nucleotide analogs, a capsid assembly modulator (CAM), siRNA, other ASOs, Nucleic Acid Polymers or S-Antigen Transport-inhibiting Oligonucleotide Polymers (NAPs or STOPS), an entry inhibitor and/or a small molecule immunomodulator. Examples of additional agents include ALG-010133, ALG-000184, recombinant interferon alpha 2b, IFN-α, PEG-IFN-α-2a, lamivudine, telbivudine, adefovir dipivoxil, clevudine, entecavir, tenofovir alafenamide, tenofovir disoproxil, NVR3-778, BAY41-4109, JNJ-632, JNJ-3989 (ARO-HBV), RG6004, GSK3228836, REP-2139, REP-2165, AB-729, VIR-2218, DCR-HBVS, JNJ-6379, GLS4, ABI-HO731, JNJ-440, NZ-4, RG7907, EDP-514, AB-423, AB-506, ABI-H03733 and ABI-H2158. In some embodiments, any of the ASOs disclosed herein are co-administered with one of STOPS. Exemplary STOPS are described in International Publication No. WO2020/097342 and U.S. Publication No. 2020/0147124, both of which are incorporated by reference in their entirety. In some embodiments, the STOP is ALG-010133. In some embodiments, any of the ASOs disclosed herein are co-administered with tenofovir. In some embodiments, any of the ASOs disclosed herein are co-administered with a CAM. Exemplary CAMs are described in Berke et al., Antimicrob Agents Chemother, 2017, 61(8):e00560-17, Klumpp, et al., Gastroenterology, 2018, 154(3):652-662.e8, International Application Nos. PCT/US2020/017974, PCT/US2020/026116, and PCT/US2020/028349 and U.S. application Ser. Nos. 16/789,298, 16/837,515, and 16/849,851, each which is incorporated by reference in its entirety. In some embodiments, the CAM is ALG-000184, ALG-001075, ALG-001024, JNJ-632, BAY41-4109, or NVR3-778. In some embodiments, the ASO and the additional agent are administered simultaneously. In some embodiments, the ASO and the additional agent are administered sequentially. In some embodiments, the ASO is administered prior to administering the additional agent. In some embodiments, the ASO is administered after administering the additional agent.

### Definitions

As used herein, the terms “patient” and “subject” refer to organisms to be treated by the methods of the present disclosure. Such organisms are preferably mammals (e.g., marines, simians, equines, bovines, porcinis, canines, felines, and the like), and more preferably humans.

As used herein, the term “effective amount” refers to the amount of a compound (e.g., a ASO of the present disclosure) sufficient to effect beneficial or desired results. An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the terms “alleviate” and “alleviating” refer to reducing the severity of the condition, such as reducing the severity by, for example, at least 10%, 20%, 30%, 40%, 50%, 60%, 70%, 80%, 90%, or 95%.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA [1975].

The term “about” as used herein when referring to a measurable value (e.g., weight, time, and dose) is meant to encompass the value recited and a range of +/−10%. For example, “about 10” should be understood as both “10” and “9 to 11”.

As used herein, the term “nucleobase” or “base” refers to a nitrogen-containing biological compound that forms a nucleoside. Examples of nucleobases include, but are not limited to, thymine, uracil, adenine, cytosine, guanine, and an analogue or derivative thereof.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

This disclosure is not limited to particular embodiments described, as such may vary. It is also to be understood that the terminology used herein is for the purpose of describing particular embodiments only, and is not intended to be limiting, since the scope of the present invention will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

### EXAMPLES

The following examples illustrate certain embodiments of the present disclosure to aid the skilled person in practicing the disclosure. Accordingly, the examples are in no way considered to limit the scope of the disclosure.

### Example 1: ASO Synthesis

Gapmer ASO Sequences: The DNA, 2′-O-Me, and LNA phosphoramidite monomers were procured from commercially available sources (Hongene Biotech USA Inc.). All the monomers were dried in vacuum desiccator with desiccants (P₂O₅, RT 24h). Universal solid supports (CPG) attached were obtained from ChemGenes corporation. The chemicals and solvents for synthesis workflow were purchased from VWR/Sigma commercially available sources and used without any purification or treatment. Solvent (acetonitrile) and solutions (amidite and activator) were stored over molecular sieves during synthesis.

The control and target oligonucleotide sequences were synthesized on an Expedite 8909 synthesizer using the standard cycle written by the manufacturer with modifications to a few wait steps and modified coupling steps. The solid support was controlled pore glass and the monomers contained standard protecting groups. Each chimeric oligonucleotide was individually synthesized using commercially available 5′-O-(4,4′-dimethoxytrityl)-3′-O-(2-cyanoethyl-N, N-diisopropyl) DNA, 2′-OMe, and or LNA phosphoramidite monomers of 6-N-benzoyladenosine (A^{Bz}), 5 methyl 4-N-benzoyltidine (C^{Bz}), 2-N-isobutyrylguanosine (G^{iBu}), and Uridine (U) or Thymidine (T), according to standard solid phase Phosphoramidite synthesis protocols. The 2′-O-Me-2,6-diaminopurine phosphoramidite was purchased from Glen Research. The phosphoramidites were prepared as 0.1 M solutions in anhydrous acetonitrile. 5-Ethylthiotetrazole was used as activator, 3% dichloroacetic acid in dichloromethane was used to detritylate, acetic anhydride in THE and 16% N-methylimidazole in THE were used to cap, and DDTT ((dimethylamino-methylidene) amino)-3H-1,2,4-dithiazaoline-3-thione was used as the sulfur-transfer agent for the synthesis of oligoribonucleotide phosphorothioates. An extended coupling of 0.1M solution of phosphoramidite in CH₃CN in the presence of 5-(ethylthio)-1H-tetrazole activator to a solid bound oligonucleotide followed by extended capping, oxidation and deprotection afforded modified oligonucleotides. The stepwise coupling efficiency of all modified phosphoramidites was more than 98.5%.

Deprotection and cleavage from the solid support was achieved with mixture of ammonia methylamine (1:1, AMA) for 15 min at 65° C., when the universal linker was used, the deprotection was left for 90 min at 65° C. or solid supports were heated with aqueous ammonia (28%) solution at 55° C. for 8 h to deprotect the base labile protecting groups.

After filtering to remove the solid support, the deprotection solution was removed under vacuum in a GeneVac centrifugal evaporator.

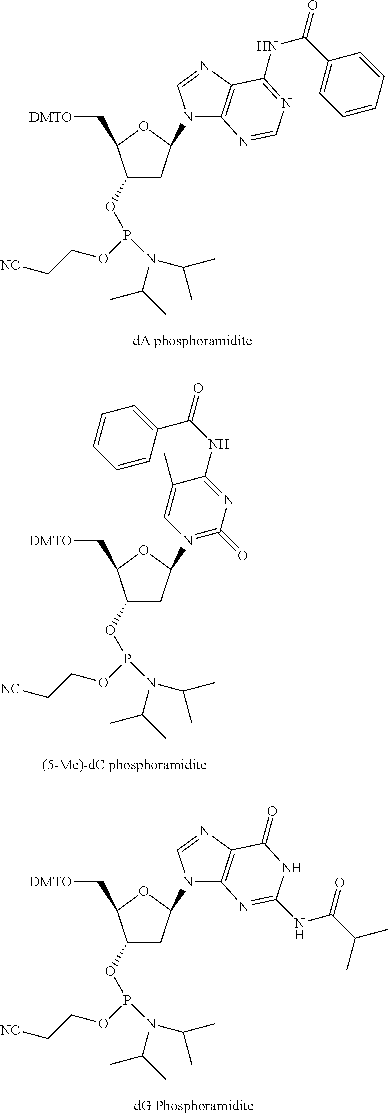

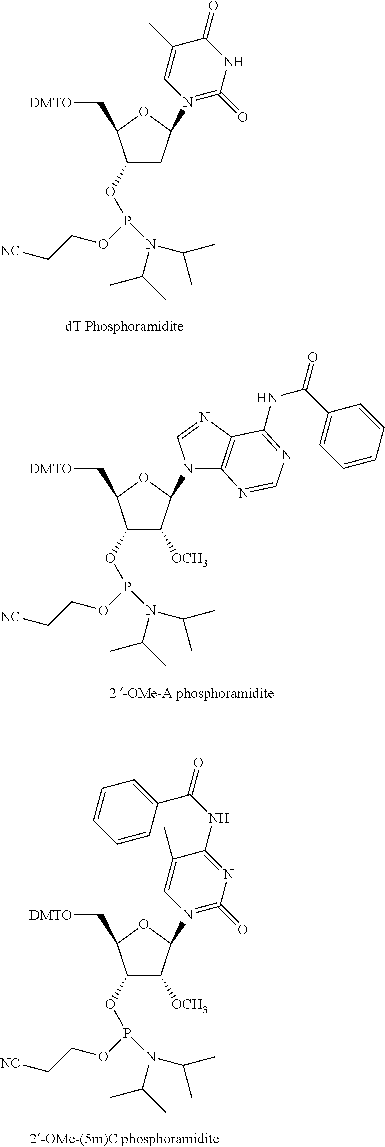

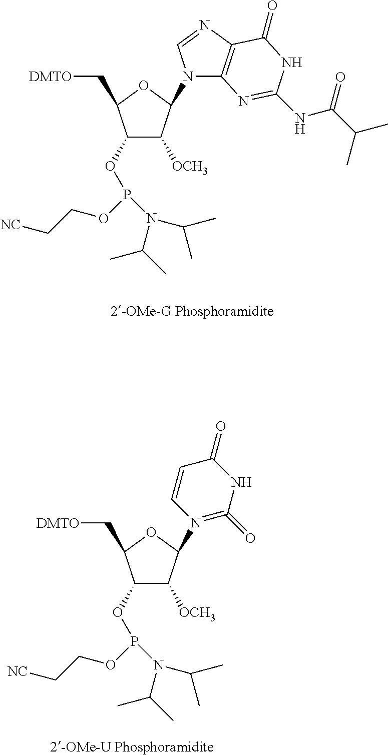

### The Locked Nucleic Acid (LNA) Phosphoramidite

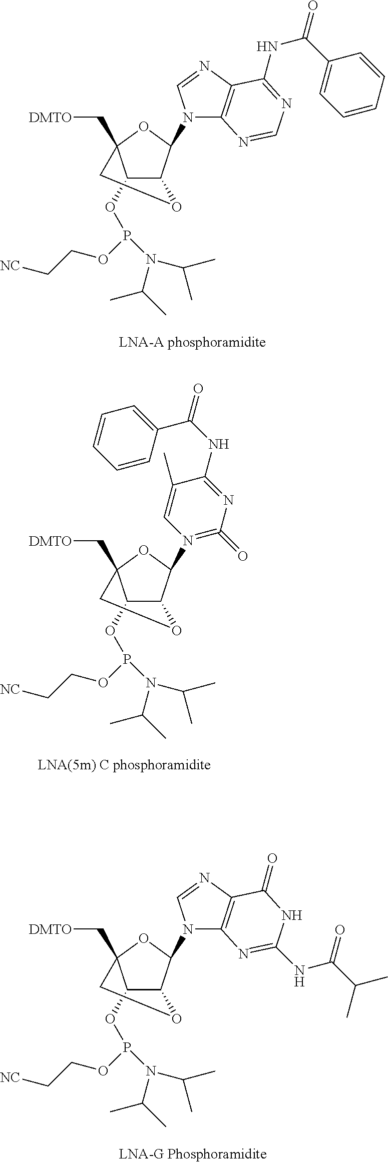

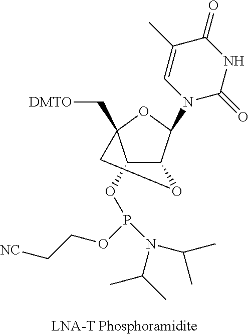

### Example 2: Modified Gapmer Sequences

The AmNA (N-Me)-T, AmNA (N-Me)-4-N-benzoyl (5m) cytidine ((5m) C^{Bz}), AmNA (N-Me)-4-N-benzoylcytidine (A^{Bz}), and AmNA (N-Me)-2-N-pac (G^{pac}), were purchased from Luxna Biotech, whereas scp-BNA-T, scp-BNA-6-N-benzoyladenosine (A^{Bz}), scp-BNA-4-N-benzoyl-5 methyl cytidine ((5m) C^{Bz}), scp-BNA-2-N-isobutrylguanosine (G^{iBu}) phosphoramidite monomers synthesized by following the procedure described in references (Takao Yamaguchi, Masahiko Horiba and Satoshi Obika; Chem. Commun., 2015, 51, 9737-9740; Masahiko Horiba, Takao Yamaguchi, and Satoshi Obika; Journal of Organic Chemistry, 2016, 81, 11000-11008). All the monomers were dried in a vacuum desiccator with desiccants (KOH and P₂O₅, at room temperature for 24 hours). In the case of AmNA(N-Me)-PS-DNA-PS and scp-BNA-PS-DNA-PS, modifications the synthesis was carried out on a 1 μM scale in a 3′ to 5′ direction with the phosphoramidite monomers diluted to a concentration of 0.12 M in anhydrous CH₃CN in the presence of 0.3 M 5-(benzylthio)-1H-tetrazole activator (coupling time 16 min) to a solid bound oligonucleotide followed by modified capping, oxidation and deprotection afforded modified oligonucleotides. The stepwise coupling efficiency of all modified phosphoramidites was more than 97%. The DDTT (dimethylamino-methylidene) amino)-3H-1,2,4-dithiazaoline-3-thione was used as the sulfur-transfer agent for the synthesis of oligoribonucleotide phosphorothioates. Oligonucleotide-bearing solid supports were washed with 20% DEA solution in acetonitrile for 15 min then column was washed thoroughly with MeCN. The support was heated at 65° C. with diisopropyl amine: water: methanol (1:1:2) for 8 h in heat block to cleavage from support and deprotect the base labile protecting groups.

### AmNA (N-Me) Monomers

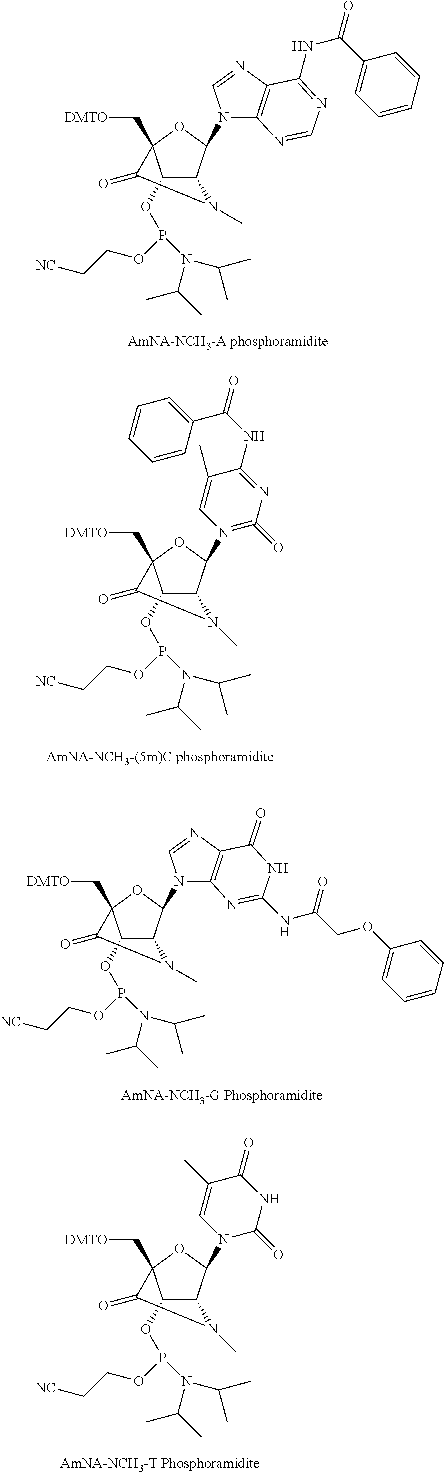

scp-BNA-A Monomers

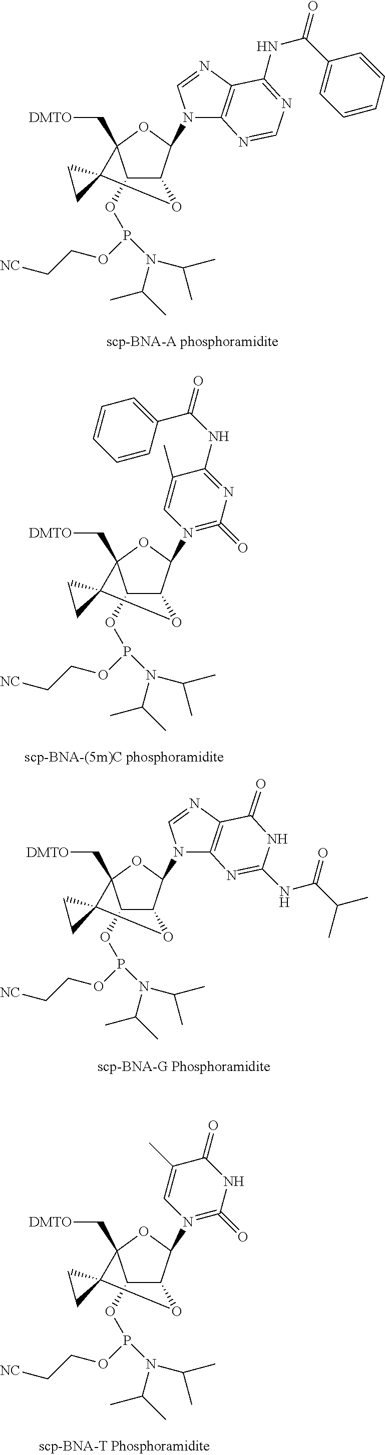

5′ and 3′-GalNAc conjugated oligonucleotides were synthesized with various length GalNAc moieties, e.g., as described below. The GalNAc3, GalNAc4, GalNAc5 and GalNAc6 were conjugated to oligonucleotides during synthesis with 1 2, or 3 moieties in the same manner as described below. Further GalNAc moieties, such as GalNAc-1 and GalNAc-2, which are described previously herein, are also used to form 5′ and 3′-GalNAc using post synthesis conjugation.

### GalNAc Phosphoramidites

| GalNAc building blocks | After Attachment to Oligos (Nomenclature) |
|---|---|
| GalNAc-3 phosphoramidite 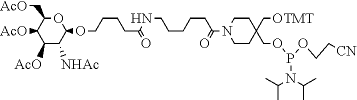 | 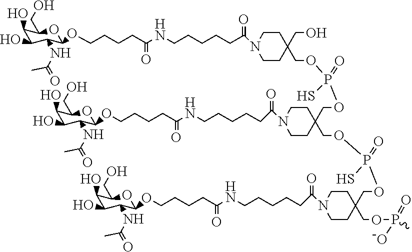 |
| | (GalNAc3-(PS)2-p) |
| GalNAc-4 phosphoramidite 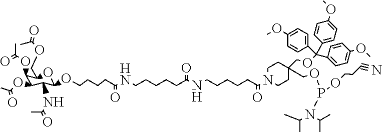 | 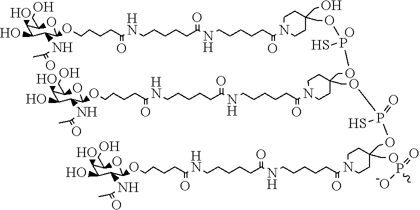 |
| | (GalNAc4-(PS)2-p) |
| GalNAc-5 phosphoramidite 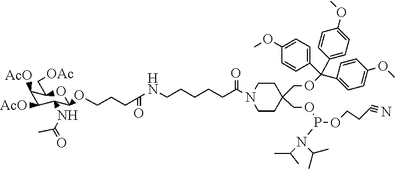 | 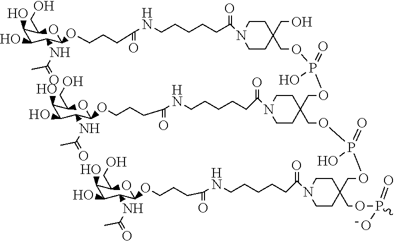 |
| | (GalNAc5-(PS)2-p) |
| GalNAc-6 phosphoramidite | 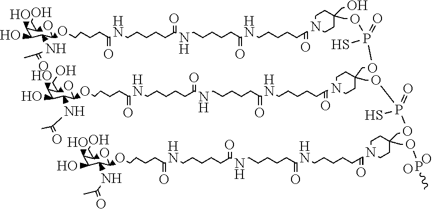 (GalNAc6-(PS)2-p) |

Quantitation of Crude Oligomer or Raw Analysis

Samples were dissolved in deionized water (1.0 mL) and quantitated as follows: Blanking was first performed with water alone on Nanodrop UV spectrophotometer. Nano Drop instruments can measure a wide concentration range of nucleic acids through use of multiple path lengths. The most accurate quantification results can be achieved by measuring diluted oligonucleotides with an absorbance at 260 nm. The crude material is stored at −20° C.

Crude HPLC/LC-MS Analysis

The 0.1 OD of the crude samples were used for crude MS analysis. After confirming the crude LC-MS data, then the purification step was performed.

HPLC Purification

The Phosphodiester (PO), Phosphorothioate (PS) and chimeric modified oligonucleotides were purified by anion-exchange HPLC. The buffers were 20 mM sodium phosphate in 10% CH₃CN, pH 8.5 (buffer A) and 20 mM sodium phosphate in 10% CH₃CN, 1.8 M NaBr, pH 8.5 (buffer B). Fractions containing full-length oligonucleotides were pooled, desalted and lyophilized.

The lipid conjugated oligonucleotides were purified by an in-house packed RPC-Source15 reverse-phase column. The buffers were 20 mM sodium acetate in 10% CH₃CN, (buffer A) and CH₃CN (buffer B). Fractions containing full-length oligonucleotides were pooled, desalted and lyophilized.

Desalting of Purified Oligomer

The purified dry oligomer was then desalted using Sephadex G-25 M (Amersham Biosciences). The cartridge was conditioned with 10 mL of deionized water thrice. The purified oligonucleotide dissolved thoroughly in 2.5 mL deionized water was applied to the cartridge with very slow drop wise elution. The salt free oligomer was eluted with 3.5 ml deionized water directly into a screw cap vial.

Final HPLC and Electrospray LC/MS Analysis

Approximately 0.10 OD of oligomer is dissolved in water and then pipetted in special vials for IEX-HPLC and LC/MS analysis. Analytical HPLC and ES LC-MS established the integrity of the chimeric oligonucleotides.

Post-Synthesis Conjugation of GalNAc esters to Oligonucleotides

5′-C6-Amino Precursor Synthesis

The sequences were synthesized at 10 μmol scale using universal support (Loading 65 μmol/g). At the 5′-terminal to introduce C6-NH₂ linker the 6-(4-monomethoxytritylamino)hexyl-(2-cyanoethyl)-(N, N-diisopropyl)-phosphoramidite in 0.1 M Acetonitrile was used with coupling time 10 min. The Oligonucleotide-bearing solid supports were heated at room temperature with aqueous ammonia/Methylamine (1:1) solution for 3 h in shaker to cleavage from support and deprotect the base labile protecting groups. After IEX purification and desalting the C6-NH₂ modified ASO's was used to perform post synthesis conjugation.

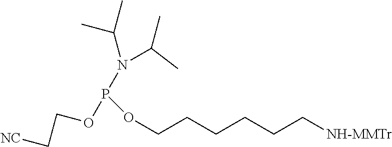

### 5′-Amino-Modifier C6

### 6-(4-Monomethoxytritylamino)hexyl-(2-cyanoethyl)-(N,N-diisopropyl)-phosphoramidite

### GalNAc Ester for Conjugation

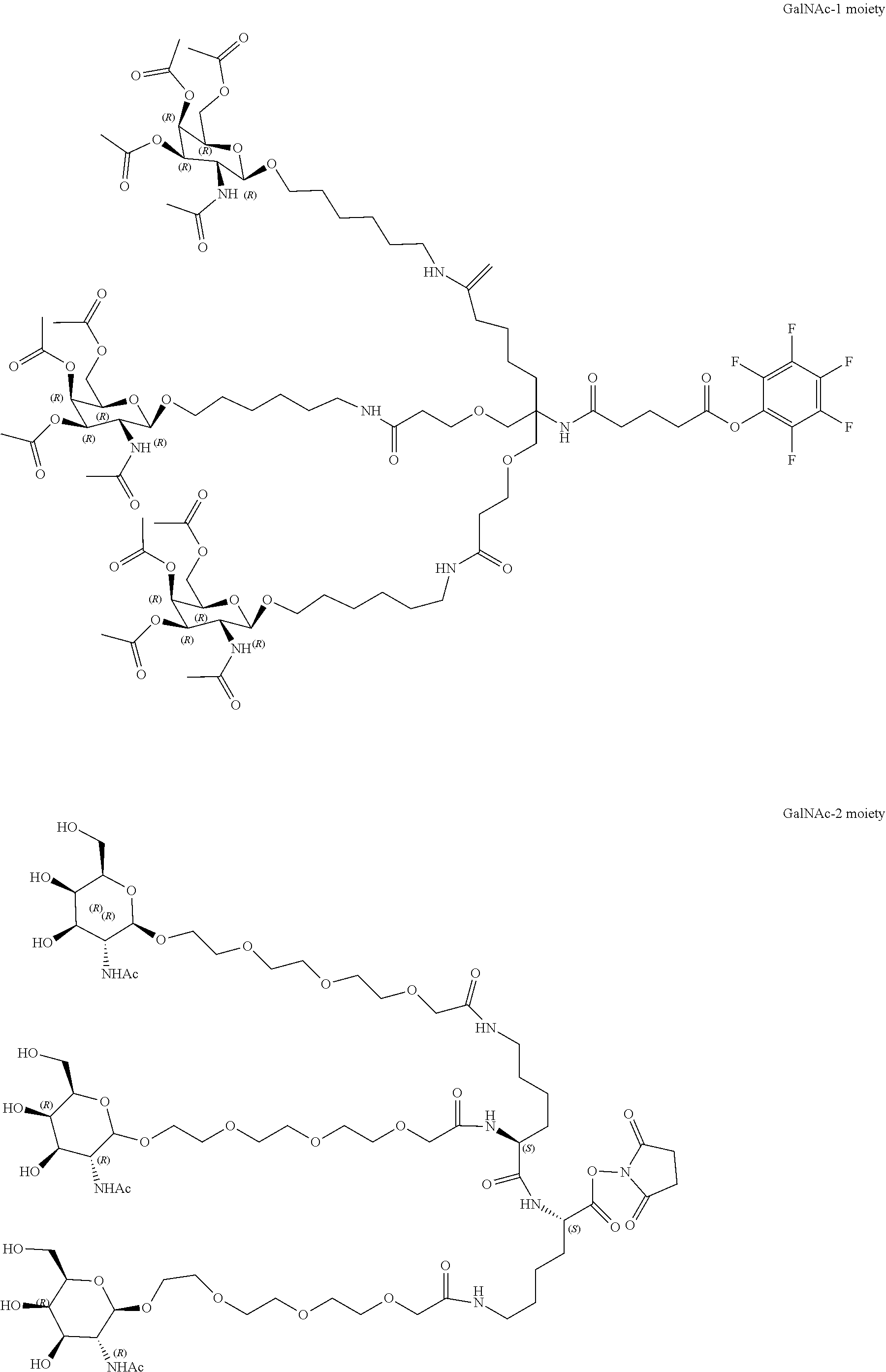

Post Synthesis Conjugation of 5′-GalNAc Synthesis

The 5′-C6-NH₂ modified sequences were dissolved in 0.2 M Sodium bicarbonate buffer, pH 8.5 (0.015 mM) and 5-7 mol equivalent of GalNAc ester dissolved in DMSO was added. The reaction mixture was stirred at room temperature for 4 h. The sample was analyzed to confirm if any unreacted amino modified ASO's is present. To this aqueous ammonia (28 wt. %) was added (5× reaction volume) and stirred at room temperature for 2-3 h. Reaction mixture concentrated under reduced pressure and residue dissolved in water and purified by HPLC on a strong anion exchange column.

### Example 3: HBsAg Release Assay In Vitro Analysis

HepG2.2.15 cells (a stable cell line with four integrated HBV genomes) were maintained in DMEM medium with 10% fetal bovine serum (FBS) and 1% penicillin/streptomycin, 1% Glutamine, 1% non-essential amino acids, 1% Sodium Pyruvate and 250 μg/ml G418. Cells were maintained at 37° C. in a 5% CO₂ atmosphere. For HBsAg release assay, assay medium was made: DMEM with 5% FBS, 1% penicillin/streptomycin, 1% Glutamine and 1% DMSO. The day before assay, trypsinize HepG2.2.15 cells were washed with Assay Medium once, spun at 250 g×5 min, resuspended with Assay Medium, and seed cells at 50,000/well in assay medium in collagen coated 96 well plates. On the next day, ASOs were diluted with Opti-MEM, 9-pt, 3-fold dilution and Lipofectamine RNAiMAX (Invitrogen) was diluted according manufacturer's manual. The ASO dilution and RNAiMAX dilution was mixed, left at room temperature for 5 minutes and 15 μl was added to each well of 96 well plate. The plates were left at 37° C., 5% CO₂ in an incubator for 5 days. After incubation, the supernatant was harvested and measured for HBsAg with ELISA kit (Diasino). The cell viability was measured with CellTiter-Glo (Promega). The EC₅₀, the concentration of the drug required for reducing HBsAg secretion by 50% in relation to the untreated cell control was calculated using the Prism Graphpad. The CC₅₀, the concentration of the drug required for reducing cell viability by 50% in relation to the untreated cell control was calculated with the same software.

The resulting EC₅₀ and CC₅₀ for the compounds in Table 1 are presented in the following Table 2. The EC₅₀ and CC₅₀ values are as follows: A: <1 nM, B: 1-10 nM, C: 10-100 nM, D: >100 nM.

TABLE 2
HBsAg Release Assay

| | HepG2.2.15 HBsAg Release | HepG2.2.15 Cell Viability |
|---|---|---|
| ASO # | Assay EC₅₀ (nM) | Assay CC₅₀ (nM) |
| 1 | B | D |
| 2 | C | D |
| 3 | B | D |
| 4 | C | D |
| 5 | B | D |
| 6 | B | D |
| 7 | B | D |
| 8 | B | D |
| 9 | B | D |
| 10 | B | D |
| 11 | D | D |
| 12 | D | D |
| 13 | | |
| 14 | | |
| 15 | | |
| 16 | | |
| 17 | | |
| 18 | | |
| 19 | | |
| 20 | | |
| 21 | | |
| 22 | | |
| 23 | | |
| 24 | | |
| 25 | B | C |
| 26 | A | C |
| 27 | A | D |
| 28 | B | C |
| 29 | A | C |
| 30 | A | C |
| 31 | A | C |
| 32 | B | C |
| 33 | A | D |
| 34 | A | C |
| 35 | B | D |
| 36 | B | D |
| 37 | A | C |
| 38 | A | C |
| 39 | A | C |
| 40 | A | C |
| 41 | B | D |
| 42 | B | D |
| 43 | B | D |
| 44 | B | D |
| 45 | B | D |
| 46 | B | D |
| 47 | B | D |
| 48 | C | D |
| 49 | B | D |
| 50 | B | D |
| 51 | B | D |
| 52 | A | D |
| 53 | A | D |
| 54 | A | D |
| 55 | | |
| 56 | | |
| 57 | | |
| 58 | | |
| 59 | | |
| 60 | | |
| 61 | | |
| 62 | | |
| 63 | | |
| 64 | B | D |
| 65 | B | D |
| 66 | B | D |
| 67 | B | D |
| 68 | B | D |
| 69 | A | D |
| 70 | B | D |
| 71 | B | D |
| 72 | B | D |
| 73 | B | D |
| 74 | C | D |
| 75 | A | D |
| 76 | A | D |
| 77 | A | D |
| 78 | B | D |
| 79 | B | D |
| 80 | A | D |
| 81 | A | D |
| 82 | A | D |
| 83 | A | D |
| 84 | | |
| 85 | | |
| 86 | | |
| 87 | | |

Additionally, some ASO were assessed for in vitro potency in HBV-infected primary human hepatocytes (PHHs). Table 3 below provides exemplary EC₅₀ and CC₅₀ data from these experiments.

TABLE 3
EC₅₀ and CC₅₀ in HBV-infected PHH

| ASO# | EC₅₀ (nM) | CC₅₀ (nM) |
|---|---|---|
| 64 | C | D |
| 65 | C | D |
| 69 | C | D |

### Example 4: Melting Temperature (Tₘ)

The melting temperature of several of the exemplary ASOs disclosed herein was assessed, and representative results are provided below in Table 4.

TABLE 4
Melting Temperature

| ASO# | Tₘ (° C.) |
|---|---|
| 64 | 74.3 (±0.3) |
| 65 | 77.9 (±0.3) |
| 69 | 79.4 (±0.1) |

### Example 5: Improvement in In Vivo Potency

ASOs with the disclosed chemistries were synthesized on ABI 394 and Expedite 8909 synthesizers using standard phosphoramidite chemistry. In vitro screening of ASOs was carried out in HepG2.2.15 cells using HBsAg release assay as discussed above. Certain ASOs were chosen for N-Acetylgalactosamine (GalNac) conjugation and tested at 1×5 mg/kg for a single dose in the adeno-associated virus (AAV)-HBV mouse model.

Table 5 shows exemplary HBsAg Nadir with 1×5 mg/kg QW compared to ASO 59. In these ASO in HBx region, targeting all HBV transcripts including HBx, a single replacement of a lnA with nmlnA improved the nadir for HBsAg.

TABLE 5
HBsAg Nadir

| ASO # | HBsAg Nadir with 1 × 5 mg/kg |
|---|---|
| 84 | −0.63 log10 IU/ML |
| 85 | −0.66 log10 IU/ML |
| 86 | −0.48 log10 IU/ML |
| 87 | −0.44 log10 IU/ML |

The FIGURE shows an exemplary side-by-side comparison of ASO 59 and ASO 87, with the latter containing a nmlnA and the former containing a lnA. As can be seen in the FIGURE, the addition of lnmmnA resulted in an improvement in potency.

TABLE 6
Target Gene Sequences

| SEQ ID | | |
|---|---|---|
| NO: | Description | Sequence |
| 89 | Hepatitis B | CTCCACCACTTTCCACCAAACTCTTCAAGATCCCAGAGTCAGGGCCCTG |
| | virus | TACTTTCCTGCTGGTGGCTCAAGTTCCGGAACAGTAAACCCTGCTCCGA |
| | (Genbank | CTACTGCCTCTCCCATATCGTCAATCTTCTCGAGGACTGGGGACCCTGT |
| | Accession | ACCGAATATGGAGAGCACCACATCAGGATTCCTAGGACCCCTGCTCGT |
| | No. | GTTACAGGCGGGGTTTTTCTTGTTGACAAGAATCCTCACAATACCACAG |
| | KC315400.1) | AGTCTAGACTCGTGGTGGACTTCTCTCAATTTTCTAGGGGGAGCACCCA |
| | | CGTGTCCTGGCCAAAATTTGCAGTCCCCAACCTCCAATCACTCACCAAC |
| | | CTCTTGTCCTCCAATTTGTCCTGGTTATCGCTGGATGTGTCTGCGGCGTT |
| | | TTATCATCTTCCTCTTCATCCTGCTGCTATGCCTCATCTTCTTGTTGGTTC |
| | | TTCTGGACTACCAAGGTATGTTGCCCGTTTGTCCTCTACTTCCAGGAAC |
| | | ATCAACTACCAGCACCGGACCATGCAAAACCTGCACAACTACTGCTCA |
| | | AGGGACCTCTATGTTTCCCTCATGTTGCTGTACAAAACCTACGGACGGA |
| | | AACTGCACCTGTATTCCCATCCCATCATCTTGGGCTTTCGCAAAATACC |
| | | TATGGGAGTGGGCCTCAGTCCGTTTCTCTTGGCTCAGTTTACTAGTGCC |
| | | ATTTGTTCAGTGGTTCGTAGGGCTTTCCCCCACTGTCTGGCTTTCAGTTA |
| | | TATGGATGATGTGGTTTTGGGGGCCAAGTCTGTACAACATCTTGAGTCC |
| | | CTTTATACCGCTGTTACCAATTTTCTTTTATCTTTGGGTATACATTTAAA |
| | | CCCTCACAAAACAAAAAGATGGGGATATTCCCTTAACTTCATGGGATAT |
| | | GTAATTGGGAGTTGGGGCACTTTGCCTCAGGAACATATTGTACAAAAA |
| | | ATCAAGCAATGTTTTAGGAAACTTCCTGTAAACAGGCCTATTGATTGGA |
| | | AAGTATGTCAACRAATTGTGGGTCTTTTGGGGTTTGCCGCCCCTTTCAC |
| | | GCAATGTGGATATCCTGCTTTAATGCCTTTATATGCATGTATACAAGCT |
| | | AAGCAGGCTTTTACTTTCTCGCCAACTTACAAGGCCTTTCTGTGTAAAC |
| | | AATATCTGAACCTTTACCCCGTTGCTCGGCAACGGTCAGGTCTTTGCCA |
| | | AGTGTTTGCTGACGCAACCCCCACTGGTTGGGGCTTGGCCATAGGCCAT |
| | | CAGCGCATGCGTGGAACCTTTGTGGCTCCTCTGCCGATCCATACTGCGG |
| | | AACTCCTAGCAGCTTGTTTTGCTCGCAGCCGGTCTGGAGCAAAACTTAT |
| | | CGGCACCGACAACTCTGTTGTCCTCTCTCGGAAATACACCTCCTTTCCA |
| | | TGGCTGCTAGGATGTGCTGCCAACTGGATCCTGCGCGGGACGTCCTTTG |
| | | TCTACGTCCCGTCGGCGCTGAATCCCGCGGACGACCCATCTCGGGGCCG |
| | | TTTGGGACTCTACCGTCCCCTTCTGCGTCTGCCGTTCCGCCCGACCACG |
| | | GGGCGCACCTCTCTTTACGCGGTCTCCCCGTCTGTGCCTTCTCATCTGCC |
| | | GGACCGTGTGCACTTCGCTTCACCTCTGCACGTCGCATGGAGACCACCG |
| | | TGAACGCCCACGGGAACCTGCCCAAGGTCTTGCATAAGAGGACTCTTG |
| | | GACTTTCAGCAATGTCAACGACCGACCTTGAGGCATACTTCAAAGACTG |
| | | TGTGTTTACTGAGTGGGAGGAGTTGGGGGAGGAGGTTAGGTTAAAGGT |
| | | CTTTGTACTAGGAGGCTGTAGGCATAAATTGGTGTGTTCACCAGCACCA |
| | | TGCAACTTTTTCACCTCTGCCTAATCATCTCATGTTCATGTCCTACTGTT |
| | | CAAGCCTCCAAGCTGTGCCTTGGGTGGCTTTGGGGCATGGACATTGACC |
| | | CGTATAAAGAATTTGGAGCTTCTGTGGAGTTACTCTCTTTTTTGCCTTCT |
| | | GACTTCTTTCCTTCTATTCGAGATCTCCTCGACACCGCCTCTGCTCTGTA |
| | | TCGGGAGGCCTTAGAGTCTCCGGAACATTGTTCACCTCACCATACGGCA |
| | | CTCAGGCAAGCAATTCTGTGTTGGGGTGAGTTAATGAATCTAGCCACCT |
| | | GGGTGGGAAGTAATTTGGAAGATCCAGCATCCAGGGAATTAGTAGTCA |
| | | GCTATGTCAACGTTAATATGGGCCTAAAAATCAGACAACTATTGTGGTT |
| | | TCACATTTCCTGTCTTACTTTTGGGAGAGAAACTGTTCTTGAATATTTGG |
| | | TGTCTTTTGGAGTGTGGATTCGCACTCCTCCTGCATATAGACCACAAAA |
| | | TGCCCCTATCTTATCAACACTTCCGGAAACTACTGTTGTTAGACGAAGA |
| | | GGCAGGTCCCCTAGAAGAAGAACTCCCTCGCCTCGCAGACGAAGGTCT |
| | | CAATCGCCGCGTCGCAGAAGATCTCAATCTCGGGAATCTCAATGTTAGT |
| | | ATTCCTTGGACACATAAGGTGGGAAACTTTACGGGGCTTTATTCTTCTA |
| | | CGGTACCTTGCTTTAATCCTAAATGGCAAACTCCTTCTTTTCCTGACATT |
| | | CATTTGCAGGAGGACATTGTTGATAGATGTAAGCAATTTGTGGGGCCCC |
| | | TTACAGTAAATGAAAACAGGAGACTTAAATTAATTATGCCTGCTAGGTT |
| | | TTATCCCAATGTTACTAAATATTTGCCCTTAGATAAAGGGATCAAACCG |
| | | TATTATCCAGAGTATGTAGTTAATCATTACTTCCAGACGCGACATTATT |
| | | TACACACTCTTTGGAAGGCGGGGATCTTATATAAAAGAGAGTCCACAC |
| | | GTAGCGCCTCATTTTGCGGGTCACCATATTCTTGGGAACAAGATCTACA |
| | | GCATGGGAGGTTGGTCTTCCAAACCTCGAAAAGGCATGGGGACAAATC |
| | | TTTCTGTCCCCAATCCCCTGGGATTCTTCCCCGATCATCAGTTGGACCCT |
| | | GCATTCAAAGCCAACTCAGAAAATCCAGATTGGGACCTCAACCCACAC |
| | | AAGGACAACTGGCCGGACGCCAACAAGGTGGGAGTGGGAGCATTCGG |
| | | GCCAGGGTTCACCCCTCCTCATGGGGGACTGTTGGGGTGGAGCCCTCAG |
| | | GCTCAGGGCATATTCACAACAGTGCCAGCAGCTCCTCCTCCTGCCTCCA |
| | | CCAATCGGCAGTCAGGAAGGCAGCCTACTCCCTTCTCTCCACCTCTAAG |
| | | AGACACTCATCCTCAGGCCATGCAGTGGAA |
| 90 | Hepatitis B | AATTCCACAACCTTTCACCAAACTCTGCAAGATCCCAGAGTGAGAGGC |
| | virus | CTGTATTTCCCTGCTGGTGGCTCCAGTTCAGGAGCAGTAAACCCTGTTC |
| | (Genbank | CGACTACTGCCTCTCCCTTATCGTCAATCTTCTCGAGGATTGGGGACCC |
| | Accession | TGCGCTGAACATGGAGAACATCACATCAGGATTCCTAGGACCCCTTCTC |
| | No. | GTGTTACAGGCGGGGTTTTTCTTGTTGACAAGAATCCTCACAATACCGC |
| | U95551.1) | AGAGTCTAGACTCGTGGTGGACTTCTCTCAATTTTCTAGGGGGAACTAC |
| | | CGTGTGTCTTGGCCAAAATTCGCAGTCCCCAACCTCCAATCACTCACCA |
| | | ACCTCCTGTCCTCCAACTTGTCCTGGTTATCGCTGGATGTGTCTGCGGCG |
| | | TTTTATCATCTTCCTCTTCATCCTGCTGCTATGCCTCATCTTCTTGTTGGT |
| | | TCTTCTGGACTATCAAGGTATGTTGCCCGTTTGTCCTCTAATTCCAGGAT |
| | | CCTCAACCACCAGCACGGGACCATGCCGAACCTGCATGACTACTGCTC |
| | | AAGGAACCTCTATGTATCCCTCCTGTTGCTGTACCAAACCTTCGGACGG |
| | | AAATTGCACCTGTATTCCCATCCCATCATCCTGGGCTTTCGGAAAATTC |
| | | CTATGGGAGTGGGCCTCAGCCCGTTTCTCCTGGCTCAGTTTACTAGTGC |
| | | CATTTGTTCAGTGGTTCGTAGGGCTTTCCCCCACTGTTTGGCTTTCAGTT |
| | | ATATGGATGATGTGGTATTGGGGGCCAAGTCTGTACAGCATCTTGAGTC |
| | | CCTTTTTACCGCTGTTACCAATTTTCTTTTGTCTTTGGGTATACATTTAA |
| | | ACCCTAACAAAACAAAGAGATGGGGTTACTCTCTGAATTTTATGGGTTA |
| | | TGTCATTGGAAGTTATGGGTCCTTGCCACAAGAACACATCATACAAAA |
| | | AATCAAAGAATGTTTTAGAAAACTTCCTATTAACAGGCCTATTGATTGG |
| | | AAAGTATGTCAACGAATTGTGGGTCTTTTGGGTTTTGCTGCCCCATTTA |
| | | CACAATGTGGTTATCCTGCGTTAATGCCCTTGTATGCATGTATTCAATCT |
| | | AAGCAGGCTTTCACTTTCTCGCCAACTTACAAGGCCTTTCTGTGTAAAC |
| | | AATACCTGAACCTTTACCCCGTTGCCCGGCAACGGCCAGGTCTGTGCCA |
| | | AGTGTTTGCTGACGCAACCCCCACTGGCTGGGGCTTGGTCATGGGCCAT |
| | | CAGCGCGTGCGTGGAACCTTTTCGGCTCCTCTGCCGATCCATACTGCGG |
| | | AACTCCTAGCCGCTTGTTTTGCTCGCAGCAGGTCTGGAGCAAACATTAT |
| | | CGGGACTGATAACTCTGTTGTCCTCTCCCGCAAATATACATCGTATCCA |
| | | TGGCTGCTAGGCTGTGCTGCCAACTGGATCCTGCGCGGGACGTCCTTTG |
| | | TTTACGTCCCGTCGGCGCTGAATCCTGCGGACGACCCTTCTCGGGGTCG |
| | | CTTGGGACTCTCTCGTCCCCTTCTCCGTCTGCCGTTCCGACCGACCACGG |
| | | GGCGCACCTCTCTTTACGCGGACTCCCCGTCTGTGCCTTCTCATCTGCCG |
| | | GACCGTGTGCACTTCGCTTCACCTCTGCACGTCGCATGGAGACCACCGT |
| | | GAACGCCCACCGAATGTTGCCCAAGGTCTTACATAAGAGGACTCTTGG |
| | | ACTCTCTGCAATGTCAACGACCGACCTTGAGGCATACTTCAAAGACTGT |
| | | TTGTTTAAAGACTGGGAGGAGTTGGGGGAGGAGATTAGATTAAAGGTC |
| | | TTTGTACTAGGAGGCTGTAGGCATAAATTGGTCTGCGCACCAGCACCAT |
| | | GCAACTTTTTCACCTCTGCCTAATCATCTCTTGTTCATGTCCTACTGTTC |
| | | AAGCCTCCAAGCTGTGCCTTGGGTGGCTTTGGGGCATGGACATCGACCC |
| | | TTATAAAGAATTTGGAGCTACTGTGGAGTTACTCTCGTTTTTGCCTTCTG |
| | | ACTTCTTTCCTTCAGTACGAGATCTTCTAGATACCGCCTCAGCTCTGTAT |
| | | CGGGAAGCCTTAGAGTCTCCTGAGCATTGTTCACCTCACCATACTGCAC |
| | | TCAGGCAAGCAATTCTTTGCTGGGGGGAACTAATGACTCTAGCTACCTG |
| | | GGTGGGTGTTAATTTGGAAGATCCAGCATCTAGAGACCTAGTAGTCAGT |
| | | TATGTCAACACTAATATGGGCCTAAAGTTCAGGCAACTCTTGTGGTTTC |
| | | ACATTTCTTGTCTCACTTTTGGAAGAGAAACCGTTATAGAGTATTTGGT |
| | | GTCTTTCGGAGTGTGGATTCGCACTCCTCCAGCTTATAGACCACCAAAT |
| | | GCCCCTATCCTATCAACACTTCCGGAAACTACTGTTGTTAGACGACGAG |
| | | GCAGGTCCCCTAGAAGAAGAACTCCCTCGCCTCGCAGACGAAGGTCTC |
| | | AATCGCCGCGTCGCAGAAGATCTCAATCTCGGGAACCTCAATGTTAGTA |
| | | TTCCTTGGACTCATAAGGTGGGGAACTTTACTGGTCTTTATTCTTCTACT |
| | | GTACCTGTCTTTAATCCTCATTGGAAAACACCATCTTTTCCTAATATACA |
| | | TTTACACCAAGACATTATCAAAAAATGTGAACAGTTTGTAGGCCCACTT |
| | | ACAGTTAATGAGAAAAGAAGATTGCAATTGATTATGCCTGCTAGGTTTT |
| | | ATCCAAAGGTTACCAAATATTTACCATTGGATAAGGGTATTAAACCTTA |
| | | TTATCCAGAACATCTAGTTAATCATTACTTCCAAACTAGACACTATTTA |
| | | CACACTCTATGGAAGGCGGGTATATTATATAAGAGAGAAACAACACAT |
| | | AGCGCCTCATTTTGTGGGTCACCATATTCTTGGGAACAAGATCTACAGC |
| | | ATGGGGCAGAATCTTTCCACCAGCAATCCTCTGGGATTCTTTCCCGACC |
| | | ACCAGTTGGATCCAGCCTTCAGAGCAAACACAGCAAATCCAGATTGGG |
| | | ACTTCAATCCCAACAAGGACACCTGGCCAGACGCCAACAAGGTAGGAG |
| | | CTGGAGCATTCGGGCTGGGTTTCACCCCACCGCACGGAGGCCTTTTGGG |
| | | GTGGAGCCCTCAGGCTCAGGGCATACTACAAACTTTGCCAGCAAATCC |
| | | GCCTCCTGCCTCCACCAATCGCCAGACAGGAAGGCAGCCTACCCCGCT |
| | | GTCTCCACCTTTGAGAAACACTCATCCTCAGGCCATGCAGTGG |
| 91 | 17β-HSD | ATGAACATCATCCTAGAAATCCTTCTGCTTCTGATCACCATCATCTACTC |
| | type 13 | CTACTTGGAGTCGTTGGTGAAGTTTTTCATTCCTCAGAGGAGAAAATCT |
| | coding | GTGGCTGGGGAGATTGTTCTCATTACTGGAGCTGGGCATGGAATAGGC |
| | sequence | AGGCAGACTACTTATGAATTTGCAAAACGACAGAGCATATTGGTTCTGT |
| | (Genbank | GGGATATTAATAAGCGCGGTGTGGAGGAAACTGCAGCTGAGTGCCGAA |
| | Accession | AACTAGGCGTCACTGCGCATGCGTATGTGGTAGACTGCAGCAACAGAG |
| | No. NM | AAGAGATCTATCGCTCTCTAAATCAGGTGAAGAAAGAAGTGGGTGATG |
| | 178135.5) | TAACAATCGTGGTGAATAATGCTGGGACAGTATATCCAGCCGATCTTCT |
| | (nucleotides | CAGCACCAAGGATGAAGAGATTACCAAGACATTTGAGGTCAACATCCT |
| | 42 to 944) | AGGACATTTTTGGATCACAAAAGCACTTCTTCCATCGATGATGGAGAGA |
| | | AATCATGGCCACATCGTCACAGTGGCTTCAGTGTGCGGCCACGAAGGG |
| | | ATTCCTTACCTCATCCCATATTGTTCCAGCAAATTTGCCGCTGTTGGCTT |
| | | TCACAGAGGTCTGACATCAGAACTTCAGGCCTTGGGAAAAACTGGTAT |
| | | CAAAACCTCATGTCTCTGCCCAGTTTTTGTGAATACTGGGTTCACCAAA |
| | | AATCCAAGCACAAGATTATGGCCTGTATTGGAGACAGATGAAGTCGTA |
| | | AGAAGTCTGATAGATGGAATACTTACCAATAAGAAAATGATTTTTGTTC |
| | | CATCGTATATCAATATCTTTCTGAGACTACAGAAGTTTCTTCCTGAACG |
| | | CGCCTCAGCGATTTTAAATCGTATGCAGAATATTCAATTTGAAGCAGTG |
| | | GTTGGCCACAAAATCAAAATGAAATGA |
| 92 | MCJ mRNA | AGTCACTGCCGCGGCGCCTTGAGTCTCCGGGCCGCCTTGCCATGGCTGC |
| | (GenBank | CCGTGGTGTCATCGCTCCAGTTGGCGAGAGTTTGCGCTACGCTGAGTAC |
| | Accession | TTGCAGCCCTCGGCCAAACGGCCAGACGCCGACGTCGACCAGCAGAGA |
| | No. | CTGGTAAGAAGTTTGATAGCTGTAGGACTGGGTGTTGCAGCTCTTGCAT |
| | NM_013238.3) | TTGCAGGTCGCTACGCATTTCGGATCTGGAAACCTCTAGAACAAGTTAT |
| | | CACAGAAACTGCAAAGAAGATTTCAACTCCTAGCTTTTCATCCTACTAT |
| | | AAAGGAGGATTTGAACAGAAAATGAGTAGGCGAGAAGCTGGTCTTATT |
| | | TTAGGTGTAAGCCCATCTGCTGGCAAGGCTAAGATTAGAACAGCTCAT |
| | | AGGAGAGTCATGATTTTGAATCACCCAGATAAAGGTGGATCTCCTTACG |
| | | TAGCAGCCAAAATAAATGAAGCAAAAGACTTGCTAGAAACAACCACCA |
| | | AACATTGATGCTTAAGGACCACACTGAAGGAAAAAAAAAGAGGGGAC |
| | | TTCGAAAAAAAAAAAAGCCCTGCAAAATATTCTAAAACATGGTCTTCTT |
| | | AATTTTCTATATGGATTGACCACAGTCTTATCTTCCACCATTAAGCTGTA |
| | | TAACAATAAAATGTTAATAGTCTTGCTTTTTATTATCTTTTAAAGATCTC |
| | | CTTAAATTCTATAACTGATCTTTTTTCTTATTTTGTTTGTGACATTCATAC |
| | | ATTTTTAAGATTTTTGTTATGTTCTGAATTCCCCCCTACACACACACACA |
| | | CACACACACACACACACACGTGCAAAAAATATGATCAAGAATGCAATT |
| | | GGGATTTGTGAGCAATGAGTAGACCTCTTATTGTTTATATTTGTACCCTC |
| | | ATTGTCAATTTTTTTTTAGGGAATTTGGGACTCTGCCTATATAAGGTGTT |
| | | TTAAATGTCTTGAGAACAAGCACTGGCTGATACCTCTTGGAGATATGAT |
| | | CTGAAATGTAATGGAATTTATTAAATGGTGTTTAGTAAAGTAGGGGTTA |
| | | AGGACTTGTTAAAGAACCCCACTATCTCTGAGACCCTATAGCCAAAGC |
| | | ATGAGGACTTGGAGAGCTACTAAAATGATTCAGGTTTACAAAATGAGC |
| | | CCTGTGAGGAAAGGTTGAGAGAAGTCTGAGGAGTTTGTATTTAATTATA |
| | | GTCTTCCAGTACTGTATATTCATTCATTACTCATTCTACAAATATTTATT |
| | | GACCCCTTTTGATGTGCAAGGCACTATCGTGCGTCCCCTGAGAGTTGCA |
| | | AGTATGAAGCAGTCATGGATCATGAACCAAAGGAACTTATATGTAGAG |
| | | GAAGGATAAATCACAAATAGTGAATACTGTTAGATACAGATGATATAT |
| | | TTTAAAAGTTCAAAGGAAGAAAAGAATGTGTTAAACACTGCATGAGAG |
| | | GAGGAATAAGTGGCATAGAGCTAGGCTTTAGAAAAGAAAAATATTCCG |
| | | ATACCATATGATTGGTGAGGTAAGTGTTATTCTGAGATGAGAATTAGCA |
| | | GAAATAGATATATCAATCGGAGTGATTAGAGTGCAGGGTTTCTGGAAA |
| | | GCAAGGTTTGGACAGAGTGGTCATCAAAGGCCAGCCCTGTGACTTACA |
| | | CTGCATTAAATTAATTTCTTAGAACATAGTCCCTGATCATTATCACTTTA |
| | | CTATTCCAAAGGTGAGAGAACAGATTCAGATAGAGTGCCAGCATTGTT |
| | | TCCCAGTATTCCTTTACAAATCTTGGGTTCATTCCAGGTAAACTGAACT |
| | | ACTGCATTGTTTCTATCTTAAAATACTTTTTAGATATCCTAGATGCATCT |
| | | TTCAACTTCTAACATTCTGTAGTTTAGGAGTTCTCAACCTTGGCATTATT |
| | | GACATGTTAGGCCAAATAATTTTTTTTGTGGGAGGTCTCTTGTGCGTTTT |
| | | AGATGATTAGCAATAATCCCTGACCTGTTATCTACTAAAGACTAGTCGT |
| | | TTCTCATCAGTTGTGACAACAAAAATGGTTCCAGATATTGCCAAATGCC |
| | | CTTTAGAGGACAGTAATCGCCCCCAGTTGAGAACCATTTCAGTAAAACT |
| | | TTAATTACTATTTTTTCTTTTGGTTTATAAAATAATGATCCTGAATTAAA |
| | | TTGATGGAACCTTGAAGTCGATAAAATATATTTCTTGCTTTAAAGTCCC |
| | | CATACGTGTCCTACTAATTTTCTCATGCTTTAGTGTTTTCACTTTTCTCCT |
| | | GTTATCCTTGTACCTAAGAATGCCATCCCAATCCCCAGATGTCCACCTG |
| | | CCCAAAGTCTAGGCATAGCTGAAGGCCAAGCTAAAATGTATCCCTCTTT |
| | | TTCTGGTACATGCAGCAAAAGTAATATGAATTATCAGCTTTCTGAGAGC |
| | | AGGCATTGTATCTGTCTTGTTTGGTGTTACATTGGCACCCAATAAATATT |
| | | TGTTGAGTGAATGAATAAATTCCCATAGCACTTTATTCTTCACATGGTA |
| | | CATAACTATAGGGGCTATAGCTTGGTACCTTGTGAAGCAACTCTTGGTG |
| | | TAACATACCTTATTTCTCATACTAAAATGCAAGAACCTTTAGAGCAAGG |
| | | ATCTTGCCATTCATCTTTGTAACCTCTTTACTCTGGAGCACTTGCATTTA |
| | | GCAGGCATCATAAAGTTTTACGTACCAAGAAAATGTTGCTGTTTTCTGA |
| | | ATACTATGCATCAAAAAATGTTACCACTAATTTTTAAAGCTCTGCTAAG |
| | | GAATATTGGGGCACCCTCAGATGCACCTTTTAATTGATGTCATATTTTC |
| | | CTAATCCATACTTTATTCATGAGAATTTGAGTCACCCCAGCATTAGCTT |
| | | GGAATTTCCTTATTTCCCATTTGCTTTGCAGGTGCCTTGGAGTCAGATCT |
| | | GGTTTTGAATACTATCTTCCTGTTATGTGATCTTGGGCAGTTACTTAATT |
| | | TTCTAGTCAATAACCCGTATCTATAAAATAGAGAAAATAATCCTACACA |
| | | CCGGGGCCTGTTGTGGGGGGGGGAGAGGGGGGAGGGATCGCATTTGGA |
| | | GATATACTAATGTAAATGACAAGTTAATTGGTGCAGCACACCAACATG |
| | | GCTCATGTCTACATATGTAACAAACCTGCACGTTGTGCACATGTGCCCT |
| | | AGAACTTAAAGTATAATAAAAAGAAATTTTAAAAAATCCTGTCAAATA |
| | | AGGTTATAGTAGAGAATAAGGATGTGTAAAGCATTTAGTCACGTAAAT |
| | | GCTTAAAAAAATGTAATTTTTACTTCTTTCACTGCCTCATTTAATTAGTT |
| | | TTATCTTTAATAATACCTTGGATTCAGGGTAAAGTTTCAGTTATGTCCCA |
| | | GTAATCATTTATTTTACCCTCGAATCTGCAATTTGGATAGAACATGGTG |
| | | GGGACAGCTCGTCTCTATTCCTTGCAGCATTAACAGGCTGGAGGCACCA |
| | | CTTCTCTGGCCAGCAAGTTGGGCCTGGTTGTTGGCTGAGAGCCTCAGTT |
| | | CCTTTCTGCACAGGTTCCTCTTTACATAGGCTTCTCAACAGGGCTACTA |
| | | GAGCATCGTCACCATAGCAGCTGTCTTATAACAGAGAGTGGTCGGTCTG |
| | | AGAGACAAAAAATGGAAGCTGCCAAATTGTTCTGGGTCTGGAAACTGT |
| | | CAGGGCATCACTTGTGCCATATTCAGTTGGCCTAAGAATTACAGAGCCT |
| | | GCCTCGATTCAAAGGGAGAGGATAGAGAGGACTGAAGGAATCAGTGCT |
| | | CATCTTTAATATGCAGCAGGACAGGTTTGGGATTTTTTTTCCCCCTTGAG |
| | | TCTGTGAAGGCATTACTTAAGAACAAAGTCAGGCATGTATAATTGAACT |
| | | ACAGTTACTTGAAATATAAGCCCAGAAAGTTTCAGATAATAAATACAA |
| | | CTATTTTTCTGCTGTTACCCTTGTACCTAAAGATGCCATCCTAATCCCCA |
| | | GATCTCCACAACTATACCTACATAGTAGAAGGTTAAAATGTATCCCTCT |
| | | TTTTCTGGTGCATCCAGCAAAAGTAATATCATGAATTATGAGCTCTCTG |
| | | AGAGCAAGGATCATATCAGTCTTGTTTATTGTTGCAGTGAACAAGTACA |
| | | GTTGCAGATATTCAGGAGTAATTATCTAAATGGCAGTAGGCTTATAAAA |
| | | CTGAATTTTCACCAGCCACACCCTCCCCCCAACTCCTTATCTGTAAAAA |
| | | GCTTATTTGAGTGGTTACCTGTCTTCAGTAAAGATTGCGCTTGCATATTT |
| | | GCTGTCATTGCATATTCTGCTTAATTAAGCTCTGTTGATATTGCAGTTTC |
| | | TGTGCATACTTACATCTTAGATGCAATCTGAGGGCCTAGGAAGGCCTTT |
| | | TAAAAATAAAACAATTCCGATTGCAGAGAAAGTGTAAGTCAAGGACAG |
| | | TTAATTCAAGGGGAACATAGAAAGCTATTTAGATTTTAGTTGATGGTGC |
| | | CAGTCTTCAGCGTAAAGTCAAAAGTGGAGGGAAGTTTAGTAAGGAAAA |
| | | AATGTTGGGCTTGGAATACATTGTTTAGTCTTCAAAGCACTTTACTTTTT |
| | | ATGAAATATATTTTAGACATTCAGCAAATATTGAATACTTACTATATCA |
| | | GGCAGTAAAGATATAAATTCATTCTTAAAATGTGCAACATGTTCAAACT |
| | | GAAAAAAATACATTCTTAAACAGGAAACTTTTTCCTTCATACTTTTTAA |
| | | TTAACAAGACATATAAGAGTTGCATTAATGGGCGTGCTTATGATTGATC |
| | | ACCCAGCAGCATCATTAGAAATAATATATTTTATTCATGTGCAGAAATC |
| | | TTTTGGTTGTCCTGGGGAACCTTGAACACAGAAAAGAGCTTTTATTGAT |
| | | AAGGTAATTGAACACACTTGACAATTAGCTTAATATGGTTTAATACCAT |
| | | TTGTGGGAGAAGATGAATCAGCCAGGCTCTTTACGTCAAGAATATGAA |
| | | GTTTCTCTTGAGTCAACCAACTTAAGATGAGCTACGGAGACTGCAGTGA |
| | | AAAGTTAAATATCCAAGTACACCAGCCAATTTCACACAGTGGAACCAT |
| | | GCTGTCCTCGGGCACCCTGCACCTCGCCCAACAGTCATCAACTAGATGG |
| | | AGGCTCCTGGCTGCAAGGAGGATTTGATGGGAATGAGTAAATGTGTCA |
| | | GCATAGTCCGTCCCTTCTAATGGAAAAGCAACCCAAAGAGCAAATCCT |
| | | ATTAATGGCTGGATCAGTATCATCTACTTGTCAAAAACATTCCATGAAT |
| | | TATGAGTCAAAATTTTATTTATGGTGGCATTACACACATTAAGAGATGA |
| | | GGACTTCTGTTAGCATAATTTATTAGCTGGAAAAGTTGAGAAGGTTCTC |
| | | TGGACTCATTTTTATAGGTGGAACCTAAGTGATCTGGATAATTGCCCAC |
| | | CAGCAAAATTGCTGGGCATGGTGGACAAAGAAAATGTTCCTTCTAATG |
| | | ATTTTTTATGAGCTGAGTAGCTATTGTTCCCAGCTGAGTGCTCTTTTCCT |
| | | CTTTTTATTGTTGCTGAGCAAAAGAATTTATAAAAAGCTCTTTCTTTTGT |
| | | ATTAAAAACCCTGCTCAATTGAAATGCAAGTTCATTAAGTAATCTTCAT |
| | | TTCTCTTCCTGCCATAATAACCCTTTCCCTCTCTGTTCGATTCAACAGTA |
| | | TCTAGCAGCACTGCTCCAAATTTTAAGTCTGAACAGACTATATTACATA |
| | | GATGTAGAGAAATACTCAATCTTCAGCATTAAGAGGGAGCTTAATTTCA |
| | | CACGGGTGGAATATGATCACTCAGGCTAGATGTTGGCCATAAATTTCAA |
| | | ATTAGTATCTCAACTTAGCAGGGGGGATCAACAGTGGCAAACTTCAATT |
| | | ATGACAGGATAAAAATCACATAGAGATATTGGTTCAATATGGACATCT |
| | | AAACTATAATGCTAAAAGCCAATAATTAGAATAAGTTCATTTTAAGAA |
| | | AAGCATTAATAATATTAGCTAACGTTTAGTACCTGTGCCAAACATTCTA |
| | | CCTATGTTACCTTGATTTTCATAGCCAGCCTAAGAGGTACTATTATGTAT |
| | | CCCCATTTTACAGGTTAAGAAACAGGCTCAGAGGAGTTTAGGATCTTTT |
| | | CCAAGATTACATAGCCAGTAAGTGGTGGCACTAGGAACCAAATTCAGA |
| | | CTCTGAATCGCATGCTGTTTATATTATATTGCACTCATTCTAAATATGTG |
| | | GGAATCAGAATGAAGGGGCTTGTATGACTTTTGGCTCATTTTTTGATGC |
| | | ATGTGACCTGGGATTATAAATGTGAAATTAGGTTTACGAAAGGATCCA |
| | | GTGTCATTGTGCATCATGGGCAAGGAGTACCTAATCTCTTTAATTCTTC |
| | | CCTGGAAGCTTACGATGTCCATCCAAGTGCACATAGCAAAAGTTCTGTT |
| | | GTAAAGTTTAGCAGAGTGACTTTCTTTGACTCAGAGTGATGACGGAGG |
| | | AAGCTTTGATAAGATTTTATCTGAAATGTTCATGGACAAGAGCTTTCAA |
| | | GGAGAACATCCAGAGCAAGGTTCTGAAGACAGCTCATGAAGGTGAAGC |
| | | AGCAGACCTGGCACAAGAAATGAAGAGAGAGCTCAGTGTATTAAAGAT |
| | | GAAAACAAGAAAACCGAATATATTGAAAGGAGCAGAGAGGCAATGAA |
| | | AACAAGACAACTGAAATGAGGTAACTTGCAGCAATTGAAAGGGAATTT |
| | | CAGTACTTTTATAGAATTCTTAAAAATTGTTTCCTGCTGTTTATTTTCAA |
| | | TTTTGAACAGGGTTATTTGTCCATGCCATACTTTTTTTGCCAAATTCCAA |
| | | AATTGTGTATAGTTCTATAGTTGTCTGGTGGAGTCAATGGAACTTTAGT |
| | | TACCAGTCTAAGAATGTGTCTTTGAGATTGTCCAGTTAATTCTCTATTTC |
| | | CAGTAGCTGTAATAAATGGTGAAAAGGTTTCTGACTCCTGGAGAAAGT |
| | | TTCTAACTCCTTATGACTAATATTCATAACAGACTTGTGAGTTCCTTGAA |
| | | CATGGATACACCTATATGCAAGAGTGTATTCCAAAGCTAACTCAGTGAT |
| | | CTTTCCATTTATCTATTCTTGGATTAGTGGTGCCTTTGCTCTTTCCTTCTG |
| | | TAAATGTGAATAGTTAAGAGTTGACTGCAGAAGTGTTTACACTTTGGCT |
| | | TCCATGCCTCTGGAATGTTTGTGCTTTGGTGGTGAGATGTGAGACTATA |
| | | TTTGTATAGTCTGCATCTCTCAGGCTGCCCCAGAATGTTGTACAGTGCA |
| | | GTGCTGAAGAAAGCAGCAGGTACACACAGAAATGCAGCCTTTCCTGGT |
| | | TAACCCTGCTTGGATCTGAGTTACACTTTGTTTCCTGACTTCTTGGGACT |
| | | TAGGTAATCAGTTTGCCTTCTACTCTATCTCATTTTGTACTCGCTTACAT |
| | | ACTACATTCTTGTTTGGGCTTTCGTTTCTTCTTGTAAGCAGAGATTTTTT |
| | | AAAATCCAATATGTGAAAATACGGATGCACTACAATTAAATAAATAAA |
| | | ATGCTGTTGTGTTTGTTTTGCTTTAAAATTGTAAAGGATAAACAATAAG |
| | | ATAGTTTTATCTATGTGGTTTTCCCGATGCAGTTAAAATAAAACCTAAT |
| | | CTGCTAAAATTGAA |
| 93 | TAZ | GCTTTCCGGCGGTTGCACCGGGCCGGGGTGCCAGCGCCCGCCTTCCCGT |
| | (GenBank | TTCCTCCCGTTCCGCAGCGCGCCCACGGCCTGTGACCCCGGCGACCGCT |
| | Accession | CCCCAGTGACGAGAGAGCGGGGCCGGGCGCTGCTCCGGCCTGACCTGC |
| | No. | GAAGGGACCTCGGTCCAGTCCCCTGTTGCGCCGCGCCCCCGTCCGTCCG |
| | NM_000116.5) | TGCGCGGGCCAGTCAGGGGCCAGTGTCTCGAGCGGTCGAGGTCGCAGA |
| | | CCTAGAGGCGCCCCACAGGCCGGCCCGGGGCGCTGGGAGCGCCGGCCG |
| | | CGGGCCGGGTGGGGATGCCTCTGCACGTGAAGTGGCCGTTCCCCGCGG |
| | | TGCCGCCGCTCACCTGGACCCTGGCCAGCAGCGTCGTCATGGGCTTGGT |
| | | GGGCACCTACAGCTGCTTCTGGACCAAGTACATGAACCACCTGACCGT |
| | | GCACAACAGGGAGGTGCTGTACGAGCTCATCGAGAAGCGAGGCCCGGC |
| | | CACGCCCCTCATCACCGTGTCCAATCACCAGTCCTGCATGGACGACCCT |
| | | CATCTCTGGGGGATCCTGAAACTCCGCCACATCTGGAACCTGAAGTTGA |
| | | TGCGTTGGACCCCTGCAGCTGCAGACATCTGCTTCACCAAGGAGCTACA |
| | | CTCCCACTTCTTCAGCTTGGGCAAGTGTGTGCCTGTGTGCCGAGGAGCA |
| | | GAATTTTTCCAAGCAGAGAATGAGGGGAAAGGTGTTCTAGACACAGGC |
| | | AGGCACATGCCAGGTGCTGGAAAAAGAAGAGAGAAAGGAGATGGCGT |
| | | CTACCAGAAGGGGATGGACTTCATTTTGGAGAAGCTCAACCATGGGGA |
| | | CTGGGTGCATATCTTCCCAGAAGGGAAAGTGAACATGAGTTCCGAATT |
| | | CCTGCGTTTCAAGTGGGGAATCGGGCGCCTGATTGCTGAGTGTCATCTC |
| | | AACCCCATCATCCTGCCCCTGTGGCATGTCGGAATGAATGACGTCCTTC |
| | | CTAACAGTCCGCCCTACTTCCCCCGCTTTGGACAGAAAATCACTGTGCT |
| | | GATCGGGAAGCCCTTCAGTGCCCTGCCTGTACTCGAGCGGCTCCGGGCG |
| | | GAGAACAAGTCGGCTGTGGAGATGCGGAAAGCCCTGACGGACTTCATT |
| | | CAAGAGGAATTCCAGCATCTGAAGACTCAGGCAGAGCAGCTCCACAAC |
| | | CACCTCCAGCCTGGGAGATAGGCCTTGCTTGCTGCCTTCTGGATTCTTG |
| | | GCCCGCACAGAGCTGGGGCTGAGGGATGGACTGATGCTTTTAGCTCAA |
| | | ACGTGGCTTTTAGACAGATTTGTTCATAGACCCTCTCAAGTGCCCTCTC |
| | | CGAGCTGGTAGGCATTCCAGCTCCTCCGTGCTTCCTCAGTTACACAAAG |
| | | GACCTCAGCTGCTTCTCCCACTTGGCCAAGCAGGGAGGAAGAAGCTTA |
| | | GGCAGGGCTCTCTTTCCTTCTTGCCTTCAGATGTTCTCTCCCAGGGGCTG |
| | | GCTTCAGGAGGGAGCATAGAAGGCAGGTGAGCAACCAGTTGGCTAGGG |
| | | GAGCAGGGGGCCCACCAGAGCTGTGGAGAGGGGACCCTAAGACTCCTC |
| | | GGCCTGGCTCCTACCCACCGCCCTTGCCGAACCAGGAGCTGCTCACTAC |
| | | CTCCTCAGGGATGGCCGTTGGCCACGTCTTCCTTCTGCCTGAGCTTCCCC |
| | | CCCACCACAGGCCCTTTCCTCAGGCAAGGTCTGGCCTCAGGTGGGCCGC |
| | | AGGCGGGAAAAGCAGCCCTTGGCCAGAAGTCAAGCCCAGCCACGTGGA |
| | | GCCTAGAGTGAGGGCCTGAGGTCTGGCTGCTTGCCCCCATGCTGGCGCC |
| | | AACAACTTCTCCATCCTTTCTGCCTCTCAACATCACTTGAATCCTAGGGC |
| | | CTGGGTTTTCATGTTTTTGAAACAGAACCATAAAGCATATGTGTTGGCT |
| | | TGTTGTAAAA |
| 94 | ANGPTL3 | AGAAGAAAACAGTTCCACGTTGCTTGAAATTGAAAATCAAGATAAAAA |
| | (GenBank | TGTTCACAATTAAGCTCCTTCTTTTTATTGTTCCTCTAGTTATTTCCTCCA |
| | Accession | GAATTGATCAAGACAATTCATCATTTGATTCTCTATCTCCAGAGCCAAA |
| | No. | ATCAAGATTTGCTATGTTAGACGATGTAAAAATTTTAGCCAATGGCCTC |
| | NM_014495.4) | CTTCAGTTGGGACATGGTCTTAAAGACTTTGTCCATAAGACGAAGGGCC |
| | | AAATTAATGACATATTTCAAAAACTCAACATATTTGATCAGTCTTTTTA |
| | | TGATCTATCGCTGCAAACCAGTGAAATCAAAGAAGAAGAAAAGGAACT |
| | | GAGAAGAACTACATATAAACTACAAGTCAAAAATGAAGAGGTAAAGA |
| | | ATATGTCACTTGAACTCAACTCAAAACTTGAAAGCCTCCTAGAAGAAA |
| | | AAATTCTACTTCAACAAAAAGTGAAATATTTAGAAGAGCAACTAACTA |
| | | ACTTAATTCAAAATCAACCTGAAACTCCAGAACACCCAGAAGTAACTT |
| | | CACTTAAAACTTTTGTAGAAAAACAAGATAATAGCATCAAAGACCTTCT |
| | | CCAGACCGTGGAAGACCAATATAAACAATTAAACCAACAGCATAGTCA |
| | | AATAAAAGAAATAGAAAATCAGCTCAGAAGGACTAGTATTCAAGAACC |
| | | CACAGAAATTTCTCTATCTTCCAAGCCAAGAGCACCAAGAACTACTCCC |
| | | TTTCTTCAGTTGAATGAAATAAGAAATGTAAAACATGATGGCATTCCTG |
| | | CTGAATGTACCACCATTTATAACAGAGGTGAACATACAAGTGGCATGT |
| | | ATGCCATCAGACCCAGCAACTCTCAAGTTTTTCATGTCTACTGTGATGT |
| | | TATATCAGGTAGTCCATGGACATTAATTCAACATCGAATAGATGGATCA |
| | | CAAAACTTCAATGAAACGTGGGAGAACTACAAATATGGTTTTGGGAGG |
| | | CTTGATGGAGAATTTTGGTTGGGCCTAGAGAAGATATACTCCATAGTGA |
| | | AGCAATCTAATTATGTTTTACGAATTGAGTTGGAAGACTGGAAAGACA |
| | | ACAAACATTATATTGAATATTCTTTTTACTTGGGAAATCACGAAACCAA |
| | | CTATACGCTACATCTAGTTGCGATTACTGGCAATGTCCCCAATGCAATC |
| | | CCGGAAAACAAAGATTTGGTGTTTTCTACTTGGGATCACAAAGCAAAA |
| | | GGACACTTCAACTGTCCAGAGGGTTATTCAGGAGGCTGGTGGTGGCAT |
| | | GATGAGTGTGGAGAAAACAACCTAAATGGTAAATATAACAAACCAAGA |
| | | GCAAAATCTAAGCCAGAGAGGAGAAGAGGATTATCTTGGAAGTCTCAA |
| | | AATGGAAGGTTATACTCTATAAAATCAACCAAAATGTTGATCCATCCAA |
| | | CAGATTCAGAAAGCTTTGAATGAACTGAGGCAAATTTAAAAGGCAATA |
| | | ATTTAAACATTAACCTCATTCCAAGTTAATGTGGTCTAATAATCTGGTA |
| | | TTAAATCCTTAAGAGAAAGCTTGAGAAATAGATTTTTTTTATCTTAAAG |
| | | TCACTGTCTATTTAAGATTAAACATACAATCACATAACCTTAAAGAATA |
| | | CCGTTTACATTTCTCAATCAAAATTCTTATAATACTATTTGTTTTAAATT |
| | | TTGTGATGTGGGAATCAATTTTAGATGGTCACAATCTAGATTATAATCA |
| | | ATAGGTGAACTTATTAAATAACTTTTCTAAATAAAAAATTTAGAGACTT |
| | | TTATTTTAAAAGGCATCATATGAGCTAATATCACAACTTTCCCAGTTTA |
| | | AAAAACTAGTACTCTTGTTAAAACTCTAAACTTGACTAAATACAGAGG |
| | | ACTGGTAATTGTACAGTTCTTAAATGTTGTAGTATTAATTTCAAAACTA |
| | | AAAATCGTCAGCACAGAGTATGTGTAAAAATCTGTAATACAAATTTTTA |
| | | AACTGATGCTTCATTTTGCTACAAAATAATTTGGAGTAAATGTTTGATA |
| | | TGATTTATTTATGAAACCTAATGAAGCAGAATTAAATACTGTATTAAAA |
| | | TAAGTTCGCTGTCTTTAAACAAATGGAGATGACTACTAAGTCACATTGA |
| | | CTTTAACATGAGGTATCACTATACCTTATTTGTTAAAATATATACTGTAT |
| | | ACATTTTATATATTTTAACACTTAATACTATGAAAACAAATAATTGTAA |
| | | AGGAATCTTGTCAGATTACAGTAAGAATGAACATATTTGTGGCATCGA |
| | | GTTAAAGTTTATATTTCCCCTAAATATGCTGTGATTCTAATACATTCGTG |
| | | TAGGTTTTCAAGTAGAAATAAACCTCGTAACAAGTTACTGAACGTTTAA |
| | | ACAGCCTGACAAGCATGTATATATGTTTAAAATTCAATAAACAAAGAC |
| | | CCAGTCCCTAAATTATAGAAATTTAAATTATTCTTGCATGTTTATCGAC |
| | | ATCACAACAGATCCCTAAATCCCTAAATCCCTAAAGATTAGATACAAAT |
| | | TTTTTACCACAGTATCACTTGTCAGAATTTATTTTTAAATATGATTTTTT |
| | | AAAACTGCCAGTAAGAAATTTTAAATTAAACCCATTTGTTAAAGGATAT |
| | | AGTGCCCAAGTTATATGGTGACCTACCTTTGTCAATACTTAGCATTATG |
| | | TATTTCAAATTATCCAATATACATGTCATATATATTTTTATATGTCACAT |
| | | ATATAAAAGATATGTATGATCTATGTGAATCCTAAGTAAATATTTTGTT |
| | | CCAGAAAAGTACAAAATAATAAAGGTAAAAATAATCTATAATTTTCAG |
| | | GACCACAGACTAAGCTGTCGAAATTAACGCTGATTTTTTTAGGGCCAGA |
| | | ATACCAAAATGGCTCCTCTCTTCCCCCAAAATTGGACAATTTCAAATGC |
| | | AAAATAATTCATTATTTAATATATGAGTTGCTTCCTCTATTTGGTTTCC |
| 95 | DGAT2 | TGCCCCGTTGTGAGGTGATAAAGTGTTGCGCTCCGGGACGCCAGCGCC |
| | (GenBank | GCGGCTGCCGCCTCTGCTGGGGTCTAGGCTGTTTCTCTCGCGCCACCAC |
| | Accession | TGGCCGCCGGCCGCAGCTCCAGGTGTCCTAGCCGCCCAGCCTCGACGCC |
| | No. | GTCCCGGGACCCCTGTGCTCTGCGCGAAGCCCTGGCCCCGGGGGCCGG |
| | NM_001253891.1) | GGCATGGGCCAGGGGCGCGGGGTGAAGCGGCTTCCCGCGGGGCCGTGA |
| | | CTGGGCGGGCTTCAGCCATGAAGACCCTCATAGCCGCCTACTCCGGGGT |
| | | CCTGCGCGGCGAGCGTCAGGCCGAGGCTGACCGGAGCCAGCGCTCTCA |
| | | CGGAGGACCTGCGCTGTCGCGCGAGGGGTCTGGGAGATGGGGAGTGGC |
| | | CTGCAGTGCCATCCTCATGTACATATTCTGCACTGATTGCTGGCTCATC |
| | | GCTGTGCTCTACTTCACTTGGCTGGTGTTTGACTGGAACACACCCAAGA |
| | | AAGGTGGCAGGAGGTCACAGTGGGTCCGAAACTGGGCTGTGTGGCGCT |
| | | ACTTTCGAGACTACTTTCCCATCCAGCTGGTGAAGACACACAACCTGCT |
| | | GACCACCAGGAACTATATCTTTGGATACCACCCCCATGGTATCATGGGC |
| | | CTGGGTGCCTTCTGCAACTTCAGCACAGAGGCCACAGAAGTGAGCAAG |
| | | AAGTTCCCAGGCATACGGCCTTACCTGGCTACACTGGCAGGCAACTTCC |
| | | GAATGCCTGTGTTGAGGGAGTACCTGATGTCTGGAGGTATCTGCCCTGT |
| | | CAGCCGGGACACCATAGACTATTTGCTTTCAAAGAATGGGAGTGGCAA |
| | | TGCTATCATCATCGTGGTCGGGGGTGCGGCTGAGTCTCTGAGCTCCATG |
| | | CCTGGCAAGAATGCAGTCACCCTGCGGAACCGCAAGGGCTTTGTGAAA |
| | | CTGGCCCTGCGTCATGGAGCTGACCTGGTTCCCATCTACTCCTTTGGAG |
| | | AGAATGAAGTGTACAAGCAGGTGATCTTCGAGGAGGGCTCCTGGGGCC |
| | | GATGGGTCCAGAAGAAGTTCCAGAAATACATTGGTTTCGCCCCATGCAT |
| | | CTTCCATGGTCGAGGCCTCTTCTCCTCCGACACCTGGGGGCTGGTGCCC |
| | | TACTCCAAGCCCATCACCACTGTTGTGGGAGAGCCCATCACCATCCCCA |
| | | AGCTGGAGCACCCAACCCAGCAAGACATCGACCTGTACCACACCATGT |
| | | ACATGGAGGCCCTGGTGAAGCTCTTCGACAAGCACAAGACCAAGTTCG |
| | | GCCTCCCGGAGACTGAGGTCCTGGAGGTGAACTGAGCCAGCCTTCGGG |
| | | GCCAATTCCCTGGAGGAACCAGCTGCAAATCACTTTTTTGCTCTGTAAA |
| | | TTTGGAAGTGTCATGGGTGTCTGTGGGTTATTTAAAAGAAATTATAACA |
| | | ATTTTGCTAAACCATTACAATGTTAGGTCTTTTTTAAGAAGGAAAAAGT |
| | | CAGTATTTCAAGTTCTTTCACTTCCAGCTTGCCCTGTTCTAGGTGGTGGC |
| | | TAAATCTGGGCCTAATCTGGGTGGCTCAGCTAACCTCTCTTCTTCCCTTC |
| | | CTGAAGTGACAAAGGAAACTCAGTCTTCTTGGGGAAGAAGGATTGCCA |
| | | TTAGTGACTTGGACCAGTTAGATGATTCACTTTTTGCCCCTAGGGATGA |
| | | GAGGCGAAAGCCACTTCTCATACAAGCCCCTTTATTGCCACTACCCCAC |
| | | GCTCGTCTAGTCCTGAAACTGCAGGACCAGTTTCTCTGCCAAGGGGAGG |
| | | AGTTGGAGAGCACAGTTGCCCCGTTGTGTGAGGGCAGTAGTAGGCATC |
| | | TGGAATGCTCCAGTTTGATCTCCCTTCTGCCACCCCTACCTCACCCCTAG |
| | | TCACTCATATCGGAGCCTGGACTGGCCTCCAGGATGAGGATGGGGGTG |
| | | GCAATGACACCCTGCAGGGGAAAGGACTGCCCCCCATGCACCATTGCA |
| | | GGGAGGATGCCGCCACCATGAGCTAGGTGGAGTAACTGGTTTTTCTTGG |
| | | GTGGCTGATGACATGGATGCAGCACAGACTCAGCCTTGGCCTGGAGCA |
| | | CATGCTTACTGGTGGCCTCAGTTTACCTTCCCCAGATCCTAGATTCTGG |
| | | ATGTGAGGAAGAGATCCCTCTTCAGAAGGGGCCTGGCCTTCTGAGCAG |
| | | CAGATTAGTTCCAAAGCAGGTGGCCCCCGAACCCAAGCCTCACTTTTCT |
| | | GTGCCTTCCTGAGGGGGTTGGGCCGGGGAGGAAACCCAACCCTCTCCT |
| | | GTGTGTTCTGTTATCTCTTGATGAGATCATTGCACCATGTCAGACTTTTG |
| | | TATATGCCTTGAAAATAAATGAAAGTGAGAATCCTCTAAAAAAAAAAA |
| | | A |

1. An antisense oligonucleotide (ASO), comprising 14-22 nucleotide units and:
(a) a central region (B′) comprising 6 or more contiguous DNA nucleotides;
(b) a 5′-wing region (A′) comprising 2 to 6 locked nucleotides or 2′ substituted nucleosides; and
(c) a 3′-wing region (C′) comprising 2 to 6 locked nucleotides or 2′ substituted nucleosides;
wherein the central region of the ASO is at least 80% complementary or hybridizes to a target RNA sequence; and wherein the ASO comprises at least one modified nucleotide selected from:

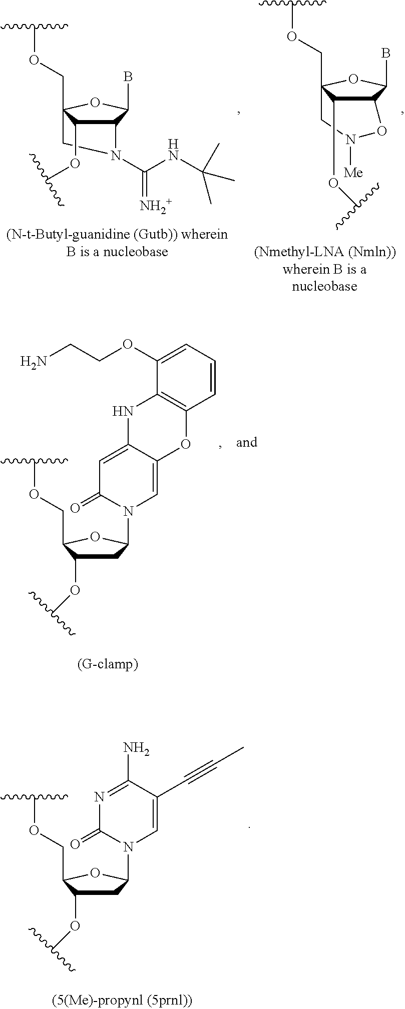

2. The ASO of claim 1, wherein (i) the central region (B′) comprises a modified nucleotide selected from G-clamp and 5prnl, (ii) the 5′-wing region (A′) comprises a modified nucleotide selected from Gutb and Nmln, (iii) the 3′-wing region (C′) comprises a modified nucleotide selected from Gutb and Nmln, or (iv) any combination thereof.

3. The ASO of claim 1, wherein the central region (B′) comprises 2, 3, 4, 5, or 6 or more modified nucleotides.

4. The ASO of claim 1, wherein the 5′-wing region (A′), the 3′-wing region (C′), or both comprise a modified nucleotide selected from Gutb, Nmln, G-clamp, and 5prnl.

5. The ASO of claim 1, wherein the ASO molecule further comprises 1 or more phosphorothioate (ps) internucleoside linkages, mesyl phosphoroamidate (yp) internucleoside linkages, or a combination thereof.

6. (canceled)

7. An antisense oligonucleotide (ASO) comprising 14-22 nucleotide units, wherein the ASO comprises:
(a) a central region (B′) comprising 6 or more contiguous DNA nucleotides, wherein at least one of the contiguous DNA nucleotides is a modified nucleotide,
(b) a 5′-wing region (A′) comprising 2 to 6 locked nucleotides or 2′ substituted nucleosides, and
(c) a 3′-wing region (C′) comprising 2 to 6 locked nucleotides or 2′ substituted nucleosides,
wherein the central region of the ASO is at least 80% complementary or hybridizes to a target RNA sequence, and wherein the ASO comprises 1, 2, 3, 4, 5, 6, 7, 8, 9, 10, 11, 12, 13, 14, 15, or more mesyl phosphoroamidate (yp) internucleoside linkages.

8. The ASO of claim 7, wherein the ASO comprises at least 1, at least 2, at least 3, at least 4, or at least 5 or more nucleotide(s) selected from:

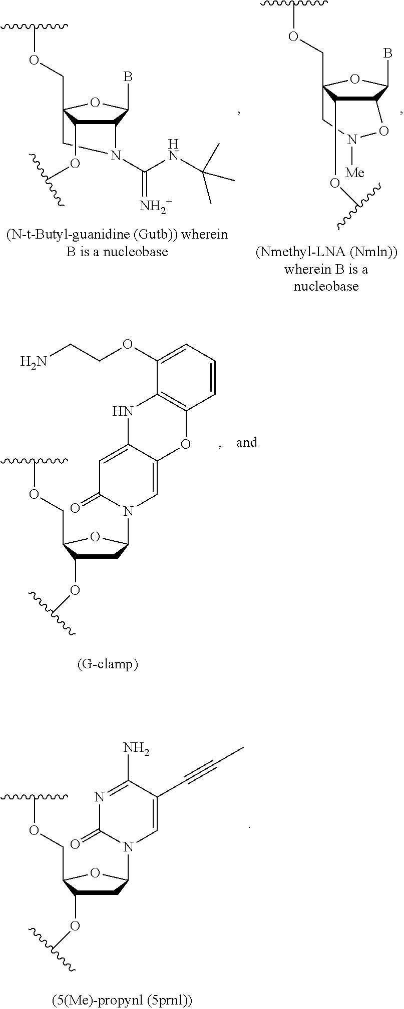

9. The ASO of claim 7, wherein the ASO molecule further comprises 1 or more phosphorothioate (ps) internucleoside linkages

10. The ASO of claim 1, wherein:
(i) at least one mesyl phosphoroamidate (yp) internucleotide linkage is between nucleoside positions 3 and 4 from the 5′ end of the ASO molecule;
(ii) at least one mesyl phosphoroamidate (yp) internucleotide linkage is between nucleoside positions 5 and 6 from the 5′ end of the ASO molecule;
(iii) at least one mesyl phosphoroamidate (yp) internucleotide linkage is between nucleoside positions 6 and 7 from the 5′ end of the ASO molecule;
(iv) at least one mesyl phosphoroamidate (yp) internucleotide linkage is between nucleoside positions 7 and 8 from the 5′ end of the ASO molecule;
(v) at least one mesyl phosphoroamidate (yp) internucleotide linkage is between nucleoside positions 8 and 9 from the 5′ end of the ASO molecule;
(vi) at least one mesyl phosphoroamidate (yp) internucleotide linkage is between nucleoside positions 9 and 10 from the 5′ end of the ASO molecule; or
(vii) a combination thereof.

11. The ASO of claim 1, wherein the 5′-wing region (A′), the 3′-wing region (C′), or both comprise at least one mesyl phosphoroamidate (yp) internucleotide linkage.

12. The ASO of claim 1, wherein the ASO molecule further comprises a galactosamine.

13. The ASO of claim 12, wherein the galactosamine is N-acetylgalactosamine (GalNAc) of Formula (VI): 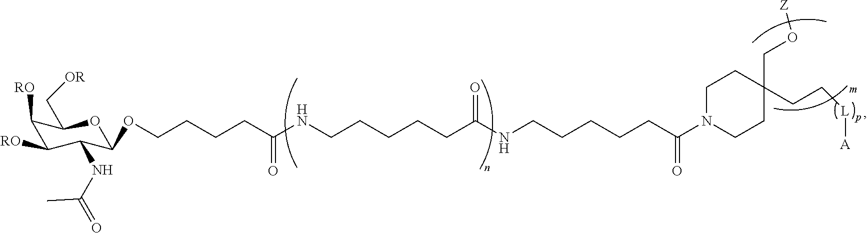
wherein
m is 1, 2, 3, 4, or 5;
each n is independently 1 or 2;
p is 0 or 1;
each R is independently H;
each Y is independently selected from —O—P(═O)(SH)—, —O—P(═O)(O)—, —O—P(═O)(OH)—, and —O—P(S)S—;
Z is H or a second protecting group;
either L is a linker or L and Y in combination are a linker; and
A is H, OH, a third protecting group, an activated group, or an oligonucleotide; or wherein the galactosamine is N-acetylgalactosamine (GalNAc) of Formula VII: 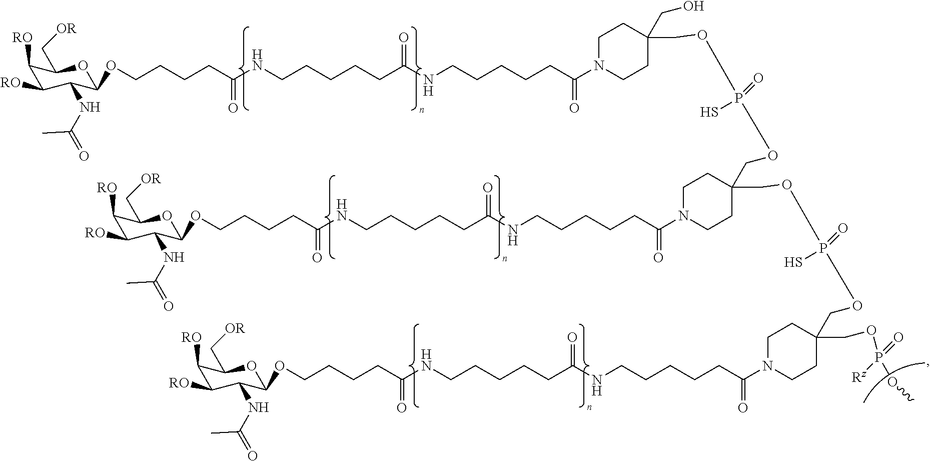
wherein R^{z} is OH or SH; and each n is independently 1 or 2.

14. (canceled)

15. The ASO of claim 1, wherein;
(i) the target RNA sequence is a viral gene;
(ii) the target RNA sequence is a gene is from a DNA virus;
(iii) the target RNA sequence is a gene from a double-stranded DNA (dsDNA) virus;
(iv) the target RNA sequence is a gene from a hepadnavirus;
(v) the target RNA sequence is a gene from a hepatitis B virus (HBV);
(vi) the target RNA sequence is a gene from a HBV of any one of genotypes A-J; or
(vii) the target RNA sequence is selected from the S gene or X gene of a HBV.

16. The ASO of claim 1, wherein the target RNA sequence is selected from a gene encoding a Methylation-Controlled J protein (MCJ protein), a gene encoding TAZ, a gene encoding angiopoietin like 3 (ANGPTL3), a gene encoding diacylglycerol acyltransferase 2 (DGAT2), and a gene encoding hydroxysteroid 17-beta dehydrogenase 13 (HSD17B13).

17. The ASO of claim 1, wherein (i) the 5′-wing region of the ASO comprises 2 to 6 phosphorothioate-linked locked nucleosides, (ii) the 3′-wing region of the ASO comprises 2 to 6 phosphorothioate-linked locked nucleosides, or (iii) a combination thereof, wherein the locked nucleosides are selected from LNA, ScpBNA, AmNA, AmNA (N-Me), GuNA, GuNA (N—R¹¹) where R¹¹ is selected from Me, Et, i-PR, t-Bu and combinations thereof.

18. (canceled)

19. The ASO of claim 1, wherein the central region of the ASO comprises at least 5 contiguous phosphorothioate-linked DNA nucleotides, at least 5 contiguous mesyl phosphoroamidate-linked DNA nucleotides, or at least 5 contiguous DNA nucleotides linked by one or more phosphorothioate internucleoside linkages and one or more mesyl phosphoroamidate internucleoside linkages.

20. The ASO of claim 7, wherein the central region of the ASO comprises 8 to 10 contiguous phosphorothioate-linked DNA nucleotides, 8 to 10 contiguous mesyl phosphoroamidate-linked DNA nucleotides, or 8 to 10 DNA nucleotides linked by one or more phosphorothioate internucleoside linkages and one or more mesyl phosphoroamidate internucleoside linkages.

21-23. (canceled)

24. A pharmaceutical composition comprising the ASO of claim 1; and a pharmaceutically acceptable excipient.

25-27. (canceled)

28. A method of treating a subject having a Hepatitis B virus (HBV) infection, comprising administering to the subject with HBV an ASO according to claim 1.

29-33. (canceled)

34. A method of decreasing expression of a target gene in a subject, comprising administering to the subject an ASO according to claim 1.

35-43. (canceled)
